(19) United States
(12) Patent Application Publication
(10) Pub. No.: US 2025/0376435 A1
(43) Pub. Date: Dec. 11, 2025

(54) ORGANIC COMPOUNDS AND ORGANIC LIGHT EMITTING DIODE COMPRISING THE SAME

(71) Applicant: MATERIAL SCIENCE CO., LTD., Gyeonggi-do (KR)

(72) Inventors: Seung Hyun KIM, Gyeonggi-do (KR); Ki Hoon SHIN, Gyeonggi-do (KR); Gi Rim LEE, Gyeonggi-do (KR); Hee Jin YANG, Gyeonggi-do (KR); Hee Jun PARK, Gyeonggi-do (KR); Woo Hyoung CHO, Gyeonggi-do (KR); Jie SONG, Gyeonggi-do (KR)

(21) Appl. No.: 19/229,123

(22) Filed: Jun. 5, 2025

(30) Foreign Application Priority Data
Jun. 7, 2024 (KR) 10-2024-0074293
Dec. 31, 2024 (KR) 10-2024-0202651

(51) Int. Cl. C07C 211/54; C07C 211/45; C07C 211/55; C07C 211/56; C07C 211/60; H10K 50/858

(52) U.S. Cl. CPC C07C 211/54; C07C 211/45; C07C 211/55; C07C 211/56; C07C 211/60; C07C 2601/14; C07C 2602/10; C07C 2603/74; H10K 50/858

(57) ABSTRACT

The present disclosure is to provide a novel organic compound and an organic light-emitting diode including the same. An organic light-emitting diode according to one embodiment of the present disclosure includes a first electrode, a second electrode facing the first electrode, at least one organic material layer positioned on the inner side of the first electrode and the second electrode, and a capping layer positioned on the outer side of at least one of the first electrode and the second electrode, wherein the capping layer includes the novel organic compound.

### BACKGROUND

### 1. Field

The present disclosure relates to an organic compound and an organic light-emitting diode including the same.

### 2. Description of the Related Art

An organic light-emitting diode (OLED) has a simpler structure compared to other flat panel display devices such as a liquid crystal display (LCD), a plasma display panel (PDP), and a field emission display (FED), and has various advantages in terms of a manufacturing process, and has high luminance, excellent viewing angle characteristics, fast response speed, and a low operation voltage, and thus is being actively developed and commercialized as a light source of a backlight, lighting, and billboards, in a flat panel display such as a wall-mountable television or a display.

The organic light-emitting diode is composed of two electrodes, and an organic material layer therebetween. Electrons and holes from two electrodes are injected into a light-emitting layer in which excitons are generated via recombination of electrons and holes. When the generated excitons change from an excited state to a ground state, the light is generated.

The organic light-emitting diode may include at least one light-emitting layer. In general, the organic light-emitting diode having a plurality of light-emitting layers includes light-emitting layers that emit light beams with different peak wavelengths. Thus, a specific color may be rendered via a combination of the light beams with the different peak wavelengths.

The organic light-emitting diodes may be classified into a top emission type light-emitting diode and a bottom emission type light-emitting diode. The top emission type light-emitting diode emits light generated in the light-emitting layer toward a translucent anode using a reflective cathode. On the other hand, in the bottom emission type light-emitting diode, light generated in the light-emitting layer is reflected from a reflective anode to be directed toward a transparent cathode, that is, toward a driving thin film transistor.

With the development of display devices, the need for a capping layer compound that may improve the emission efficiency and lifetime of an organic light-emitting diode is increasing. Conventionally, high-refractive compounds were used to diffuse light from a panel, thereby increasing light transmittance, and suppressing light absorption within the diode to increase the efficiency of the diode. However, in order to increase light efficiency, the need for low-refractive compounds that may increase the efficiency of the diode by recollecting the light diffused by the high-refractive compound and transmitting to a screen is increasing.

### SUMMARY

An embodiment of the present disclosure is to provide a novel organic compound and an organic light-emitting diode including the same.

Another embodiment according to the present disclosure may be used for accomplishing other tasks particularly unmentioned in addition to the above-described task.

Purposes of the present disclosure are not limited to the above-mentioned purpose. Other purposes and advantages of the present disclosure that are not mentioned may be understood based on the following descriptions, and may be more clearly understood based on embodiments of the present disclosure. Further, it will be easily understood that the purposes and advantages of the present disclosure may be realized using means shown in the claims and combinations thereof.

A compound according to one embodiment of the present disclosure is represented by Chemical Formula 1 below.

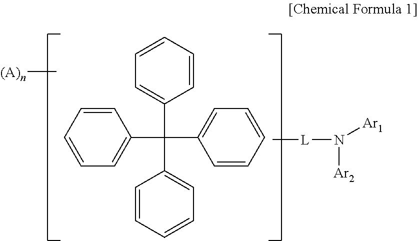

wherein in the Chemical Formula 1,
n is an integer of 1 to 20,
A is an alkyl group having 1 to 30 carbon atoms, or a cycloalkyl group having 3 to 20 carbon atoms,
L is selected from the group consisting of a single bond, a substituted or unsubstituted arylene group having 6 to 30 carbon atoms, a substituted or unsubstituted arylalkyl group having 7 to 30 carbon atoms, a substituted or unsubstituted heteroarylene group having 5 to 60 carbon atoms and a substituted or unsubstituted heteroarylalkylene group having 6 to 60 carbon atoms,
Ar₁ and Ar₂ are identical with or different from each other, and are each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms and a substituted or unsubstituted aryloxy group having 6 to 30 carbon atoms, or combined with an adjacent group from each other to form a substituted or unsubstituted ring, and

the substituents of A, Ar₁, and Ar₂ are each independently at least one selected from the group consisting of deuterium, a cyano group, a nitro group, a halogen group, a hydroxyl group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an arylalkyl group having 6 to 30 carbon atoms, a cycloalkyl group having 3 to 20 carbon atoms, a heterocycloalkyl group having 3 to 20 carbon atoms, an aryl group having 6 to 30 carbon atoms, a heteroaryl group having 2 to 30 carbon atoms, a heteroarylalkyl group having 3 to 30 carbon atoms, an alkoxy group having 1 to 30 carbon atoms, an alkylsilyl group having 1 to 30 carbon atoms, an arylsilyl group having 6 to 30 carbon atoms, and an aryloxy group having 6 to 30 carbon atoms, where when a plurality of substituents are introduced, the substituents are identical with or different from each other and combined with an adjacent group from each other to form a substituted or unsubstituted ring.

An organic light-emitting diode including an organic compound according to one embodiment of the present disclosure may have excellent driving voltage, emission efficiency, external quantum efficiency (EQE) and stability, and may have long lifetime characteristics.

In addition, the organic compound according to one embodiment of the present disclosure may exhibit low refractive index characteristics in which a refractive index (n) is 1.50 or more and 1.80 or less at a wavelength of 400 nm to 650 nm, and high transmittance characteristics in which a light transmittance is about 80% or more at a wavelength of 400 nm to 650 nm.

The effect of the present description is not limited to the effects mentioned above, and other effects not mentioned will be clearly understood by those skilled in the art from the description below.

### BRIEF DESCRIPTION OF DRAWINGS

FIG. 1 is a cross-section of a thin film formed by Compound 1, photographed using a scanning electron microscope.

FIG. 2 is a photographic image of a thin film formed by Compound 2 during a deposition process.

### DETAILED DESCRIPTION

The above-mentioned purposes, features and advantages are described in detail below, and accordingly, those skilled in the art in the technical field to which the present disclosure belongs will be able to easily implement the technical ideas of the present disclosure.

Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, “including”, “contain”, “containing”, etc. when used in this specification, specify the presence of the stated features, integers, operations, elements, and/or components, but do not preclude the presence or addition of one or more other features, integers, operations, elements, components, and/or portions thereof.

In descriptions of temporal relationships, for example, temporal precedent relationships between two events such as “after”, “subsequent to”, “before”, etc., another event may occur therebetween unless “directly after”, “directly subsequent” or “directly before” is not indicated.

In interpreting a numerical value, the value is interpreted as including an error range unless there is no separate explicit description thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the description, when an element is referred to as being “on (or under)” or “above (or below)” another arbitrary element, the arbitrary element can be disposed to contact with the top (or bottom) of the element, or intervening elements may also be present between the element and the arbitrary element disposed on (or under) the element.

As used herein, the term “halogen group” refers to fluorine, chlorine, bromine and iodine.

As used herein, the term “alkyl group” refers to both a linear alkyl radical and a branched alkyl radical. Unless otherwise specifically limited, the alkyl group contains 1 to 30 carbon atoms, and may include methyl, ethyl, propyl, isopropyl, butyl, sec-butyl, isobutyl, tert-butyl, pentyl, isoamyl, hexyl, etc., without limitation. In addition, the alkyl group may be optionally substituted.

As used herein, the term “cycloalkyl group” refers to a cyclic alkyl radical. Unless otherwise specifically limited, the cycloalkyl group contains 3 to 20 carbon atoms and may include cyclopropyl, cyclobutyl, cyclopentyl, cyclohexyl, norbornyl, adamantyl, etc, without limitation. In addition, the cycloalkyl group may be optionally substituted.

As used herein, the term “alkenyl group” refers to both a linear alkene radical and a branched alkene radical, having at least one carbon-carbon double bond. Unless otherwise specifically limited, the alkenyl group contains 2 to 30 carbon atoms and may include vinyl, allyl, isopropenyl, 2-butenyl, etc., without limitation. In addition, the alkenyl group may be optionally substituted.

As used herein, the term “cycloalkenyl group” refers to a cyclic alkenyl radical. Unless otherwise specifically limited, the cycloalkenyl group contains 3 to 20 carbon atoms. In addition, the cycloalkenyl group may be optionally substituted.

As used herein, the term “alkynyl group” refers to both a linear alkyne radical and a branched alkyne radical, having at least one carbon-carbon triple bond. Unless otherwise specifically limited, the alkynyl group contains 2 to 30 carbon atoms and may include ethynyl, 2-propynyl, etc., without limitation. In addition, the alkynyl group may be optionally substituted.

As used herein, the term “cycloalkynyl group” refers to a cyclic alkynyl radical. Unless otherwise specifically limited, the cycloalkynyl group contains 3 to 20 carbon atoms. In addition, the cycloalkynyl group may be optionally substituted.

As used herein, the terms “aralkyl group” and “arylalkyl group” are inter-mixed and refer to an alkyl group having an aromatic group as a substituent. In addition, the aralkyl group (arylalkyl group) may be optionally substituted.

As used herein, the term “aryl group” or “aromatic group” is used to have the same meaning, and the aryl group includes both a monocyclic group and a polycyclic group. The polycyclic group may include a “fused ring” of two or more rings, in which two carbon atoms are common in two adjacent rings. In addition, a simple pendant type or a fused type of two or more rings may be included. Unless otherwise specifically limited, the aryl group contains 6 to 30 carbon atoms and may include phenyl, naphthyl, anthracenyl, phenanthrenyl, fluorenyl, dimethylfluorenyl, diphenylfluorenyl, spirofluorenyl, etc., without limitation. In addition, the aryl group may be optionally substituted.

As used herein, the term “heteroaryl group” or “heteroaromatic group” is used to have the same meaning, and the heteroaryl group includes both a monocyclic group and a polycyclic group. The polycyclic group may include a “fused ring” of two or more rings, in which two carbon atoms or heteroatoms are common in two adjacent rings. In addition, a simple pendant type or a fused type of two or more rings may be included. Unless otherwise specifically defined, a heteroaryl group contains 1 to 30 carbon atoms, and if the carbon atoms are 1 or 2, additional heteroatoms may be included to form rings. In addition, the heteroaryl group may contain 1 to 30 carbon atoms, wherein at least one carbon in the ring is substituted with a heteroatom such as oxygen (O), nitrogen (N), sulfur(S), or selenium (Se), and may be a 6-membered monocyclic ring such as pyridinyl, pyrazinyl, pyrimidinyl, pyridazinyl, and triazinyl, a polycyclic ring such as phenoxathinyl, indolizinyl, indolyl, purinyl, quinolyl, isoquinolyl, benzoxyzolyl, benzothiazolyl, dibenzoxyzolyl, dibenzothiazolyl, benzoimidazolyl, benzofuranyl, dibenzofuranyl, benzothiophenyl, dibenzothiophenyl, phenylcarbazolyl, 9-phenylcarbazolyl, and carbazolyl, and 2-furanyl, N-imidazolyl, 2-isoxazolyl, 2-pyridinyl, 2-pyrimidinyl, etc., without limitation. In addition, the heteroaryl group may be optionally substituted.

As used herein, the term “heterocyclic group” means that at least one of carbon atoms constituting an aryl group, a cycloalkyl group, a cycloalkenyl group, a cycloalkynyl group, an arylalkyl group, an arylamino group, etc. is substituted with a heteroatom including oxygen (O), nitrogen (N), sulfur(S), selenium (Se), etc., and may include, referring to the above-described definition, a heteroaryl group, a heterocycloalkyl group, a heterocycloalkenyl group, a heterocycloalkynyl group, a heteroarylalkyl group, a heteroarylamino group, etc., without limitation. In addition, the heterocyclic group may be optionally substituted.

As used herein, the term “carbon ring” may be used as a term including both a “cycloalkyl group,” which is an aliphatic cyclic group, and an “aryl group (aromatic group),” which is an aromatic cyclic group, unless otherwise limited.

As used herein, the terms “heteroalkyl group,” and “heteroarylalkyl group” mean that at least one constituent carbon atom is substituted with a heteroatom including oxygen (O), nitrogen (N), sulfur(S), selenium (Se) etc. In addition, the heteroalkyl group, and the heteroaralkyl group may be optionally substituted.

As used herein, the terms “alkylamino group,” “arylalkylamino group,” “arylamino group,” and “heteroarylamino group” refer to an amino group (or amine group) in which an alkyl group, arylalkyl group, aryl group, or heteroaryl group is substituted, and include all primary, secondary and tertiary amino groups (or amine groups). In addition, an alkylamino group, arylalkylamino group, arylamino group, and heteroarylamino group may be optionally substituted.

The terms used in the description of “alkylsilyl group,” “arylsilyl group,” “alkoxy group,” “aryloxy group,” “alkylthio group” and “arylthio group” mean a silyl group, an oxy group and a thio group, in which an alkyl group or aryl group is substituted. In addition, an alkylsilyl group, arylsilyl group, alkoxy group, aryloxy group, alkylthio group and arylthio group may be optionally substituted.

The terms used in the description of “arylene group,” “arylalkylene group,” “heteroarylene group,” and “heteroarylalkylene group” mean divalent substituents, in which each of the aryl group, arylalkyl group, heteroaryl group and heteroarylalkyl group further includes one more substituent. In addition, the arylene group, arylalkylene group, heteroarylene group and heteroarylalkylene group may be optionally substituted.

As used herein, the term “substituted” means that a hydrogen (H) atom bonded to a carbon or nitrogen atom of the compound of the present disclosure is substituted with a substituent other than hydrogen, and if a plurality of substituents are present, each substituent may be all identical with or different from each other.

The substituents are each independently substituted with at least one substituent selected from the group consisting of deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, and if substituted with a plurality of substituents, the substituents may be identical with or different from each other, and may combine with an adjacent group to form a substituted or unsubstituted ring.

Each subject and substituent defined in this description may be identical with or different unless otherwise specified.

In this description, unless otherwise specified, the standard for a unit is based on weight (wt). For example, if described as “%,” it is interpreted as weight % (wt %).

Hereinafter, an organic compound and an organic light-emitting diode including the same according to the present disclosure will be explained in detail.

The organic compound according to one embodiment of the present disclosure may be represented by Chemical Formula 1 below.

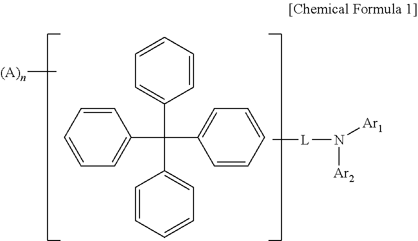

In Chemical Formula 1 above, n is an integer of 1 to 20,
A is an alkyl group having 1 to 30 carbon atoms, or a cycloalkyl group having 3 to 20 carbon atoms,
L is selected from the group consisting of a single bond, a substituted or unsubstituted arylene group having 6 to 30 carbon atoms, a substituted or unsubstituted arylalkyl group having 7 to 30 carbon atoms, a substituted or unsubstituted heteroarylene group having 5 to 60 carbon atoms and a substituted or unsubstituted heteroarylalkylene group having 6 to 60 carbon atoms,
Ar₁ and Ar₂ are identical with or different from each other, and are each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms and a substituted or unsubstituted aryloxy group having 6 to 30 carbon atoms, or combined with an adjacent group from each other to form a substituted or unsubstituted ring, and
the substituents of A, Ar₁, and Ar₂ are each independently at least one selected from the group consisting of deuterium, a cyano group, a nitro group, a halogen group, a hydroxyl group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an arylalkyl group having 6 to 30 carbon atoms, a cycloalkyl group having 3 to 20 carbon atoms, a heterocycloalkyl group having 3 to 20 carbon atoms, an aryl group having 6 to 30 carbon atoms, a heteroaryl group having 2 to 30 carbon atoms, a heteroarylalkyl group having 3 to 30 carbon atoms, an alkoxy group having 1 to 30 carbon atoms, an alkylsilyl group having 1 to 30 carbon atoms, an arylsilyl group having 6 to 30 carbon atoms, and an aryloxy group having 6 to 30 carbon atoms, where if a plurality of substituents are introduced, the substituents are identical with or different from each other and combined with an adjacent group from each other to form a substituted or unsubstituted ring.

The organic light-emitting diode according to one embodiment of the present disclosure includes a first electrode, a second electrode facing the first electrode, at least one organic material layer positioned on the inner side of the first electrode and the second electrode, and a capping layer positioned on the outer side of at least one of the first electrode and the second electrode. The capping layer includes the compound represented by Chemical Formula 1. In the light-emitting diode, detailed description on each electrode and layer will be given later.

In Chemical Formula 1, n may be, for example, an integer of 1 to 8, 1 to 6, 1 to 4, 1 to 3, 2 to 8, 2 to 6, 2 to 4, or 2 to 3.

In Chemical Formula 1, L may be, for example, a single bond (direct bond), a substituted or unsubstituted phenylene group, a substituted or unsubstituted biphenylene group, or a substituted or unsubstituted triphenylene group. Here, L being a single bond (direct bond) means that the elements of the chemical formula on both sides based on L are directly bonded, as in the case where L is absent in the chemical formula. This may be confirmed by Chemical Formula 1-1 below, etc.

The substituted or unsubstituted phenylene group may be a divalent phenylene group in which two positions are substituted in a six-position phenylene group capable of making substitution bonds. The divalent phenylene group may be any one of 1,2 (ortho) substitution, 1,3 (meta) substitution, and 1,4 (para) substitution, and may be selected from the structures B1 to B3 below (in the partial compounds of B1 to B3 below, * means a part where the partial compound is combined by a single bond).

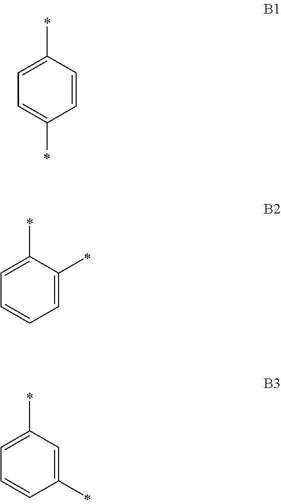

In Chemical Formula 1, A may be, for example, an alkyl group having 1 to 15, 1 to 10, or 1 to 6 carbon atoms, or a cycloalkyl group having 3 to 15, 3 to 10, or 3 to 6 carbon atoms.

Here, the alkyl group or cycloalkyl group may be, for example, a methyl group, an ethyl group, a propyl group, an isopropyl group, a butyl group, a sec-butyl group, a tert-butyl group, an isobutyl group, a 2-ethylbutyl group, a 3,3-dimethylbutyl group, a pentyl group, an isopentyl group, a neopentyl group, a tert-pentyl group, a cyclopentyl group, a 1-methylpentyl group, a 3-methylpentyl group, a 4-methyl-2-pentyl group, a hexyl group, a cyclohexyl group, a 4-methylcyclohexyl group, norbornyl, or an adamantyl group, without limitation.

In Chemical Formula 1, Ar₁ and Ar₂ may be, for example, identical with or different from each other, and may be each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms, and a substituted or unsubstituted arylalkyl group having 7 to 30 carbon atoms.

In Chemical Formula 1, Ar₁ and Ar₂ may be, for example, each independently a substituted or unsubstituted alkyl group having 1 to 15, 1 to 10, or 1 to 6 carbon atoms, a substituted or unsubstituted cycloalkyl group having 3 to 15, 3 to 10, or 3 to 6 carbon atoms, or a substituted or unsubstituted aryl group having 6 to 30, 6 to 25, or 6 to 15 carbon atoms.

Here, the alkyl group may be a methyl group, an ethyl group, an isopropyl group, a tert-butyl group, etc., without limitation.

Here, the cycloalkyl group may be, for example, a substituted or unsubstituted cyclopentyl group, a substituted or unsubstituted cyclohexyl group, a substituted or unsubstituted norbornyl group, or a substituted or unsubstituted adamantyl group, etc., without limitation.

Here, the aryl group may be, for example, a substituted or unsubstituted phenyl group, a substituted or unsubstituted biphenyl group, or a substituted or unsubstituted terphenyl group, etc., without limitation.

Here, the arylalkyl group may be, for example, each independently an aryl group having 6 to 30, 6 to 25, or 6 to 15 carbon atoms, substituted with a cycloalkyl group having 3 to 15, 3 to 10, or 3 to 6 carbon atoms or an alkyl group having 1 to 15, 1 to 10, 1 to 6, or 1 to 4 carbon atoms.

Here, for example, at least one of Ar₁ and Ar₂ may be selected from the group consisting of an unsubstituted alkyl group having 1 to 30 carbon atoms, a substituted or unsubstituted cycloalkyl group having 3 to 20 carbon atoms, and a substituted or unsubstituted arylalkyl group having 7 to 30 carbon atoms.

Here, for example, Ar₁ and Ar₂ may not each independently include a fused aryl structure. For example, the fused aryl structure may be naphthyl, fluorenyl, anthracenyl, etc.

Ar₁ and Ar₂ may each include at least one alkyl group, cycloalkyl group, or aryl group substituted with an alkyl group or a cycloalkyl group.

If Ar₁ and Ar₂ are aryl groups substituted with an alkyl group or a cycloalkyl group, the alkyl group or cycloalkyl group substituted in the aryl group may be fused with an adjacent aryl group to form a polycyclic compound. For example, 1,1,4,4-tetramethyl-1,2,3,4-tetrahydronaphthalene, etc. may be formed, but is not limited thereto.

According to one embodiment of the present disclosure, the refractive index of the compound represented by Chemical Formula 1 for light in a wavelength band of 400 nm to 650 nm may be 1.8 or less.

For example, the refractive index of the compound represented by Chemical Formula 1 for light in a wavelength band of 400 nm to 650 nm may be 1.80 or less, 1.75 or less, 1.70 or less, or 1.65 or less.

For example, the refractive index of the compound represented by Chemical Formula 1 for light in a wavelength band of 460 nm may be 1.70 or less, the refractive index of the compound represented by Chemical Formula 1 for light in a wavelength band of 520 nm may be 1.65 or less, and the refractive index of the compound represented by Chemical Formula 1 for light in a wavelength band of 620 nm may be 1.60 or less.

The compound according to embodiments may exhibit low refractive index characteristics. For example, the compound represented by Chemical Formula 1 may have a refractive index value of 1.50 or more and 1.80 or less, 1.50 or more and 1.70 or less, 1.50 or more and 1.65 or less, 1.51 or more and 1.80 or less, 1.51 or more and 1.70 or less, 1.51 or more and 1.65 or less, 1.52 or more and 1.80 or less, 1.52 or more and 1.70 or less, or 1.52 or more and 1.65 or less for light in a 460 nm wavelength band, a 520 nm wavelength band, a 620 nm wavelength band, or a 400 nm to 650 nm wavelength band.

In the organic light-emitting diode according to one embodiment, the capping layer may include the compound represented by Chemical Formula 1 and may exhibit low refractive index characteristics due to the low refractive index characteristics of the compound described above. For example, the refractive index of the capping layer for light in the wavelength band of 400 nm to 650 nm may be 1.80 or less.

In the case of an organic light-emitting diode including a capping layer having a refractive index of greater than 1.80, the emission efficiency may be lower than that of an organic light-emitting diode including a capping layer having a refractive index of 1.80 or less. However, the lower limit of the refractive index of the compound of the present disclosure and the capping layer including the compound is not separately determined, but may be, for example, 1.50 or more, 1.51 or more, or 1.52 or more. For example, the compound represented by Chemical Formula 1 and the capping layer including the same may have a refractive index value of 1.50 or more and 1.70 or less for light in the wavelength band of 400 nm to 650 nm.

The compound according to one embodiment includes a tetraphenyl structure (hereinafter, tetraphenyl-moiety, abbreviated as TM) containing at least one alkyl group or cycloalkyl group connected to nitrogen (N), so that the molecular structure of the compound has high steric hindrance, so that the space structure may be three-dimensional structure, and the sedimentation density between compound molecules may be lowered. In addition, the structure of the compound according to one embodiment may be advantageous in increasing the propagation speed of light in a medium, and may reduce the propagation speed ratio of light in a vacuum neutralized medium, so that a lower refractive index may be achieved. Furthermore, in the compound represented by Chemical Formula 1 of the present disclosure, Ar₁ and Ar₂ may be, for example, an alkyl group, a cycloalkyl group, or an aryl group substituted with an alkyl group or a cycloalkyl group, and in this case, steric hindrance may be given to all aryl structures connected to nitrogen to further lower the density, so that a lower refractive index may be achieved.

In the compound according to one embodiment, the three-dimensional structure and size of the molecule may be controlled by selecting the number of alkyl groups or cycloalkyl groups introduced into the compound. Through this, the packing density of a thin film formed by the compound and the crystallinity of the compound may be controlled.

However, if the packing density of the thin film is lowered, the refractive index may decrease, but due to the low crystallinity, a difference in deposition temperature with other materials may occur in the manufacturing process of the organic light-emitting diode, which may reduce the productivity of the diode, and also, since the diode has a low glass transition temperature, the thermal stability of the diode may be reduced during undergoing another process, so it is necessary to select the number of alkyl groups or cycloalkyl groups introduced into the compound within an appropriate range.

For example, in the compound according to one embodiment, the number of alkyl groups or cycloalkyl groups introduced into the compound may be 4 to 7, 4 to 8, or 4 to 9. Within this range, both low refractive index and thermal stability during the manufacturing process may be satisfied.

In addition, in the molecular structure of the compound according to one embodiment, an alkyl group or a cycloalkyl group may be introduced to all structures connected to nitrogen, and in this case, both low refractive index and thermal stability during the manufacturing process may be satisfied.

The compound according to one embodiment and a capping layer including the same may have a light transmittance of about 80% (absorption rate constant (K) value of 0.02) or more in a visible light region of a wavelength of 400 nm to 460 nm, thereby reducing the loss of light generated from the diode and improving the emission efficiency and external quantum efficiency of the organic light-emitting diode. The compound according to one embodiment and the capping layer including the same may have a light transmittance of 82% or more, 84% or more, 86% or more, 88% or more, 89% or more, 90% or more, 91% or more, 92% or more, 93% or more, 94% or more, 95% or more, or 96% or more in a visible light region of a wavelength of 400 nm to 460 nm.

For example, the compound according to one embodiment and the capping layer including the same may have a light transmittance of 80% or more in a visible light region of a wavelength of 400 nm to 410 nm.

In the organic light-emitting diode according to one embodiment, the capping layer exhibiting low refractive index characteristics is a layer through which light passes last in the diode, and if absorption occurs in the visible light range of 400 nm to 410 nm wavelength, the diode efficiency may be reduced.

In the case of the capping layer compound according to the conventional technology, absorption may occur in the 400 nm to 410 nm wavelength band, which is a deep blue range, but in the case of the compound according to one embodiment and the capping layer including the same, since the light transmittance in the 400 nm to 410 nm wavelength band is 80% or more, the loss of light generated in the diode may be minimized, and the efficiency of the diode may be significantly improved.

The organic compound according to one embodiment may maintain a wide band gap that cannot absorb light in the visible light wavelength band, and thus, a low refractive index may be maintained.

In addition, the compound according to one embodiment (represented by Chemical Formula 1) may absorb a high-energy wavelength with a wavelength of less than about 400 nm, and thus, the capping layer including the compound represented by Chemical Formula 1 may minimize damage to organic substances inside the organic light-emitting diode.

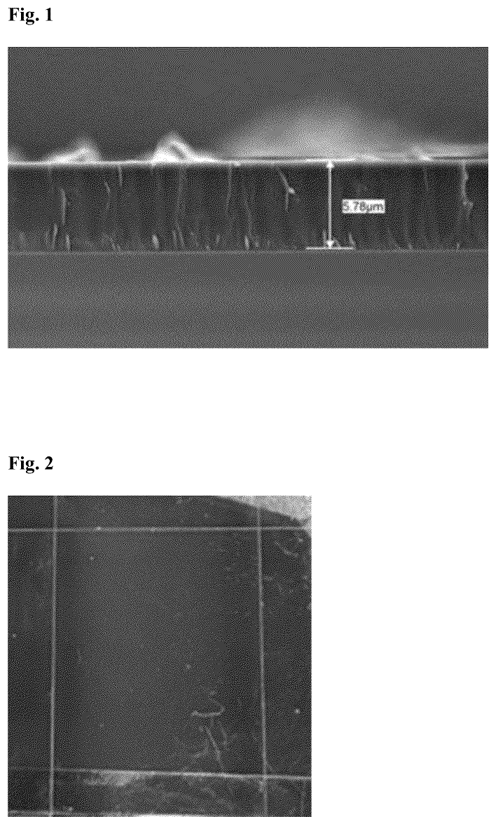

In addition, in the case of a thin film including the compound according to one embodiment, the thin film arrangement may be excellent and thus stability may be high. Through the scanning electron microscope (SEM) in FIGS. 1 and 2, a transparent and smooth cross-section of the thin film including the compound according to one embodiment may be confirmed.

In addition, the compound according to one embodiment has appropriate Tg and Td, and thus may suppress intermolecular recrystallization during the manufacturing process of the organic light-emitting diode. Therefore, the organic light-emitting diode including the capping layer according to one embodiment may have excellent color purity and greatly improve external emission efficiency.

The capping layer including the compound according to one embodiment may be positioned as a single layer or multiple layers on the surface of the first electrode or the second electrode of the organic light-emitting diode. For example, two capping layers may be positioned on one surface of the second electrode.

For example, if the organic light-emitting diode includes a plurality of capping layers, at least one of the plurality of capping layers may include at least one compound selected from the compounds represented by Chemical Formula 1. For example, in an organic light-emitting diode including a double-layer capping layer structure, the capping layer (the first capping layer) positioned on the electrode and in contact with the electrode may include the compound represented by Chemical Formula 1, and the capping layer (the second capping layer) positioned on the first capping layer may include the compound represented by Chemical Formula 1 and a material different from the compound represented by Chemical Formula 1.

Here, the material different from the compound represented by Chemical Formula 1 is not particularly limited and may be any material typically used as a capping layer compound. As non-limiting examples, the other material may be an arylamine derivative, a naphthalene derivative, an anthracene derivative, a phenanthrene derivative, a carbazole derivative, a pyridine derivative, a dibenzofuran derivative, a dibenzothiophene derivative, a pyrimidine derivative, a quinoline derivative, an isoquinoline derivative, a benzoxazole derivative, a benzothiazole derivative, a benzimidazole derivative, N4,N4′-bis[4-[bis(3-methylphenyl)amino]phenyl]-N4,N4′-diphenyl-[1,1′-biphenyl]-4,4′-diamine (DNTPD), tris(8-hydroxyquinolinato)aluminum (Alq₃), LiF, Liq, Li₂O, BaO, NaCl, or CsF.

In a structure in which the capping layer according to one embodiment includes a plurality of capping layers, the refractive indices of the capping layers may be different, and for example, the emission efficiency of the organic light-emitting diode may be further improved by utilizing the difference in refractive indices between the first capping layer material and the second capping layer material.

According to one embodiment of the present disclosure, the organic compound represented by Chemical Formula 1 may be selected from the group consisting of the compounds represented by Chemical Formulas 1-1 to 1-6 below.

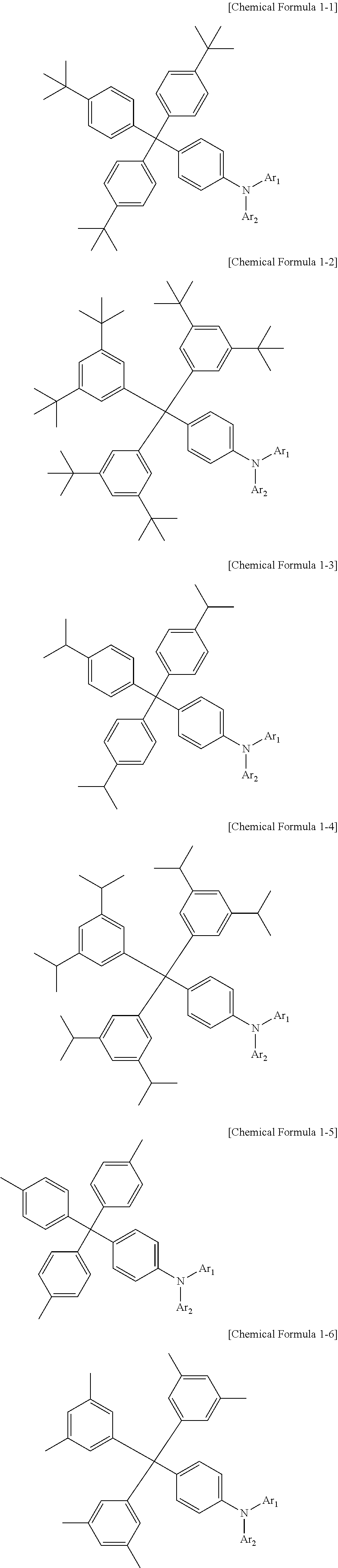

Here, for example, Ar₁ and Ar₂ are identical with or different from each other, and may be each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms, and a substituted or unsubstituted arylalkyl group having 7 to 30 carbon atoms.

Here, for example, at least one of Ar₁ and Ar₂ may be selected from the group consisting of an unsubstituted alkyl group having 1 to 30 carbon atoms, a substituted or unsubstituted cycloalkyl group having 3 to 20 carbon atoms, and a substituted or unsubstituted arylalkyl group having 7 to 30 carbon atoms.

Here, for example, Ar₁ and Ar₂ may not include a fused aryl structure.

According to one embodiment of the present disclosure, the organic compound represented by Chemical Formula 1 may be selected from the group consisting of the compounds represented by Chemical Formulas 2 to 7.

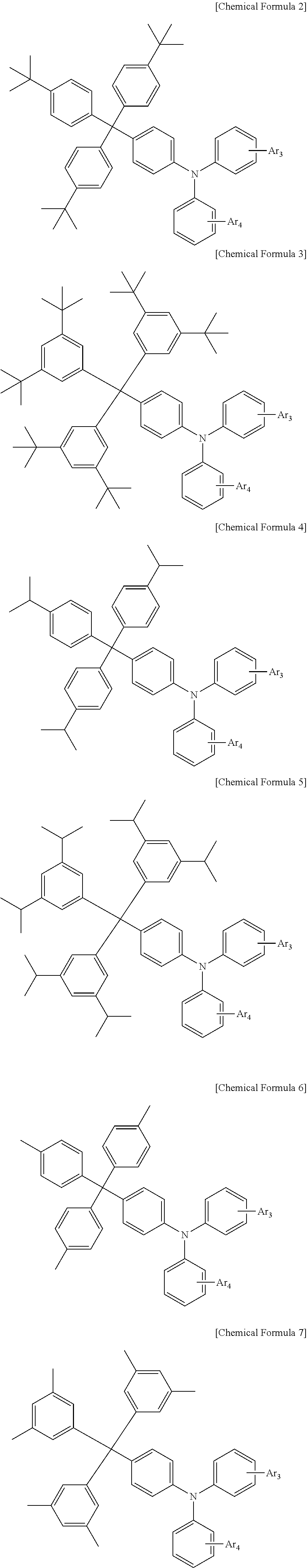

Here, Ar₃ and Ar₄ are identical with or different from each other, and are each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms, and may be combined with an adjacent group to form a substituted or unsubstituted ring. In addition, the substituents of Ar₃ and Ar₄ are the same as the substituents of Ar₁ and Ar₂ defined in Chemical Formula 1.

Here, for example, Ar₃ and Ar₄ are identical with or different from each other, and may be each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms, and a substituted or unsubstituted arylalkyl group having 7 to 30 carbon atoms.

Here, for example, at least one of Ar₃ and Ar₄ may be selected from the group consisting of an unsubstituted alkyl group having 1 to 30 carbon atoms, a substituted or unsubstituted cycloalkyl group having 3 to 20 carbon atoms, and a substituted or unsubstituted arylalkyl group having 7 to 30 carbon atoms.

Here, for example, Ar₃ and Ar₄ may not include a fused aryl structure.

According to one embodiment of the present disclosure, the tetraphenyl-moiety (hereinafter, abbreviated as TM), which is a part of the structures of Chemical Formulas 1 to 7 and Chemical Formulas 1-1 to 1-6, may be selected from the structures of TM1 to TM6 below (in the structures of TM1 to TM6 below, * indicates a part where the structure is combined with the chemical formula by a single bond).

### [Tetraphenyl-Moiety (TM)]

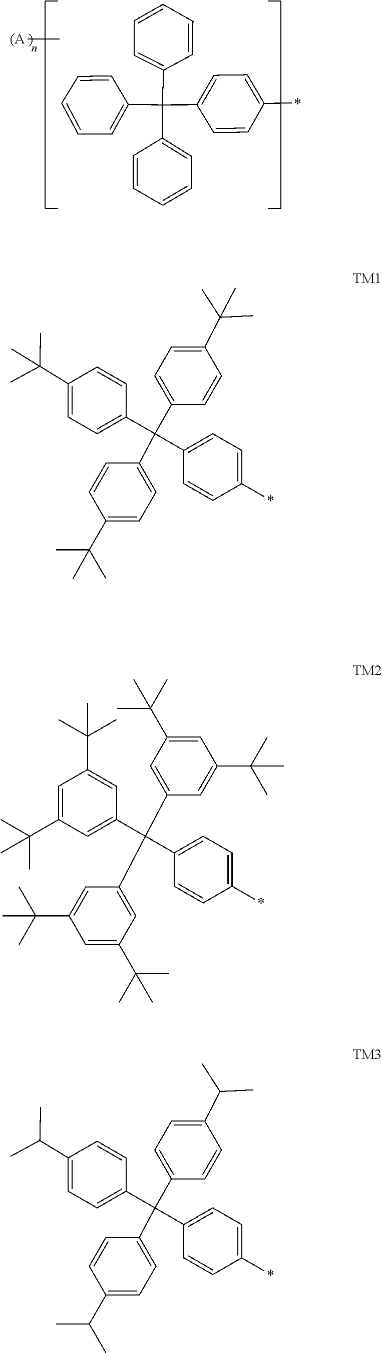

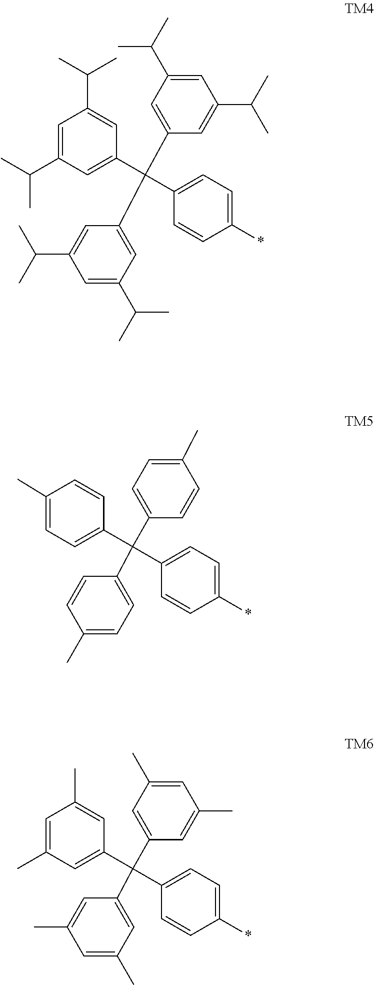

According to one embodiment of the present disclosure. Ar₁ to Ar₄ of Chemical Formulas 1 to 7 and Chemical Formulas 1-1 to 1-6 may be selected from the structures of F1 to F57 below (in the structures of F1 to F57. * indicates a part where the structure is combined with the chemical formula by a single bond).

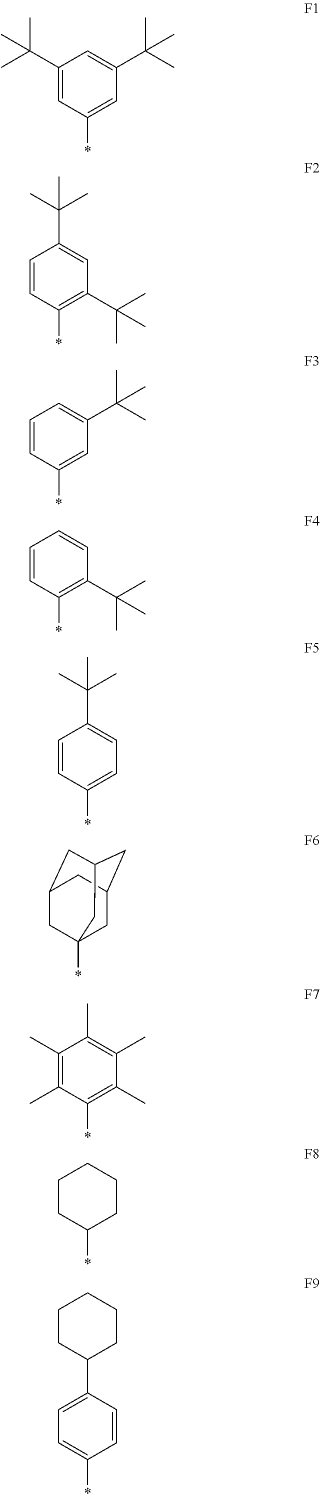

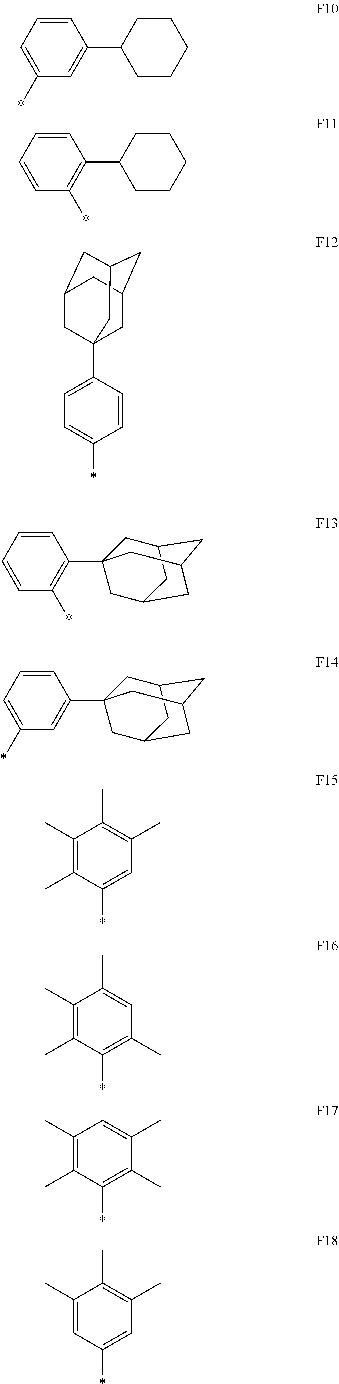

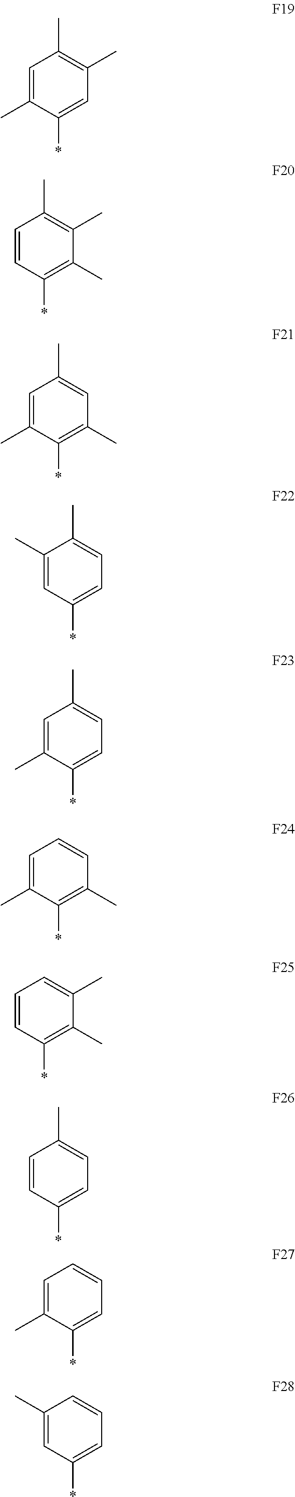

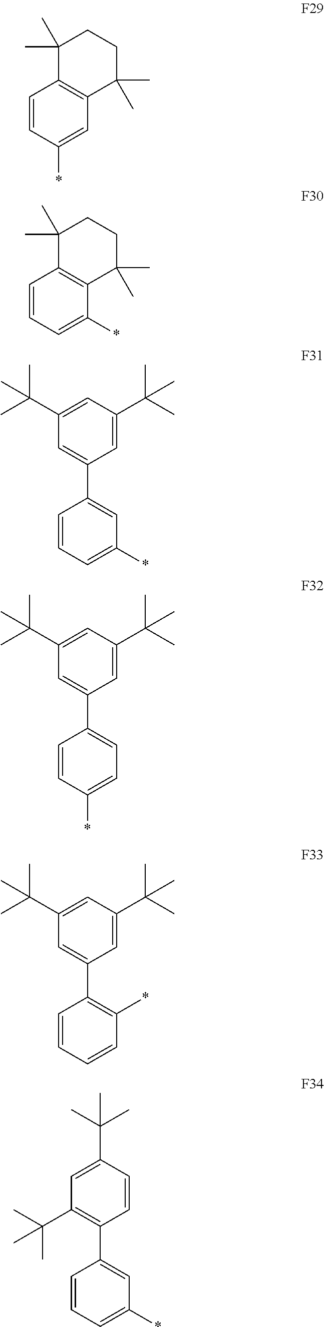

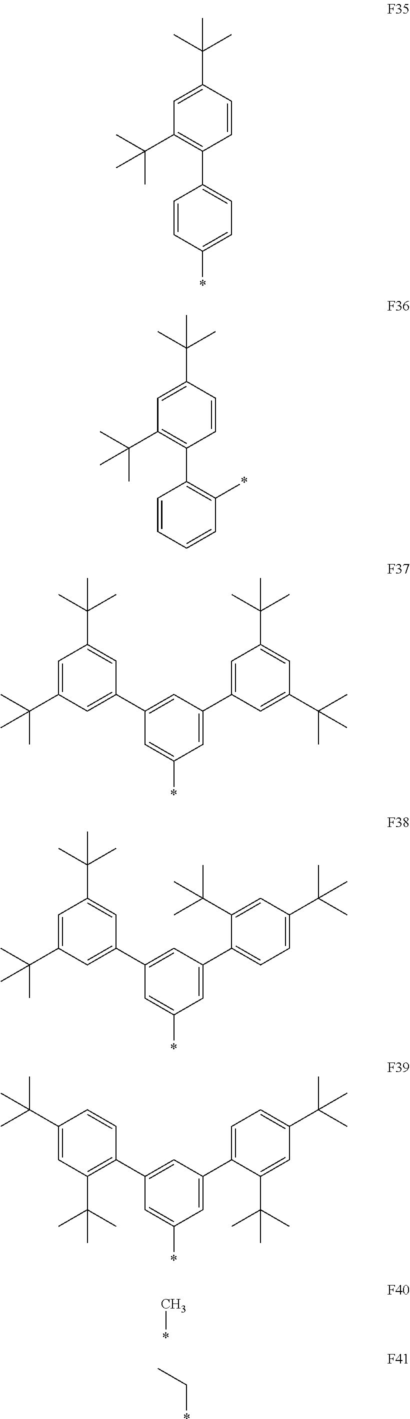

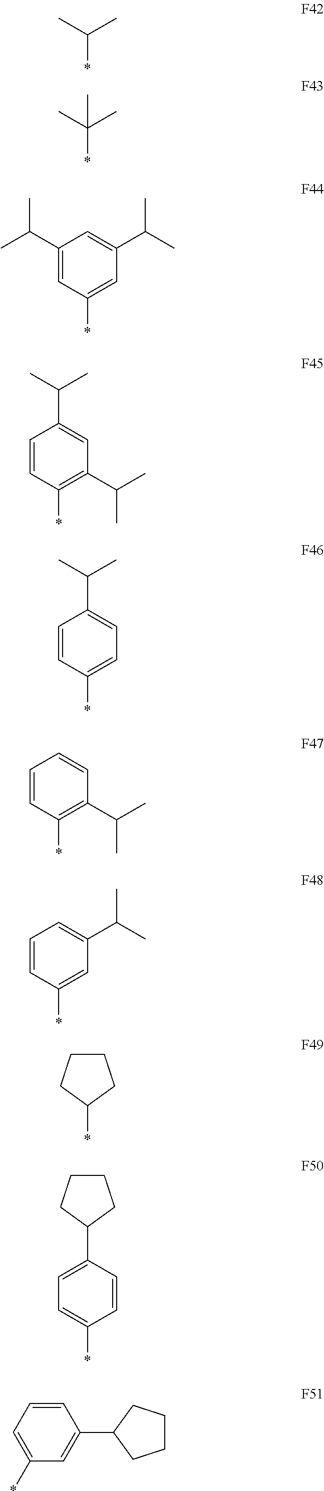

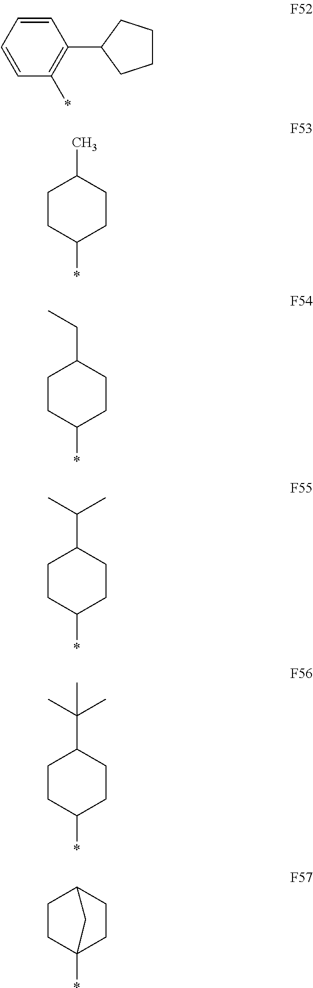

According to one embodiment of the present disclosure, the compound represented by Chemical Formula 1 may be selected from the group consisting of the compounds represented by 1 to 20 below, but is not limited thereto.

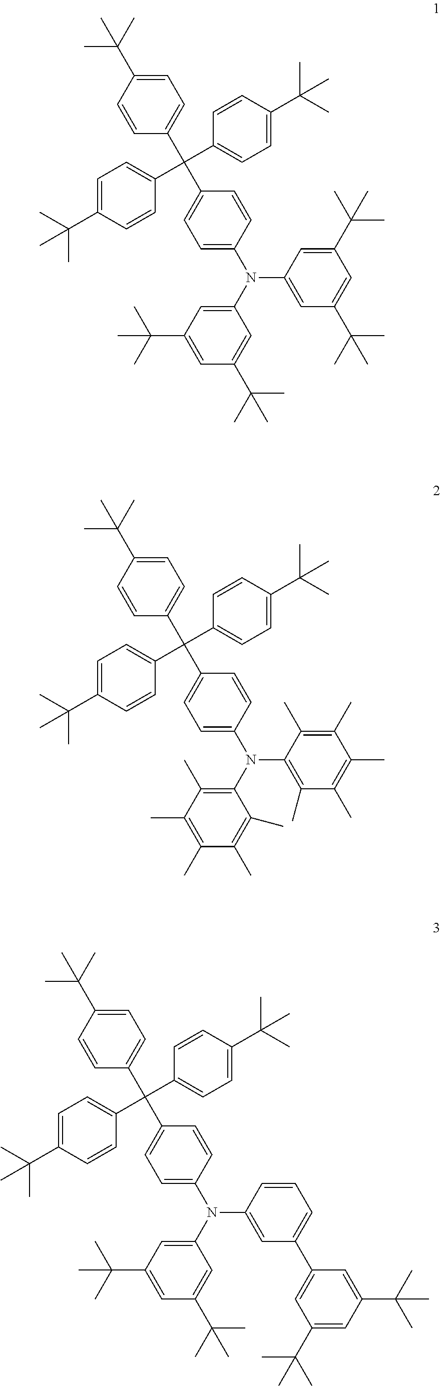

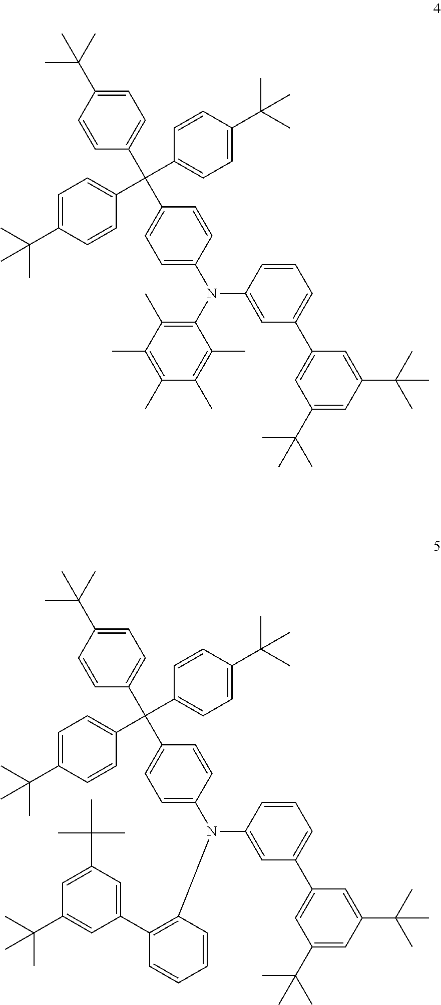

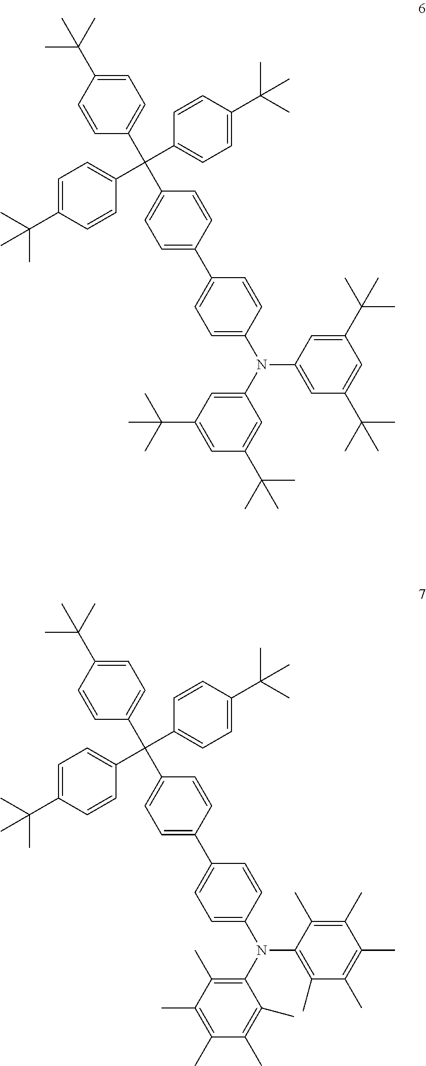

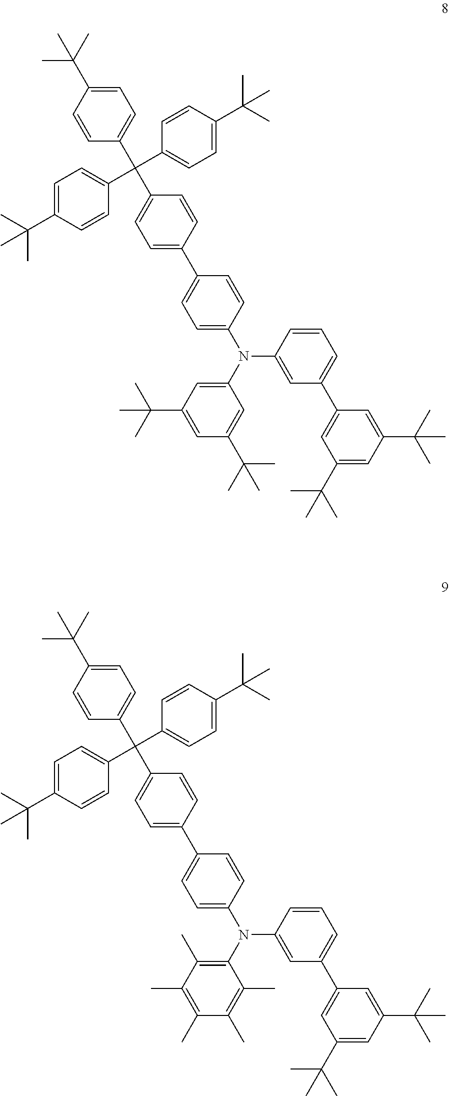

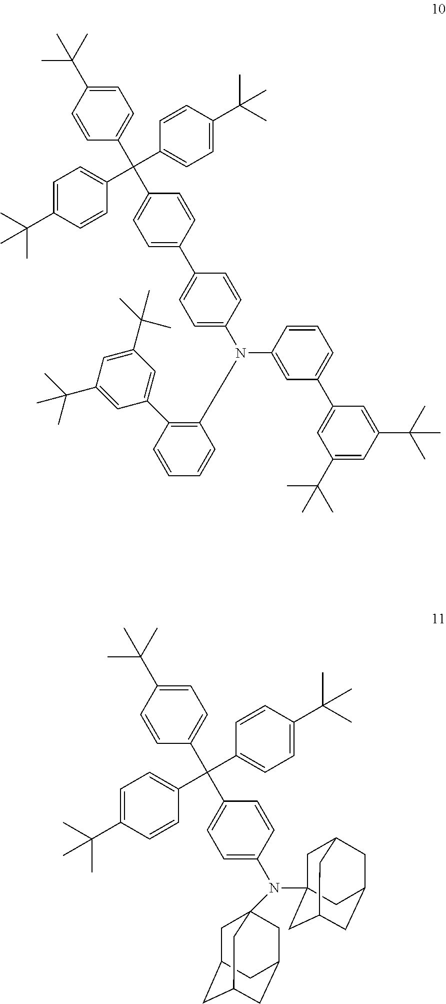

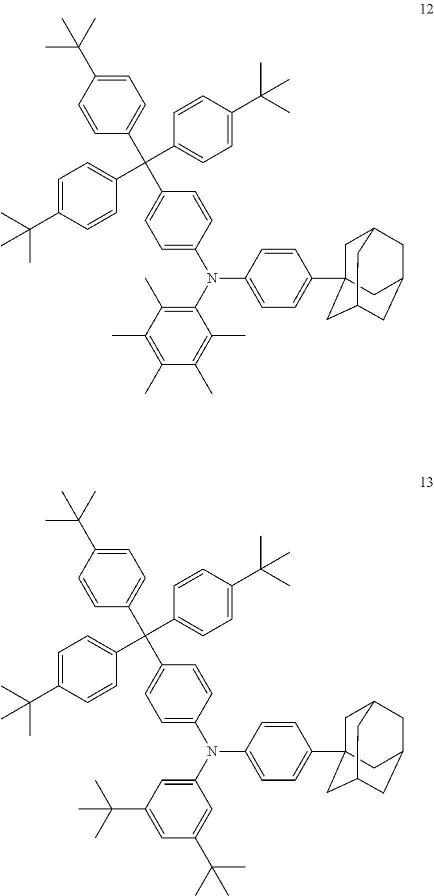 [SMILES: Cc1c(C)c(C)c(N(c2ccc(C34CC5CC(CC(C5)C3)C4)cc2)c2ccc(C(c3ccc(C(C)(C)C)cc3)(c3ccc(C(C)(C)C)cc3)c3ccc(C(C)(C)C)cc3)cc2)c(C)c1C ; CC(C)(C)c1ccc(C(c2ccc(N(c3ccc(C45CC6CC(CC(C6)C4)C5)cc3)c3cc(C(C)(C)C)cc(C(C)(C)C)c3)cc2)(c2ccc(C(C)(C)C)cc2)c2ccc(C(C)(C)C)cc2)cc1]

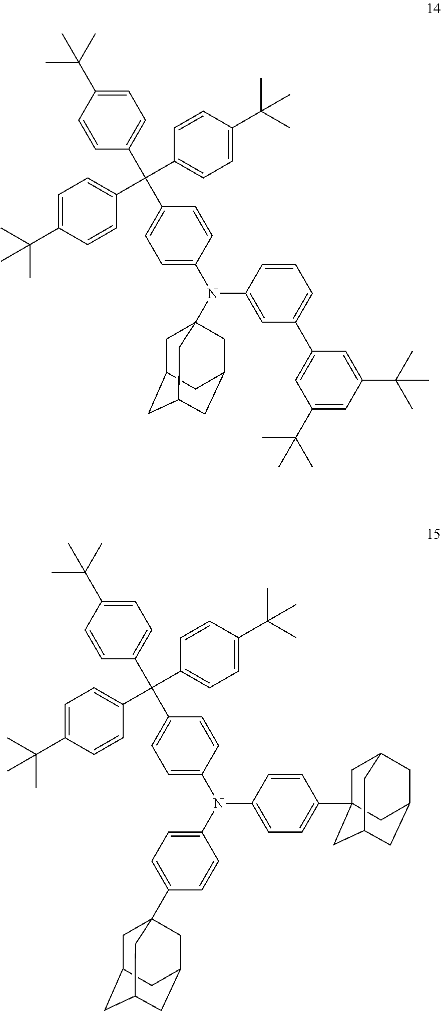

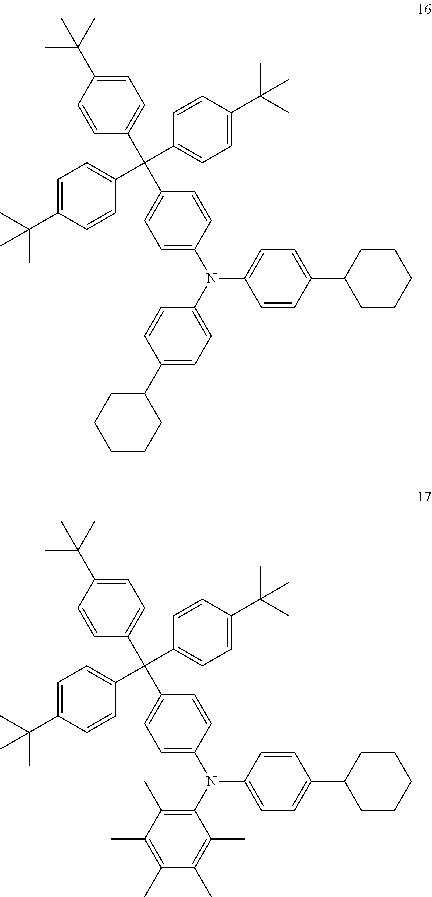 [SMILES: CC(C)(C)c1ccc(C(c2ccc(N(c3ccc(C4CCCCC4)cc3)c3ccc(C4CCCCC4)cc3)cc2)(c2ccc(C(C)(C)C)cc2)c2ccc(C(C)(C)C)cc2)cc1 ; Cc1c(C)c(C)c(N(c2ccc(C3CCCCC3)cc2)c2ccc(C(c3ccc(C(C)(C)C)cc3)(c3ccc(C(C)(C)C)cc3)c3ccc(C(C)(C)C)cc3)cc2)c(C)c1C]

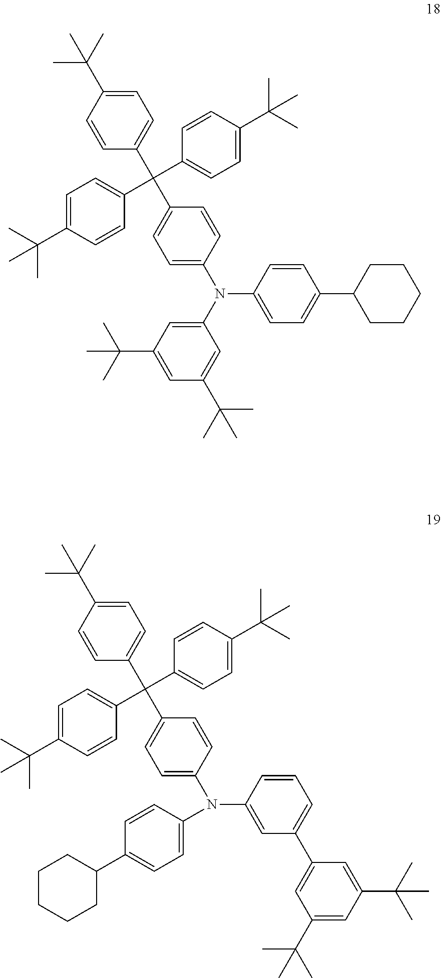 [SMILES: CC(C)(C)c1ccc(C(c2ccc(N(c3ccc(C4CCCCC4)cc3)c3cc(C(C)(C)C)cc(C(C)(C)C)c3)cc2)(c2ccc(C(C)(C)C)cc2)c2ccc(C(C)(C)C)cc2)cc1 ; CC(C)(C)c1ccc(C(c2ccc(N(c3ccc(C4CCCCC4)cc3)c3cccc(-c4cc(C(C)(C)C)cc(C(C)(C)C)c4)c3)cc2)(c2ccc(C(C)(C)C)cc2)c2ccc(C(C)(C)C)cc2)cc1]

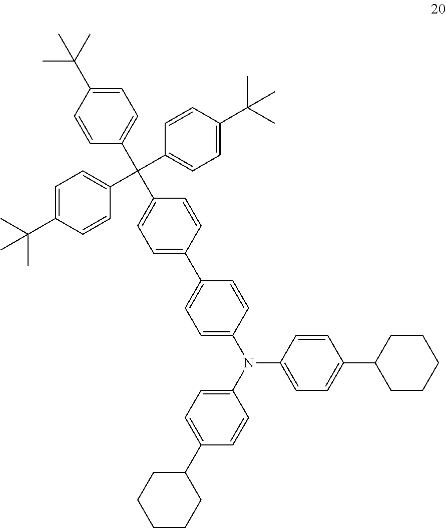 [SMILES: CC(C)(C)c1ccc(C(c2ccc(-c3ccc(N(c4ccc(C5CCCCC5)cc4)c4ccc(C5CCCCC5)cc4)cc3)cc2)(c2ccc(C(C)(C)C)cc2)c2ccc(C(C)(C)C)cc2)cc1]

The compounds of Compounds 1 to 20 may be represented as in Tables 1 and 2 below.

TABLE 1

| Compound NO. | Chemical structure | Ar₁ | Ar₂ |
|---|---|---|---|
| 1 | 1-1 | F1 | F1 |
| 2 | 1-1 | F7 | F7 |
| 3 | 1-1 | F1 | F31 |
| 4 | 1-1 | F7 | F31 |
| 5 | 1-1 | F31 | F33 |
| 11 | 1-1 | F6 | F6 |
| 12 | 1-1 | F7 | F12 |
| 13 | 1-1 | F1 | F12 |
| 14 | 1-1 | F6 | F31 |
| 15 | 1-1 | F12 | F12 |
| 16 | 1-1 | F9 | F9 |
| 17 | 1-1 | F7 | F9 |
| 18 | 1-1 | F1 | F9 |
| 19 | 1-1 | F9 | F31 |
| — | — | — | — |

TABLE 2

| Compound NO. | TM | L | Ar₁ | Ar₂ |
|---|---|---|---|---|
| 6 | TM1 | B1 | F1 | F1 |
| 7 | TM1 | B1 | F7 | F7 |
| 8 | TM1 | B1 | F1 | F31 |
| 9 | TM1 | B1 | F7 | F31 |
| 10 | TM1 | B1 | F31 | F33 |
| 20 | TM1 | B1 | F9 | F9 |

The compound represented by Chemical Formula 1 may be selected from the group consisting of the compounds represented by 21 to 1003 below, but is not limited thereto. Compounds 21 to 1003 may be represented as in Tables 3 to 13 below.

TABLE 3

| Compound NO. | Chemical structure | Ar₁ | Ar₂ |
|---|---|---|---|
| 21 | 1-1 | F1 | F2 |
| 22 | 1-1 | F1 | F3 |
| 23 | 1-1 | F1 | F4 |
| 24 | 1-1 | F1 | F5 |
| 25 | 1-1 | F1 | F6 |
| 26 | 1-1 | F1 | F7 |
| 27 | 1-1 | F1 | F10 |
| 28 | 1-1 | F1 | F11 |
| 29 | 1-1 | F1 | F13 |
| 30 | 1-1 | F1 | F14 |
| 31 | 1-1 | F1 | F29 |
| 32 | 1-1 | F1 | F32 |
| 33 | 1-1 | F1 | F33 |
| 34 | 1-1 | F1 | F34 |
| 35 | 1-1 | F1 | F35 |
| 36 | 1-1 | F1 | F36 |
| 37 | 1-1 | F1 | F37 |
| 38 | 1-1 | F1 | F38 |
| 39 | 1-1 | F1 | F39 |
| 40 | 1-1 | F1 | F40 |
| 41 | 1-1 | F1 | F42 |
| 42 | 1-1 | F2 | F2 |
| 43 | 1-1 | F2 | F3 |
| 44 | 1-1 | F2 | F4 |
| 45 | 1-1 | F2 | F5 |
| 46 | 1-1 | F2 | F6 |
| 47 | 1-1 | F2 | F7 |
| 48 | 1-1 | F2 | F9 |
| 49 | 1-1 | F2 | F10 |
| 50 | 1-1 | F2 | F11 |
| 51 | 1-1 | F2 | F12 |
| 52 | 1-1 | F2 | F13 |
| 53 | 1-1 | F2 | F14 |
| 54 | 1-1 | F2 | F29 |
| 55 | 1-1 | F2 | F31 |
| 56 | 1-1 | F2 | F32 |
| 57 | 1-1 | F2 | F33 |
| 58 | 1-1 | F2 | F34 |
| 59 | 1-1 | F2 | F35 |
| 60 | 1-1 | F2 | F36 |
| 61 | 1-1 | F2 | F37 |
| 62 | 1-1 | F2 | F38 |
| 63 | 1-1 | F2 | F39 |
| 64 | 1-1 | F2 | F40 |
| 65 | 1-1 | F2 | F42 |
| 66 | 1-1 | F3 | F3 |
| 67 | 1-1 | F3 | F4 |
| 68 | 1-1 | F3 | F5 |
| 69 | 1-1 | F3 | F6 |
| 70 | 1-1 | F3 | F7 |
| 71 | 1-1 | F3 | F9 |
| 72 | 1-1 | F3 | F10 |
| 73 | 1-1 | F3 | F11 |
| 74 | 1-1 | F3 | F12 |
| 75 | 1-1 | F3 | F13 |
| 76 | 1-1 | F3 | F14 |
| 77 | 1-1 | F3 | F29 |
| 78 | 1-1 | F3 | F31 |
| 79 | 1-1 | F3 | F32 |
| 80 | 1-1 | F3 | F33 |
| 81 | 1-1 | F3 | F34 |
| 82 | 1-1 | F3 | F35 |
| 83 | 1-1 | F3 | F36 |
| 84 | 1-1 | F3 | F37 |
| 85 | 1-1 | F3 | F38 |
| 86 | 1-1 | F3 | F39 |
| 87 | 1-1 | F3 | F40 |
| 88 | 1-1 | F3 | F42 |
| 89 | 1-1 | F4 | F4 |
| 90 | 1-1 | F4 | F5 |
| 91 | 1-1 | F4 | F6 |
| 92 | 1-1 | F4 | F7 |
| 93 | 1-1 | F4 | F9 |
| 94 | 1-1 | F4 | F10 |
| 95 | 1-1 | F4 | F11 |
| 96 | 1-1 | F4 | F12 |
| 97 | 1-1 | F4 | F13 |
| 98 | 1-1 | F4 | F14 |
| 99 | 1-1 | F4 | F29 |
| 100 | 1-1 | F4 | F31 |
| 101 | 1-1 | F4 | F32 |
| 102 | 1-1 | F4 | F33 |
| 103 | 1-1 | F4 | F34 |
| 104 | 1-1 | F4 | F35 |
| 105 | 1-1 | F4 | F36 |
| 106 | 1-1 | F4 | F37 |
| 107 | 1-1 | F4 | F38 |
| 108 | 1-1 | F4 | F39 |
| 109 | 1-1 | F4 | F40 |
| 110 | 1-1 | F4 | F42 |

TABLE 4

| Compound NO. | Chemical structure | Ar₁ | Ar₂ |
|---|---|---|---|
| 111 | 1-1 | F5 | F5 |
| 112 | 1-1 | F5 | F6 |
| 113 | 1-1 | F5 | F7 |
| 114 | 1-1 | F5 | F9 |
| 115 | 1-1 | F5 | F10 |
| 116 | 1-1 | F5 | F11 |
| 117 | 1-1 | F5 | F12 |
| 118 | 1-1 | F5 | F13 |
| 119 | 1-1 | F5 | F14 |
| 120 | 1-1 | F5 | F29 |
| 121 | 1-1 | F5 | F31 |
| 122 | 1-1 | F5 | F32 |
| 123 | 1-1 | F5 | F33 |
| 124 | 1-1 | F5 | F34 |
| 125 | 1-1 | F5 | F35 |
| 126 | 1-1 | F5 | F36 |
| 127 | 1-1 | F5 | F37 |
| 128 | 1-1 | F5 | F38 |
| 129 | 1-1 | F5 | F39 |
| 130 | 1-1 | F5 | F40 |
| 131 | 1-1 | F5 | F42 |
| 132 | 1-1 | F6 | F7 |
| 133 | 1-1 | F6 | F9 |
| 134 | 1-1 | F6 | F10 |
| 135 | 1-1 | F6 | F11 |
| 136 | 1-1 | F6 | F12 |
| 137 | 1-1 | F6 | F13 |
| 138 | 1-1 | F6 | F14 |
| 139 | 1-1 | F6 | F29 |
| 140 | 1-1 | F6 | F32 |
| 141 | 1-1 | F6 | F33 |
| 142 | 1-1 | F6 | F34 |
| 143 | 1-1 | F6 | F35 |
| 144 | 1-1 | F6 | F36 |
| 145 | 1-1 | F6 | F37 |
| 146 | 1-1 | F6 | F38 |
| 147 | 1-1 | F6 | F39 |
| 148 | 1-1 | F6 | F40 |
| 149 | 1-1 | F6 | F42 |
| 150 | 1-1 | F7 | F10 |
| 151 | 1-1 | F7 | F11 |
| 152 | 1-1 | F7 | F13 |
| 153 | 1-1 | F7 | F14 |
| 154 | 1-1 | F7 | F29 |
| 155 | 1-1 | F7 | F32 |
| 156 | 1-1 | F7 | F33 |
| 157 | 1-1 | F7 | F34 |
| 158 | 1-1 | F7 | F35 |
| 159 | 1-1 | F7 | F36 |
| 160 | 1-1 | F7 | F37 |
| 161 | 1-1 | F7 | F38 |
| 162 | 1-1 | F7 | F39 |
| 163 | 1-1 | F7 | F40 |
| 164 | 1-1 | F7 | F42 |
| 165 | 1-1 | F9 | F10 |
| 166 | 1-1 | F9 | F11 |
| 167 | 1-1 | F9 | F12 |
| 168 | 1-1 | F9 | F13 |
| 169 | 1-1 | F9 | F14 |
| 170 | 1-1 | F9 | F29 |
| 171 | 1-1 | F9 | F32 |
| 172 | 1-1 | F9 | F33 |
| 173 | 1-1 | F9 | F34 |
| 174 | 1-1 | F9 | F35 |
| 175 | 1-1 | F9 | F36 |
| 176 | 1-1 | F9 | F37 |
| 177 | 1-1 | F9 | F38 |
| 178 | 1-1 | F9 | F39 |
| 179 | 1-1 | F9 | F40 |
| 180 | 1-1 | F9 | F42 |
| 181 | 1-1 | F10 | F10 |
| 182 | 1-1 | F10 | F11 |
| 183 | 1-1 | F10 | F12 |
| 184 | 1-1 | F10 | F13 |
| 185 | 1-1 | F10 | F14 |
| 186 | 1-1 | F10 | F29 |
| 187 | 1-1 | F10 | F31 |
| 188 | 1-1 | F10 | F32 |
| 189 | 1-1 | F10 | F33 |
| 190 | 1-1 | F10 | F34 |
| 191 | 1-1 | F10 | F35 |
| 192 | 1-1 | F10 | F36 |
| 193 | 1-1 | F10 | F37 |
| 194 | 1-1 | F10 | F38 |
| 195 | 1-1 | F10 | F39 |
| 196 | 1-1 | F10 | F40 |
| 197 | 1-1 | F10 | F42 |
| 198 | 1-1 | F11 | F11 |
| 199 | 1-1 | F11 | F12 |
| 200 | 1-1 | F11 | F13 |

TABLE 5

| Compound NO. | Chemical structure | Ar₁ | Ar₂ |
|---|---|---|---|
| 201 | 1-1 | F11 | F14 |
| 202 | 1-1 | F11 | F29 |
| 203 | 1-1 | F11 | F31 |
| 204 | 1-1 | F11 | F32 |
| 205 | 1-1 | F11 | F33 |
| 206 | 1-1 | F11 | F34 |
| 207 | 1-1 | F11 | F35 |
| 208 | 1-1 | F11 | F36 |
| 209 | 1-1 | F11 | F37 |
| 210 | 1-1 | F11 | F38 |
| 211 | 1-1 | F11 | F39 |
| 212 | 1-1 | F11 | F40 |
| 213 | 1-1 | F11 | F42 |
| 214 | 1-1 | F12 | F13 |
| 215 | 1-1 | F12 | F14 |
| 216 | 1-1 | F12 | F29 |
| 217 | 1-1 | F12 | F31 |
| 218 | 1-1 | F12 | F32 |
| 219 | 1-1 | F12 | F33 |
| 220 | 1-1 | F12 | F34 |
| 221 | 1-1 | F12 | F35 |
| 222 | 1-1 | F12 | F36 |
| 223 | 1-1 | F12 | F37 |
| 224 | 1-1 | F12 | F38 |
| 225 | 1-1 | F12 | F39 |
| 226 | 1-1 | F12 | F40 |
| 227 | 1-1 | F12 | F42 |
| 228 | 1-1 | F13 | F13 |
| 229 | 1-1 | F13 | F14 |
| 230 | 1-1 | F13 | F29 |
| 231 | 1-1 | F13 | F31 |
| 232 | 1-1 | F13 | F32 |
| 233 | 1-1 | F13 | F33 |
| 234 | 1-1 | F13 | F34 |
| 235 | 1-1 | F13 | F35 |
| 236 | 1-1 | F13 | F36 |
| 237 | 1-1 | F13 | F37 |
| 238 | 1-1 | F13 | F38 |
| 239 | 1-1 | F13 | F39 |
| 240 | 1-1 | F13 | F40 |
| 241 | 1-1 | F13 | F42 |
| 242 | 1-1 | F14 | F14 |
| 243 | 1-1 | F14 | F29 |
| 244 | 1-1 | F14 | F31 |
| 245 | 1-1 | F14 | F32 |
| 246 | 1-1 | F14 | F33 |
| 247 | 1-1 | F14 | F34 |
| 248 | 1-1 | F14 | F35 |
| 249 | 1-1 | F14 | F36 |
| 250 | 1-1 | F14 | F37 |
| 251 | 1-1 | F14 | F38 |
| 252 | 1-1 | F14 | F39 |
| 253 | 1-1 | F14 | F40 |
| 254 | 1-1 | F14 | F42 |
| 255 | 1-1 | F29 | F29 |
| 256 | 1-1 | F29 | F31 |
| 257 | 1-1 | F29 | F32 |
| 258 | 1-1 | F29 | F33 |
| 259 | 1-1 | F29 | F34 |
| 260 | 1-1 | F29 | F35 |
| 261 | 1-1 | F29 | F36 |
| 262 | 1-1 | F29 | F37 |
| 263 | 1-1 | F29 | F38 |
| 264 | 1-1 | F29 | F39 |
| 265 | 1-1 | F29 | F40 |
| 266 | 1-1 | F29 | F42 |
| 267 | 1-1 | F31 | F31 |
| 268 | 1-1 | F31 | F32 |
| 269 | 1-1 | F31 | F34 |
| 270 | 1-1 | F31 | F35 |
| 271 | 1-1 | F31 | F36 |
| 272 | 1-1 | F31 | F37 |
| 273 | 1-1 | F31 | F38 |
| 274 | 1-1 | F31 | F39 |
| 275 | 1-1 | F31 | F40 |
| 276 | 1-1 | F31 | F42 |
| 277 | 1-1 | F32 | F32 |
| 278 | 1-1 | F32 | F33 |
| 279 | 1-1 | F32 | F34 |
| 280 | 1-1 | F32 | F35 |
| 281 | 1-1 | F32 | F36 |
| 282 | 1-1 | F32 | F37 |
| 283 | 1-1 | F32 | F38 |
| 284 | 1-1 | F32 | F39 |
| 285 | 1-1 | F32 | F40 |
| 286 | 1-1 | F32 | F42 |
| 287 | 1-1 | F33 | F33 |
| 288 | 1-1 | F33 | F34 |
| 289 | 1-1 | F33 | F35 |
| 290 | 1-1 | F33 | F36 |

TABLE 6

| Compound NO. | Chemical structure | Ar₁ | Ar₂ |
|---|---|---|---|
| 291 | 1-1 | F33 | F37 |
| 292 | 1-1 | F33 | F38 |
| 293 | 1-1 | F33 | F39 |
| 294 | 1-1 | F33 | F40 |
| 295 | 1-1 | F33 | F42 |
| 296 | 1-1 | F34 | F34 |
| 297 | 1-1 | F34 | F35 |
| 298 | 1-1 | F34 | F36 |
| 299 | 1-1 | F34 | F37 |
| 300 | 1-1 | F34 | F38 |
| 301 | 1-1 | F34 | F39 |
| 302 | 1-1 | F34 | F40 |
| 303 | 1-1 | F34 | F42 |
| 304 | 1-1 | F35 | F35 |
| 305 | 1-1 | F35 | F36 |
| 306 | 1-1 | F35 | F37 |
| 307 | 1-1 | F35 | F38 |
| 308 | 1-1 | F35 | F39 |
| 309 | 1-1 | F35 | F40 |
| 310 | 1-1 | F35 | F42 |
| 311 | 1-1 | F36 | F36 |
| 312 | 1-1 | F36 | F37 |
| 313 | 1-1 | F36 | F38 |
| 314 | 1-1 | F36 | F39 |
| 315 | 1-1 | F36 | F40 |
| 316 | 1-1 | F36 | F42 |
| 317 | 1-1 | F37 | F37 |
| 318 | 1-1 | F37 | F38 |
| 319 | 1-1 | F37 | F39 |
| 320 | 1-1 | F37 | F40 |
| 321 | 1-1 | F37 | F42 |
| 322 | 1-1 | F38 | F38 |
| 323 | 1-1 | F38 | F39 |
| 324 | 1-1 | F38 | F40 |
| 325 | 1-1 | F38 | F42 |
| 326 | 1-1 | F39 | F39 |
| 327 | 1-1 | F39 | F40 |
| 328 | 1-1 | F39 | F42 |
| 329 | 1-1 | F40 | F40 |
| 330 | 1-1 | F40 | F42 |
| 331 | 1-1 | F42 | F42 |
| 332 | 1-2 | F1 | F1 |
| 333 | 1-2 | F3 | F4 |
| 334 | 1-2 | F9 | F9 |
| 335 | 1-2 | F12 | F12 |
| 336 | 1-2 | F29 | F29 |
| 337 | 1-2 | F37 | F37 |
| 338 | 1-3 | F1 | F1 |
| 339 | 1-3 | F1 | F2 |
| 340 | 1-3 | F1 | F3 |
| 341 | 1-3 | F1 | F4 |
| 342 | 1-3 | F1 | F5 |
| 343 | 1-3 | F1 | F6 |
| 344 | 1-3 | F1 | F7 |
| 345 | 1-3 | F1 | F9 |
| 346 | 1-3 | F1 | F10 |
| 347 | 1-3 | F1 | F11 |
| 348 | 1-3 | F1 | F12 |
| 349 | 1-3 | F1 | F13 |
| 350 | 1-3 | F1 | F14 |
| 351 | 1-3 | F1 | F29 |
| 352 | 1-3 | F1 | F31 |
| 353 | 1-3 | F1 | F32 |
| 354 | 1-3 | F1 | F33 |
| 355 | 1-3 | F1 | F34 |
| 356 | 1-3 | F1 | F35 |
| 357 | 1-3 | F1 | F36 |
| 358 | 1-3 | F1 | F37 |
| 359 | 1-3 | F1 | F38 |
| 360 | 1-3 | F1 | F39 |
| 361 | 1-3 | F1 | F40 |
| 362 | 1-3 | F1 | F42 |
| 363 | 1-3 | F2 | F2 |
| 364 | 1-3 | F2 | F3 |
| 365 | 1-3 | F2 | F4 |
| 366 | 1-3 | F2 | F5 |
| 367 | 1-3 | F2 | F6 |
| 368 | 1-3 | F2 | F7 |
| 369 | 1-3 | F2 | F9 |
| 370 | 1-3 | F2 | F10 |
| 371 | 1-3 | F2 | F11 |
| 372 | 1-3 | F2 | F12 |
| 373 | 1-3 | F2 | F13 |
| 374 | 1-3 | F2 | F14 |
| 375 | 1-3 | F2 | F29 |
| 376 | 1-3 | F2 | F31 |
| 377 | 1-3 | F2 | F32 |
| 378 | 1-3 | F2 | F33 |
| 379 | 1-3 | F2 | F34 |
| 380 | 1-3 | F2 | F35 |

TABLE 7

| Compound NO. | Chemical structure | Ar₁ | Ar₂ |
|---|---|---|---|
| 381 | 1-3 | F2 | F36 |
| 382 | 1-3 | F2 | F37 |
| 383 | 1-3 | F2 | F38 |
| 384 | 1-3 | F2 | F39 |
| 385 | 1-3 | F2 | F40 |
| 386 | 1-3 | F2 | F42 |
| 387 | 1-3 | F3 | F3 |
| 388 | 1-3 | F3 | F4 |
| 389 | 1-3 | F3 | F5 |
| 390 | 1-3 | F3 | F6 |
| 391 | 1-3 | F3 | F7 |
| 392 | 1-3 | F3 | F9 |
| 393 | 1-3 | F3 | F10 |
| 394 | 1-3 | F3 | F11 |
| 395 | 1-3 | F3 | F12 |
| 396 | 1-3 | F3 | F13 |
| 397 | 1-3 | F3 | F14 |
| 398 | 1-3 | F3 | F29 |
| 399 | 1-3 | F3 | F31 |
| 400 | 1-3 | F3 | F32 |
| 401 | 1-3 | F3 | F33 |
| 402 | 1-3 | F3 | F34 |
| 403 | 1-3 | F3 | F35 |
| 404 | 1-3 | F3 | F36 |
| 405 | 1-3 | F3 | F37 |
| 406 | 1-3 | F3 | F38 |
| 407 | 1-3 | F3 | F39 |
| 408 | 1-3 | F3 | F40 |
| 409 | 1-3 | F3 | F42 |
| 410 | 1-3 | F4 | F4 |
| 411 | 1-3 | F4 | F5 |
| 412 | 1-3 | F4 | F6 |
| 413 | 1-3 | F4 | F7 |
| 414 | 1-3 | F4 | F9 |
| 415 | 1-3 | F4 | F10 |
| 416 | 1-3 | F4 | F11 |
| 417 | 1-3 | F4 | F12 |
| 418 | 1-3 | F4 | F13 |
| 419 | 1-3 | F4 | F14 |
| 420 | 1-3 | F4 | F29 |
| 421 | 1-3 | F4 | F31 |
| 422 | 1-3 | F4 | F32 |
| 423 | 1-3 | F4 | F33 |
| 424 | 1-3 | F4 | F34 |
| 425 | 1-3 | F4 | F35 |
| 426 | 1-3 | F4 | F36 |
| 427 | 1-3 | F4 | F37 |
| 428 | 1-3 | F4 | F38 |
| 429 | 1-3 | F4 | F39 |
| 430 | 1-3 | F4 | F40 |
| 431 | 1-3 | F4 | F42 |
| 432 | 1-3 | F5 | F5 |
| 433 | 1-3 | F5 | F6 |
| 434 | 1-3 | F5 | F7 |
| 435 | 1-3 | F5 | F9 |
| 436 | 1-3 | F5 | F10 |
| 437 | 1-3 | F5 | F11 |
| 438 | 1-3 | F5 | F12 |
| 439 | 1-3 | F5 | F13 |
| 440 | 1-3 | F5 | F14 |
| 441 | 1-3 | F5 | F29 |
| 442 | 1-3 | F5 | F31 |
| 443 | 1-3 | F5 | F32 |
| 444 | 1-3 | F5 | F33 |
| 445 | 1-3 | F5 | F34 |
| 446 | 1-3 | F5 | F35 |
| 447 | 1-3 | F5 | F36 |
| 448 | 1-3 | F5 | F37 |
| 449 | 1-3 | F5 | F38 |
| 450 | 1-3 | F5 | F39 |
| 451 | 1-3 | F5 | F40 |
| 452 | 1-3 | F5 | F42 |
| 453 | 1-3 | F6 | F6 |
| 454 | 1-3 | F6 | F7 |
| 455 | 1-3 | F6 | F9 |
| 456 | 1-3 | F6 | F10 |
| 457 | 1-3 | F6 | F11 |
| 458 | 1-3 | F6 | F12 |
| 459 | 1-3 | F6 | F13 |
| 460 | 1-3 | F6 | F14 |
| 461 | 1-3 | F6 | F29 |
| 462 | 1-3 | F6 | F31 |
| 463 | 1-3 | F6 | F32 |
| 464 | 1-3 | F6 | F33 |
| 465 | 1-3 | F6 | F34 |
| 466 | 1-3 | F6 | F35 |
| 467 | 1-3 | F6 | F36 |
| 468 | 1-3 | F6 | F37 |
| 469 | 1-3 | F6 | F38 |
| 470 | 1-3 | F6 | F39 |

TABLE 8

| Compound NO. | Chemical structure | Ar₁ | Ar₂ |
|---|---|---|---|
| 471 | 1-3 | F6 | F40 |
| 472 | 1-3 | F6 | F42 |
| 473 | 1-3 | F7 | F7 |
| 474 | 1-3 | F7 | F9 |
| 475 | 1-3 | F7 | F10 |
| 476 | 1-3 | F7 | F11 |
| 477 | 1-3 | F7 | F12 |
| 478 | 1-3 | F7 | F13 |
| 479 | 1-3 | F7 | F14 |
| 480 | 1-3 | F7 | F29 |
| 481 | 1-3 | F7 | F31 |
| 482 | 1-3 | F7 | F32 |
| 483 | 1-3 | F7 | F33 |
| 484 | 1-3 | F7 | F34 |
| 485 | 1-3 | F7 | F35 |
| 486 | 1-3 | F7 | F36 |
| 487 | 1-3 | F7 | F37 |
| 488 | 1-3 | F7 | F38 |
| 489 | 1-3 | F7 | F39 |
| 490 | 1-3 | F7 | F40 |
| 491 | 1-3 | F7 | F42 |
| 492 | 1-3 | F9 | F9 |
| 493 | 1-3 | F9 | F10 |
| 494 | 1-3 | F9 | F11 |
| 495 | 1-3 | F9 | F12 |
| 496 | 1-3 | F9 | F13 |
| 497 | 1-3 | F9 | F14 |
| 498 | 1-3 | F9 | F29 |
| 499 | 1-3 | F9 | F31 |
| 500 | 1-3 | F9 | F32 |
| 501 | 1-3 | F9 | F33 |
| 502 | 1-3 | F9 | F34 |
| 503 | 1-3 | F9 | F35 |
| 504 | 1-3 | F9 | F36 |
| 505 | 1-3 | F9 | F37 |
| 506 | 1-3 | F9 | F38 |
| 507 | 1-3 | F9 | F39 |
| 508 | 1-3 | F9 | F40 |
| 509 | 1-3 | F9 | F42 |
| 510 | 1-3 | F10 | F10 |
| 511 | 1-3 | F10 | F11 |
| 512 | 1-3 | F10 | F12 |
| 513 | 1-3 | F10 | F13 |
| 514 | 1-3 | F10 | F14 |
| 515 | 1-3 | F10 | F29 |
| 516 | 1-3 | F10 | F31 |
| 517 | 1-3 | F10 | F32 |
| 518 | 1-3 | F10 | F33 |
| 519 | 1-3 | F10 | F34 |
| 520 | 1-3 | F10 | F35 |
| 521 | 1-3 | F10 | F36 |
| 522 | 1-3 | F10 | F37 |
| 523 | 1-3 | F10 | F38 |
| 524 | 1-3 | F10 | F39 |
| 525 | 1-3 | F10 | F40 |
| 526 | 1-3 | F10 | F42 |
| 527 | 1-3 | F11 | F11 |
| 528 | 1-3 | F11 | F12 |
| 529 | 1-3 | F11 | F13 |
| 530 | 1-3 | F11 | F14 |
| 531 | 1-3 | F11 | F29 |
| 532 | 1-3 | F11 | F31 |
| 533 | 1-3 | F11 | F32 |
| 534 | 1-3 | F11 | F33 |
| 535 | 1-3 | F11 | F34 |
| 536 | 1-3 | F11 | F35 |
| 537 | 1-3 | F11 | F36 |
| 538 | 1-3 | F11 | F37 |
| 539 | 1-3 | F11 | F38 |
| 540 | 1-3 | F11 | F39 |
| 541 | 1-3 | F11 | F40 |
| 542 | 1-3 | F11 | F42 |
| 543 | 1-3 | F12 | F12 |
| 544 | 1-3 | F12 | F13 |
| 545 | 1-3 | F12 | F14 |
| 546 | 1-3 | F12 | F29 |
| 547 | 1-3 | F12 | F31 |
| 548 | 1-3 | F12 | F32 |
| 549 | 1-3 | F12 | F33 |
| 550 | 1-3 | F12 | F34 |
| 551 | 1-3 | F12 | F35 |
| 552 | 1-3 | F12 | F36 |
| 553 | 1-3 | F12 | F37 |
| 554 | 1-3 | F12 | F38 |
| 555 | 1-3 | F12 | F39 |
| 556 | 1-3 | F12 | F40 |
| 557 | 1-3 | F12 | F42 |
| 558 | 1-3 | F13 | F13 |
| 559 | 1-3 | F13 | F14 |
| 560 | 1-3 | F13 | F29 |

TABLE 9

| Compound NO. | Chemical structure | Ar₁ | Ar₂ |
|---|---|---|---|
| 561 | 1-3 | F13 | F31 |
| 562 | 1-3 | F13 | F32 |
| 563 | 1-3 | F13 | F33 |
| 564 | 1-3 | F13 | F34 |
| 565 | 1-3 | F13 | F35 |
| 566 | 1-3 | F13 | F36 |
| 567 | 1-3 | F13 | F37 |
| 568 | 1-3 | F13 | F38 |
| 569 | 1-3 | F13 | F39 |
| 570 | 1-3 | F13 | F40 |
| 571 | 1-3 | F13 | F42 |
| 572 | 1-3 | F14 | F14 |
| 573 | 1-3 | F14 | F29 |
| 574 | 1-3 | F14 | F31 |
| 575 | 1-3 | F14 | F32 |
| 576 | 1-3 | F14 | F33 |
| 577 | 1-3 | F14 | F34 |
| 578 | 1-3 | F14 | F35 |
| 579 | 1-3 | F14 | F36 |
| 580 | 1-3 | F14 | F37 |
| 581 | 1-3 | F14 | F38 |
| 582 | 1-3 | F14 | F39 |
| 583 | 1-3 | F14 | F40 |
| 584 | 1-3 | F14 | F42 |
| 585 | 1-3 | F29 | F29 |
| 586 | 1-3 | F29 | F31 |
| 587 | 1-3 | F29 | F32 |
| 588 | 1-3 | F29 | F33 |
| 589 | 1-3 | F29 | F34 |
| 590 | 1-3 | F29 | F35 |
| 591 | 1-3 | F29 | F36 |
| 592 | 1-3 | F29 | F37 |
| 593 | 1-3 | F29 | F38 |
| 594 | 1-3 | F29 | F39 |
| 595 | 1-3 | F29 | F40 |
| 596 | 1-3 | F29 | F42 |
| 597 | 1-3 | F31 | F31 |
| 598 | 1-3 | F31 | F32 |
| 599 | 1-3 | F31 | F33 |
| 600 | 1-3 | F31 | F34 |
| 601 | 1-3 | F31 | F35 |
| 602 | 1-3 | F31 | F36 |
| 603 | 1-3 | F31 | F37 |
| 604 | 1-3 | F31 | F38 |
| 605 | 1-3 | F31 | F39 |
| 606 | 1-3 | F31 | F40 |
| 607 | 1-3 | F31 | F42 |
| 608 | 1-3 | F32 | F32 |
| 609 | 1-3 | F32 | F33 |
| 610 | 1-3 | F32 | F34 |
| 611 | 1-3 | F32 | F35 |
| 612 | 1-3 | F32 | F36 |
| 613 | 1-3 | F32 | F37 |
| 614 | 1-3 | F32 | F38 |
| 615 | 1-3 | F32 | F39 |
| 616 | 1-3 | F32 | F40 |
| 617 | 1-3 | F32 | F42 |
| 618 | 1-3 | F33 | F33 |
| 619 | 1-3 | F33 | F34 |
| 620 | 1-3 | F33 | F35 |
| 621 | 1-3 | F33 | F36 |
| 622 | 1-3 | F33 | F37 |
| 623 | 1-3 | F33 | F38 |
| 624 | 1-3 | F33 | F39 |
| 625 | 1-3 | F33 | F40 |
| 626 | 1-3 | F33 | F42 |
| 627 | 1-3 | F34 | F34 |
| 628 | 1-3 | F34 | F35 |
| 629 | 1-3 | F34 | F36 |
| 630 | 1-3 | F34 | F37 |
| 631 | 1-3 | F34 | F38 |
| 632 | 1-3 | F34 | F39 |
| 633 | 1-3 | F34 | F40 |
| 634 | 1-3 | F34 | F42 |
| 635 | 1-3 | F35 | F35 |
| 636 | 1-3 | F35 | F36 |
| 637 | 1-3 | F35 | F37 |
| 638 | 1-3 | F35 | F38 |
| 639 | 1-3 | F35 | F39 |
| 640 | 1-3 | F35 | F40 |
| 641 | 1-3 | F35 | F42 |
| 642 | 1-3 | F36 | F36 |
| 643 | 1-3 | F36 | F37 |
| 644 | 1-3 | F36 | F38 |
| 645 | 1-3 | F36 | F39 |
| 646 | 1-3 | F36 | F40 |
| 647 | 1-3 | F36 | F42 |
| 648 | 1-3 | F37 | F37 |
| 649 | 1-3 | F37 | F38 |
| 650 | 1-3 | F37 | F39 |

TABLE 10

| Compound NO. | Chemical structure | Ar₁ | Ar₂ |
|---|---|---|---|
| 651 | 1-3 | F37 | F40 |
| 652 | 1-3 | F37 | F42 |
| 653 | 1-3 | F38 | F38 |
| 654 | 1-3 | F38 | F39 |
| 655 | 1-3 | F38 | F40 |
| 656 | 1-3 | F38 | F42 |
| 657 | 1-3 | F39 | F39 |
| 658 | 1-3 | F39 | F40 |
| 659 | 1-3 | F39 | F42 |
| 660 | 1-3 | F40 | F40 |
| 661 | 1-3 | F40 | F42 |
| 662 | 1-3 | F42 | F42 |
| 663 | 1-4 | F1 | F36 |
| 664 | 1-4 | F4 | F4 |
| 665 | 1-4 | F5 | F5 |
| 666 | 1-4 | F9 | F31 |
| 667 | 1-4 | F31 | F33 |
| 668 | 1-4 | F35 | F35 |
| 669 | 1-4 | F37 | F37 |
| 670 | 1-5 | F1 | F1 |
| 671 | 1-5 | F1 | F2 |
| 672 | 1-5 | F1 | F3 |
| 673 | 1-5 | F1 | F4 |
| 674 | 1-5 | F1 | F5 |
| 675 | 1-5 | F1 | F6 |
| 676 | 1-5 | F1 | F7 |
| 677 | 1-5 | F1 | F9 |
| 678 | 1-5 | F1 | F10 |
| 679 | 1-5 | F1 | F11 |
| 680 | 1-5 | F1 | F12 |
| 681 | 1-3 | F1 | F13 |
| 682 | 1-3 | F1 | F14 |
| 683 | 1-3 | F1 | F29 |
| 684 | 1-3 | F1 | F31 |
| 685 | 1-3 | F1 | F32 |
| 686 | 1-3 | F1 | F33 |
| 687 | 1-3 | F1 | F34 |
| 688 | 1-3 | F1 | F35 |
| 689 | 1-3 | F1 | F36 |
| 690 | 1-3 | F1 | F37 |
| 691 | 1-3 | F1 | F38 |
| 692 | 1-3 | F1 | F39 |
| 693 | 1-4 | F1 | F40 |
| 694 | 1-4 | F1 | F42 |
| 695 | 1-4 | F2 | F2 |
| 696 | 1-4 | F2 | F3 |
| 697 | 1-4 | F2 | F4 |
| 698 | 1-4 | F2 | F5 |
| 699 | 1-4 | F2 | F6 |
| 700 | 1-5 | F2 | F7 |
| 701 | 1-5 | F2 | F9 |
| 702 | 1-5 | F2 | F10 |
| 703 | 1-5 | F2 | F11 |
| 704 | 1-5 | F2 | F12 |
| 705 | 1-5 | F2 | F13 |
| 706 | 1-5 | F2 | F14 |
| 707 | 1-5 | F2 | F29 |
| 708 | 1-5 | F2 | F31 |
| 709 | 1-5 | F2 | F32 |
| 710 | 1-5 | F2 | F33 |
| 711 | 1-5 | F2 | F34 |
| 712 | 1-5 | F2 | F35 |
| 713 | 1-5 | F2 | F36 |
| 714 | 1-5 | F2 | F37 |
| 715 | 1-5 | F2 | F38 |
| 716 | 1-5 | F2 | F39 |
| 717 | 1-5 | F2 | F40 |
| 718 | 1-5 | F2 | F42 |
| 719 | 1-5 | F3 | F3 |
| 720 | 1-5 | F3 | F4 |
| 721 | 1-5 | F3 | F5 |
| 722 | 1-5 | F3 | F6 |
| 723 | 1-5 | F3 | F7 |
| 724 | 1-5 | F3 | F9 |
| 725 | 1-5 | F3 | F10 |
| 726 | 1-5 | F3 | F11 |
| 727 | 1-5 | F3 | F12 |
| 728 | 1-5 | F3 | F13 |
| 729 | 1-5 | F3 | F14 |
| 730 | 1-5 | F3 | F29 |
| 731 | 1-5 | F3 | F31 |
| 732 | 1-5 | F3 | F32 |
| 733 | 1-5 | F3 | F33 |
| 734 | 1-5 | F3 | F34 |
| 735 | 1-5 | F3 | F35 |
| 736 | 1-5 | F3 | F36 |
| 737 | 1-5 | F3 | F37 |
| 738 | 1-5 | F3 | F38 |
| 739 | 1-5 | F3 | F39 |
| 740 | 1-5 | F3 | F40 |

TABLE 11

| Compound NO. | Chemical structure | Ar₁ | Ar₂ |
|---|---|---|---|
| 741 | 1-5 | F3 | F42 |
| 742 | 1-5 | F4 | F4 |
| 743 | 1-5 | F4 | F5 |
| 744 | 1-5 | F4 | F6 |
| 745 | 1-5 | F4 | F7 |
| 746 | 1-5 | F4 | F9 |
| 747 | 1-5 | F4 | F10 |
| 748 | 1-5 | F4 | F11 |
| 749 | 1-5 | F4 | F12 |
| 750 | 1-5 | F4 | F13 |
| 751 | 1-5 | F4 | F14 |
| 752 | 1-5 | F4 | F29 |
| 753 | 1-5 | F4 | F31 |
| 754 | 1-5 | F4 | F32 |
| 755 | 1-5 | F4 | F33 |
| 756 | 1-5 | F4 | F34 |
| 757 | 1-5 | F4 | F35 |
| 758 | 1-5 | F4 | F36 |
| 759 | 1-5 | F4 | F37 |
| 760 | 1-5 | F4 | F38 |
| 761 | 1-5 | F4 | F39 |
| 762 | 1-5 | F4 | F40 |
| 763 | 1-5 | F4 | F42 |
| 764 | 1-5 | F5 | F5 |
| 765 | 1-5 | F5 | F6 |
| 766 | 1-5 | F5 | F7 |
| 767 | 1-5 | F5 | F9 |
| 768 | 1-5 | F5 | F10 |
| 769 | 1-5 | F5 | F11 |
| 770 | 1-5 | F5 | F12 |
| 771 | 1-5 | F5 | F13 |
| 772 | 1-5 | F5 | F14 |
| 773 | 1-5 | F5 | F29 |
| 774 | 1-5 | F5 | F31 |
| 775 | 1-5 | F5 | F32 |
| 776 | 1-5 | F5 | F33 |
| 777 | 1-5 | F5 | F34 |
| 778 | 1-5 | F5 | F35 |
| 779 | 1-5 | F5 | F36 |
| 780 | 1-5 | F5 | F37 |
| 781 | 1-5 | F5 | F38 |
| 782 | 1-5 | F5 | F39 |
| 783 | 1-5 | F5 | F40 |
| 784 | 1-5 | F5 | F42 |
| 785 | 1-5 | F6 | F6 |
| 786 | 1-5 | F6 | F7 |
| 787 | 1-5 | F6 | F9 |
| 788 | 1-5 | F6 | F10 |
| 789 | 1-5 | F6 | F11 |
| 790 | 1-5 | F6 | F12 |
| 791 | 1-5 | F6 | F13 |
| 792 | 1-5 | F6 | F14 |
| 793 | 1-5 | F6 | F29 |
| 794 | 1-5 | F6 | F31 |
| 795 | 1-5 | F6 | F32 |
| 796 | 1-5 | F6 | F33 |
| 797 | 1-5 | F6 | F34 |
| 798 | 1-5 | F6 | F35 |
| 799 | 1-5 | F6 | F36 |
| 800 | 1-5 | F6 | F37 |
| 801 | 1-5 | F6 | F38 |
| 802 | 1-5 | F6 | F39 |
| 803 | 1-5 | F6 | F40 |
| 804 | 1-5 | F6 | F42 |
| 805 | 1-5 | F7 | F7 |
| 806 | 1-5 | F7 | F9 |
| 807 | 1-5 | F7 | F10 |
| 808 | 1-5 | F7 | F11 |
| 809 | 1-5 | F7 | F12 |
| 810 | 1-5 | F7 | F13 |
| 811 | 1-5 | F7 | F14 |
| 812 | 1-5 | F7 | F29 |
| 813 | 1-5 | F7 | F31 |
| 814 | 1-5 | F7 | F32 |
| 815 | 1-5 | F7 | F33 |
| 816 | 1-5 | F7 | F34 |
| 817 | 1-5 | F7 | F35 |
| 818 | 1-5 | F7 | F36 |
| 819 | 1-5 | F7 | F37 |
| 820 | 1-5 | F7 | F38 |
| 821 | 1-5 | F7 | F39 |
| 822 | 1-5 | F7 | F40 |
| 823 | 1-5 | F7 | F42 |
| 824 | 1-5 | F9 | F9 |
| 825 | 1-5 | F9 | F10 |
| 826 | 1-5 | F9 | F11 |
| 827 | 1-5 | F9 | F12 |
| 828 | 1-5 | F9 | F13 |
| 829 | 1-5 | F9 | F14 |
| 830 | 1-5 | F9 | F29 |

TABLE 12

| Compound NO. | Chemical structure | Ar₁ | Ar₂ |
|---|---|---|---|
| 831 | 1-5 | F9 | F31 |
| 832 | 1-5 | F9 | F32 |
| 833 | 1-5 | F9 | F33 |
| 834 | 1-5 | F9 | F34 |
| 835 | 1-5 | F9 | F35 |
| 836 | 1-5 | F9 | F36 |
| 837 | 1-5 | F9 | F37 |
| 838 | 1-5 | F9 | F38 |
| 839 | 1-5 | F9 | F39 |
| 840 | 1-5 | F9 | F40 |
| 841 | 1-5 | F9 | F42 |
| 842 | 1-5 | F10 | F10 |
| 843 | 1-5 | F10 | F11 |
| 844 | 1-5 | F10 | F12 |
| 845 | 1-5 | F10 | F13 |
| 846 | 1-5 | F10 | F14 |
| 847 | 1-5 | F10 | F29 |
| 848 | 1-5 | F10 | F31 |
| 849 | 1-5 | F10 | F32 |
| 850 | 1-5 | F10 | F33 |
| 851 | 1-5 | F10 | F34 |
| 852 | 1-5 | F10 | F35 |
| 853 | 1-5 | F10 | F36 |
| 854 | 1-5 | F10 | F37 |
| 855 | 1-5 | F10 | F38 |
| 856 | 1-5 | F10 | F39 |
| 857 | 1-5 | F10 | F40 |
| 858 | 1-5 | F10 | F42 |
| 859 | 1-5 | F11 | F11 |
| 860 | 1-5 | F11 | F12 |
| 861 | 1-5 | F11 | F13 |
| 862 | 1-5 | F11 | F14 |
| 863 | 1-5 | F11 | F29 |
| 864 | 1-5 | F11 | F31 |
| 865 | 1-5 | F11 | F32 |
| 866 | 1-5 | F11 | F33 |
| 867 | 1-5 | F11 | F34 |
| 868 | 1-5 | F11 | F35 |
| 869 | 1-5 | F11 | F36 |
| 870 | 1-5 | F11 | F37 |
| 871 | 1-5 | F11 | F38 |
| 872 | 1-5 | F11 | F39 |
| 873 | 1-5 | F11 | F40 |
| 874 | 1-5 | F11 | F42 |
| 875 | 1-5 | F12 | F12 |
| 876 | 1-5 | F12 | F13 |
| 877 | 1-5 | F12 | F14 |
| 878 | 1-5 | F12 | F29 |
| 879 | 1-5 | F12 | F31 |
| 880 | 1-5 | F12 | F32 |
| 881 | 1-5 | F12 | F33 |
| 882 | 1-5 | F12 | F34 |
| 883 | 1-5 | F12 | F35 |
| 884 | 1-5 | F12 | F36 |
| 885 | 1-5 | F12 | F37 |
| 886 | 1-5 | F12 | F38 |
| 887 | 1-5 | F12 | F39 |
| 888 | 1-5 | F12 | F40 |
| 889 | 1-5 | F12 | F42 |
| 890 | 1-5 | F13 | F13 |
| 891 | 1-5 | F13 | F14 |
| 892 | 1-5 | F13 | F29 |
| 893 | 1-5 | F13 | F31 |
| 894 | 1-5 | F13 | F32 |
| 895 | 1-5 | F13 | F33 |
| 896 | 1-5 | F13 | F34 |
| 897 | 1-5 | F13 | F35 |
| 898 | 1-5 | F13 | F36 |
| 899 | 1-5 | F13 | F37 |
| 900 | 1-5 | F13 | F38 |
| 901 | 1-5 | F13 | F39 |
| 902 | 1-5 | F13 | F40 |
| 903 | 1-5 | F13 | F42 |
| 904 | 1-5 | F14 | F14 |
| 905 | 1-5 | F14 | F29 |
| 906 | 1-5 | F14 | F31 |
| 907 | 1-5 | F14 | F32 |
| 908 | 1-5 | F14 | F33 |
| 909 | 1-5 | F14 | F34 |
| 910 | 1-5 | F14 | F35 |
| 911 | 1-5 | F14 | F36 |
| 912 | 1-5 | F14 | F37 |
| 913 | 1-5 | F14 | F38 |
| 914 | 1-5 | F14 | F39 |
| 915 | 1-5 | F14 | F40 |
| 916 | 1-5 | F14 | F42 |
| 917 | 1-5 | F29 | F29 |
| 918 | 1-5 | F29 | F31 |
| 919 | 1-5 | F29 | F32 |
| 920 | 1-5 | F29 | F33 |

TABLE 13

| Compound NO. | Chemical structure | Ar₁ | Ar₂ |
|---|---|---|---|
| 921 | 1-5 | F29 | F34 |
| 922 | 1-5 | F29 | F35 |
| 923 | 1-5 | F29 | F36 |
| 924 | 1-5 | F29 | F37 |
| 925 | 1-5 | F29 | F38 |
| 926 | 1-5 | F29 | F39 |
| 927 | 1-5 | F29 | F40 |
| 928 | 1-5 | F29 | F42 |
| 929 | 1-5 | F31 | F31 |
| 930 | 1-5 | F31 | F32 |
| 931 | 1-5 | F31 | F33 |
| 932 | 1-5 | F31 | F34 |
| 933 | 1-5 | F31 | F35 |
| 934 | 1-5 | F31 | F36 |
| 935 | 1-5 | F31 | F37 |
| 936 | 1-5 | F31 | F38 |
| 937 | 1-5 | F31 | F39 |
| 938 | 1-5 | F31 | F40 |
| 939 | 1-5 | F31 | F42 |
| 940 | 1-5 | F32 | F32 |
| 941 | 1-5 | F32 | F33 |
| 942 | 1-5 | F32 | F34 |
| 943 | 1-5 | F32 | F35 |
| 944 | 1-5 | F32 | F36 |
| 945 | 1-5 | F32 | F37 |
| 946 | 1-5 | F32 | F38 |
| 947 | 1-5 | F32 | F39 |
| 948 | 1-5 | F32 | F40 |
| 949 | 1-5 | F32 | F42 |
| 950 | 1-5 | F33 | F33 |
| 951 | 1-5 | F33 | F34 |
| 952 | 1-5 | F33 | F35 |
| 953 | 1-5 | F33 | F36 |
| 954 | 1-5 | F33 | F37 |
| 955 | 1-5 | F33 | F38 |
| 956 | 1-5 | F33 | F39 |
| 957 | 1-5 | F33 | F40 |
| 958 | 1-5 | F33 | F42 |
| 959 | 1-5 | F34 | F34 |
| 960 | 1-5 | F34 | F35 |
| 961 | 1-5 | F34 | F36 |
| 962 | 1-5 | F34 | F37 |
| 963 | 1-5 | F34 | F38 |
| 964 | 1-5 | F34 | F39 |
| 965 | 1-5 | F34 | F40 |
| 966 | 1-5 | F34 | F42 |
| 967 | 1-5 | F35 | F35 |
| 968 | 1-5 | F35 | F36 |
| 969 | 1-5 | F35 | F37 |
| 970 | 1-5 | F35 | F38 |
| 971 | 1-5 | F35 | F39 |
| 972 | 1-5 | F35 | F40 |
| 973 | 1-5 | F35 | F42 |
| 974 | 1-5 | F36 | F36 |
| 975 | 1-5 | F36 | F37 |
| 976 | 1-5 | F36 | F38 |
| 977 | 1-5 | F36 | F39 |
| 978 | 1-5 | F36 | F40 |
| 979 | 1-5 | F36 | F42 |
| 980 | 1-5 | F37 | F37 |
| 981 | 1-5 | F37 | F38 |
| 982 | 1-5 | F37 | F39 |
| 983 | 1-5 | F37 | F40 |
| 984 | 1-5 | F37 | F42 |
| 985 | 1-5 | F38 | F38 |
| 986 | 1-5 | F38 | F39 |
| 987 | 1-5 | F38 | F40 |
| 988 | 1-5 | F38 | F42 |
| 989 | 1-5 | F39 | F39 |
| 990 | 1-5 | F39 | F40 |
| 991 | 1-5 | F39 | F42 |
| 992 | 1-5 | F40 | F40 |
| 993 | 1-5 | F40 | F42 |
| 994 | 1-5 | F42 | F42 |
| 995 | 1-6 | F1 | F7 |
| 996 | 1-6 | F1 | F34 |
| 997 | 1-6 | F1 | F39 |
| 998 | 1-6 | F2 | F2 |
| 999 | 1-6 | F4 | F34 |
| 1000 | 1-6 | F9 | F31 |
| 1001 | 1-6 | F14 | F14 |
| 1002 | 1-6 | F36 | F36 |
| 1003 | 1-6 | F37 | F37 |

As described above, the organic light-emitting diode according to one embodiment may include a first electrode (positive electrode), a second electrode (negative electrode) facing the first electrode, at least one organic material layer positioned on the inner side of the first electrode and the second electrode, and a capping layer positioned on the outer side of at least one of the first electrode and the second electrode.

The organic material layer may include at least one layer of a hole injection layer, a hole transport layer, an electron blocking layer, a light-emitting layer, a hole blocking layer, an electron transport layer, or an electron injection layer, and may additionally include a charge generating layer, a hole transport auxiliary layer, a light emitting auxiliary layer, an electron transport auxiliary layer, etc.

For example, the organic light-emitting diode may have a structure of a first electrode (positive electrode, anode), a hole injection layer (HIL), a hole transport layer (HTL), an electron blocking layer (EBL), a light-emitting layer (EML), a hole blocking layer (HBL), an electron transport layer (ETL), an electron injection layer (EIL), and a second electrode (negative electrode, cathode), sequentially stacked.

For example, the first electrode may include a material which is transparent and has excellent conductivity, including indium tin oxide (ITO), indium zinc oxide (IZO), tin oxide (SnO₂) and zinc oxide (ZnO).

The compound of the hole injection layer or the hole transport layer is not specifically limited, and may use optional compounds commonly used as the compounds of the hole injection layer or the hole transport layer. Non-limiting examples of the compound of the hole injection layer or the hole transport layer include a phthalocyanine derivative, a porphyrin derivative, a triarylamine derivative and an indolocarbazole derivative. For example, 1,4,5,8,9,11-hexaazatriphenylen-hexacarbonitrile (HAT-CN), copper phthalocyanine (CuPc), 4,4′,4″-tris(3-methylphenylamino)triphenylamine (m-MTDATA), 4,4′,4″-tris(3-methylphenylamino) phenoxybenzene (m-MTDAPB), 4,4′,4″-tris(N-carbazolyl)triphenylamine (TCTA), 4,4′,4″-tris(N-(2-naphthyl)-N-phenylamino)-triphenylamine (2-TNATA), N4,N4,N4′,N4′-tetra([1,1′-biphenyl]-4-yl)-[1,1′-biphenyl]-4,4′-diamine, bis(N-(1-naphthyl-n-phenyl))benzidine (α-NPD), N,N′-di(naphthalen-1-yl)-N,N′-biphenyl-benzidine (NPB), N,N′-biphenyl-N,N′-bis(3-methylphenyl)-1,1′-biphenyl-4,4′-diamine (TPD), etc.

The compound included in the light-emitting layer is not specifically limited, and optional compounds used as the common compounds of the light-emitting layer may be used. A single light-emitting compound or a light-emitting host compound may be used.

Here, the light-emitting compound of the light-emitting layer may include compounds which may emit light through phosphorescence, fluorescence, thermally activated delayed fluorescence, i.e., TADF (or referred to as E-type delayed fluorescence), triplet-triplet annihilation, or combinations thereof, without limitation. The light-emitting compound may be selected from various materials according to desired emission color. Non-limiting examples of the light-emitting compound may include a fused ring derivative such as phenanthrene, anthracene, pyrene, tetracene, pentacene, perylene, naphthopyrene, dibenzopyrene, rubrene and chrysene, a benzoxazole derivative, a benzothiazole derivative, a benzoimidazole derivative, a benzotriazole derivative, an oxazole derivative, an oxadiazole derivative, a thiazole derivative, an imidazole derivative, a thiadiazole derivative, a triazole derivative, a pyrazoline derivative, a stilbene derivative, a thiophene derivative, a tetraphenylbutadiene derivative, a cyclopentadiene derivative, a bisstyryl derivative, a bisstyrylarylene derivative, a diazaindacene derivative, a furan derivative, a benzofuran derivative, an isobenzofuran derivative, a dibenzofuran derivative, a coumarine derivative, a dicyanomethylenepyran derivative, a dicyanomethylenethiopyran derivative, a polymethine derivative, a cyanine derivative, an oxobenzoanthracene derivative, a xanthene derivative, a rhodamine derivative, a fluorescein derivative, a pyrylium derivative, a carbostyryl derivative, an acridine derivative, an oxazine derivative, a phenylene oxide derivative, a quinacridone derivative, a quinazoline derivative, a pyrrolopyridine derivative, a furopyridine derivative, a 1,2,5-thiadiazolopyrene derivative, a pyrromethene derivative, a perinone derivative, a pyrrolopyrrole derivative, a squarylium derivative, a bioranthrone derivative, a phenazine derivative, an acridone derivative, a deazaflavin derivative, a fluorene derivative, a benzofluorene derivative, an aromatic boron derivative, an aromatic nitrogen boron derivative, a metal complex (complex of a metal such as Ir, Pt, Au, Eu, Ru, Re, Ag and Cu with a heteroaromatic ring ligand), etc. For example, N1,N1,N6,N6-tetrakis(4-(1-silyl)phenyl) pyren-1,6-diamine, 2,12-di-tert-butyl-5,9-bis(4-(tert-butyl)phenyl)-7-(3,5-di-tert-butylphenyl)-5,9-dihydro-5,9-diaza-13b-borannaphtho[3,2,1-de]anthracene (t-DABNA-dtB), PtOEP, Ir(ppy)₃, Ir(ppy)₂(acac), Ir(mppy)₃, Ir(PPy)₂(m-bppy), BtpIr(acac), Ir(btp)₂(acac), Ir(2-phq)₃, Hex-Ir(phq)₃, Ir(fbi)₂(acac), fac-tris(2-(3-p-xylyl)phenyl)pyridine iridium(III), Eu(dbm)₃(Phen), Ir(piq)₃, Ir(piq)₂(acac), Ir(Fliq)₂(acac), Ir(Flq)₂(acac), Ru(dtb-bpy)₃·2(PF₆), Ir(BT)₂(acac), Ir(DMP)₃, Ir(Mphq)₃, IR(phq)₂tpy, fac-Ir(ppy)₂Pc, Ir(dp)PQ₂, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, etc., may be used.

The host compound of the light-emitting layer may use an emissive host, a hole transport host, an electron transport host, or combinations thereof. Non-limiting examples of the emissive host compound may include a fused ring derivative such as anthracene and pyrene, a bisstyryl derivative such as a bisstyryl anthracene derivative and a distyryl benzene derivative, a tetraphenylbutadiene derivative, a cyclopentadiene derivative, a fluorene derivative, a benzofluorene derivative, a N-phenylcarbazole derivative, a carbazonitrile derivative, etc. Non-limiting examples of the hole transport host material may include a carbazole derivative, a dibenzofuran derivative, a dibenzothiophene derivative, a triarylamine derivative, an indolocarbazole derivative and a benzoxazinophenoxazine derivative. Non-limiting examples of the electron transport host material may include a pyridine derivative, a triazine derivative, a phosphine oxide derivative, benzofuropyridine derivative, and a dibenzooxasiline derivative. For example, 9,10-bis(2-naphthyl)anthracene (ADN), tris(8-hydroxyquinolinato)aluminum (Alq₃), 8-hydroxyquinolineberyllium salt (BAlq), 4,4′-bis(2,2-biphneylethenyl)-1,1′-biphenyl series (DPVBi), spiro-4,4′-bis(2,2-biphenylethenyl)-1,1′-biphenyl (spiro-DPVBi), 2-(2-benzooxazolyl)-phenollithium salt (LiPBO), bis(biphenylvinyl)benzene, an aluminum-quinoline metal complex, metal complexes of imidazole, thiazole and oxazole, etc. may be included.

An electron blocking layer (EBL) may be formed between the hole transport layer and the light-emitting layer. The compound of the electron blocking layer is not specifically limited and may use optional compounds commonly used as the compounds of the electron blocking layer. For example, the electron blocking layer may include N-phenyl-N-(4-(spiro[benzo[d,e]anthracen-7,9′-fluorene]-2′-yl)phenyl)dibenzo[b,d]furan-4-amine), etc.

The compound of the electron injection layer or the electron transport layer is not specifically limited, and optional compounds commonly used as the compounds of the electron injection layer or the electron transport layer may be used. Non-limiting examples of the compound of the electron injection layer or the electron transport layer may include a pyridine derivative, a naphthalene derivative, an anthracene derivative, a phenanthroline derivative, a perinone derivative, a coumarine derivative, a naphthalimide derivative, an anthraquinone derivative, a diphenoquinone derivative, a diphenylquinone derivative, a perylene derivative, an oxadiazole derivative, a thiophene derivative, a triazole derivative, a thiadiazole derivative, a metal complex of an oxine derivative, a quinolinol-based metal complex, a quinoxaline derivative, a polymer of a quinoxaline derivative, benzazole compounds, a gallium complex, a pyrazole derivative, a pefluorinated phenylene derivative, a triazine derivative, a pyrazine derivative, a benzoquinoline derivative, an imidazopyridine derivative, a boran derivative, a benzoimidazole derivative, a benzoxazole derivative, a benzothiazole derivative, a quinoline derivative, an oligo pyridine derivative such as tert-pyridine, a bipyridine derivative, a tert-pyridine derivative, a naphthyridine derivative, an aldazine derivative, a carbazole derivative, an indole derivative, a phosphine oxide derivative, a bisstyryl derivative, a quinolinol-based metal complex, a hydroxyazole-based metal complex, an azomethine-based metal complex, a tropolone-based metal complex, a flavonol-based metal complex, a benzoquinoline-based metal complex, a metal salt, etc. The materials may be used solely, and may be used as a mixture with other materials. For example, a material like 2-(4-(9,10-di(naphthalen-2-yl)anthracen-2-yl)phenyl)-1-phenyl-1H-benzo[d]imidazole, tris(8-hyroxyquinolinato)aluminum (Alq₃), LiF, Liq, Li₂O, BaO, NaCl, CsF, etc. may be included.

An electron transport auxiliary layer may be formed between the electron transport layer and the light-emitting layer. The electron transport auxiliary layer compound is not particularly limited, and any compound that is commonly used as an electron transport auxiliary layer compound may be used. For example, the electron transport auxiliary layer may include a pyrimidine derivative, etc.

The second electrode (negative electrode, cathode) may include a material including lithium (Li), aluminum (Al), aluminum-lithium (Al—Li), calcium (Ca), magnesium (Mg), magnesium-indium (Mg—In), magnesium-silver (Mg—Ag), etc. In addition, in the case of a top emission type organic light-emitting diode, a transparent cathode which may transmit light may be formed using indium tin oxide (ITO) or indium zinc oxide (IZO).

The organic light-emitting diode according to one embodiment of the present disclosure may be a top emission type or a bottom emission type.

The thickness of the capping layer of the organic light-emitting diode according to one embodiment of the present disclosure may be about 300 to 1500 Å, or about 500 to 1200 Å or about 600 to 1000 Å.

The density of the capping layer in the organic light-emitting diode according to one embodiment of the present disclosure may be about 1.15 to 1.35 g/cm³, or about 1.2 to 1.3 g/cm³. Within such a density range, the efficiency of the diode may be improved even further.

The organic light-emitting diode according to one embodiment of the present disclosure may be used in a display device.

The organic light-emitting diode according to one embodiment of the present disclosure may be applied in a transparent display device, a mobile display device, a flexible display device, etc., without limitation. The capping layer according to one embodiment exhibits high transmittance that is suitable for a transparent display device and has high tensile strength that is suitable for a flexible display device.

Hereinafter, typical synthesis methods of the compounds will be explained for illustrations. However, the synthesis method of the compounds of the present disclosure is not limited thereto.

### SYNTHESIS EXAMPLES

The compound of the present disclosure may be synthesized as follows, but is not limited thereto.

Typically, a general reaction formula and a synthesis example for Compound 1 are described, and the compounds represented by Chemical Formula 1 of the present disclosure may be synthesized similarly to the reaction of Compound 1.

A, Ar₁, Ar₂, and n described in the general reaction formula below are as defined in Chemical Formula 1 above.

In the reaction formula below, an amine group (primary group, secondary amine group, etc.) or a halogen group (Br, Cl, etc.) may be any substituent (for example, boron compound, etc.) that may be used in the exemplary catalytic reaction below.

In the reaction formula below, the solvent, catalyst, etc. are typical examples, and all equivalent solvents, catalysts, etc. may be used.

### [General Reaction Formula]

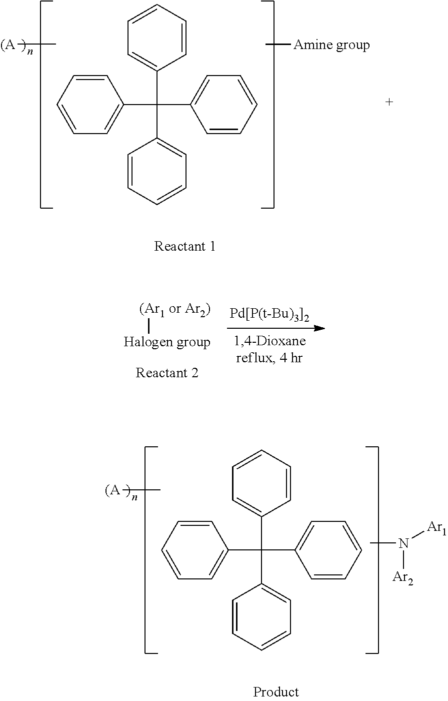

### Synthesis Example 1-Synthesis of Compound 1

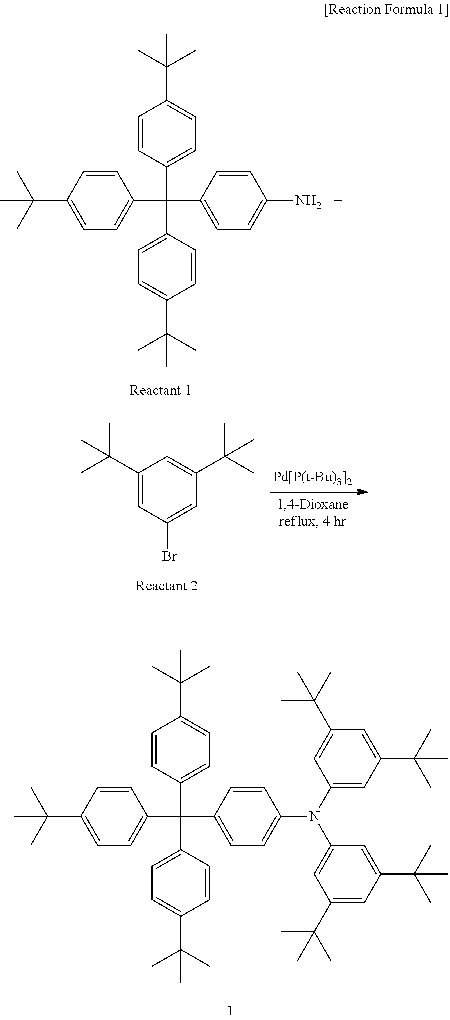

Under a nitrogen atmosphere, Reactant 1 (20 mmol, 10.1 g), Reactant 2 (40 mmol, 10.8 g) and Pd[P(t-Bu)₃]₂ (1.0 mmol, 0.051 g) were added to a 500 mL flask, 1,4-dioxane (200 mL) was added thereto, and the mixture was stirred under reflux conditions for 4 hours. After completion of the reaction, an organic layer was extracted using CH₂Cl₂ and water. The extracted solution was treated with MgSO₄ to remove residual moisture, concentrated under reduced pressure, and purified using a column chromatography method, and then recrystallized to obtain Compound 1.

The results of the synthesis of compounds including Compound 1 are shown in Table 14 below.

TABLE 14

| | | | | Obtained | |
|---|---|---|---|---|---|
| Com- | | | | amount | |
| pound | Reactant 1 | Reactant 2 | Product | (yield) | [M + H]⁺ |
| 1 | 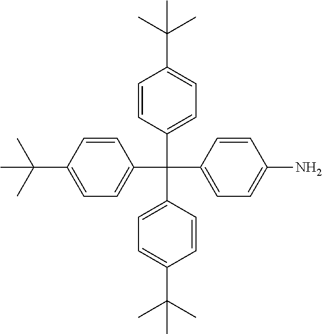 [SMILES: CC(C)(C)c1ccc(C(c2ccc(N)cc2)(c2ccc(C(C)(C)C)cc2)c2ccc(C(C)(C)C)cc2)cc1] | 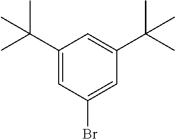 | 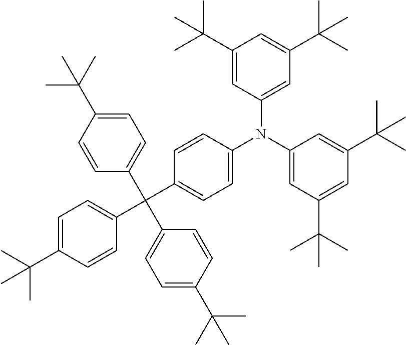 [SMILES: CC(C)(C)c1ccc(C(c2ccc(N(c3cc(C(C)(C)C)cc(C(C)(C)C)c3)c3cc(C(C)(C)C)cc(C(C)(C)C)c3)cc2)(c2ccc(C(C)(C)C)cc2)c2ccc(C(C)(C)C)cc2)cc1] | 15.7 g (89%) | 879.67 |
| 5 | 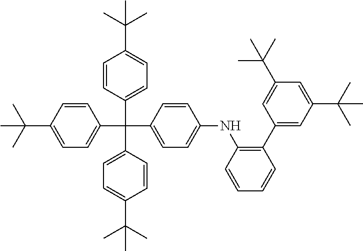 [SMILES: CC(C)(C)c1ccc(C(c2ccc(Nc3ccccc3-c3cc(C(C)(C)C)cc(C(C)(C)C)c3)cc2)(c2ccc(C(C)(C)C)cc2)c2ccc(C(C)(C)C)cc2)cc1] | 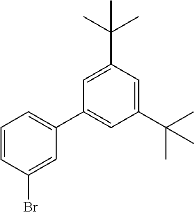 [SMILES: CC(C)(C)c1cc(-c2cccc(Br)c2)cc(C(C)(C)C)c1] | 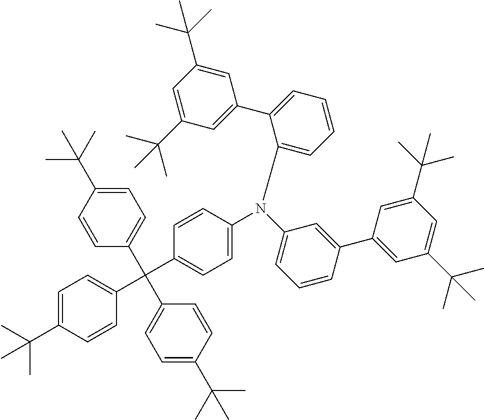 [SMILES: CC(C)(C)c1ccc(C(c2ccc(N(c3cccc(-c4cc(C(C)(C)C)cc(C(C)(C)C)c4)c3)c3ccccc3-c3cc(C(C)(C)C)cc(C(C)(C)C)c3)cc2)(c2ccc(C(C)(C)C)cc2)c2ccc(C(C)(C)C)cc2)cc1] | 15.9 g (77%) | 1031.73 |
| 15 | 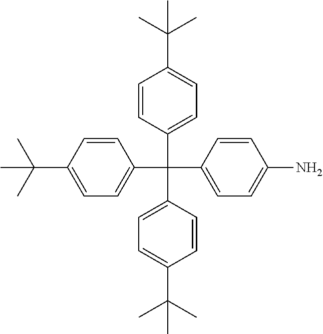 [SMILES: CC(C)(C)c1ccc(C(c2ccc(N)cc2)(c2ccc(C(C)(C)C)cc2)c2ccc(C(C)(C)C)cc2)cc1] | 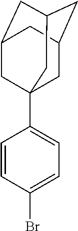 [SMILES: Brc1ccc(C23CC4CC(CC(C4)C2)C3)cc1] | 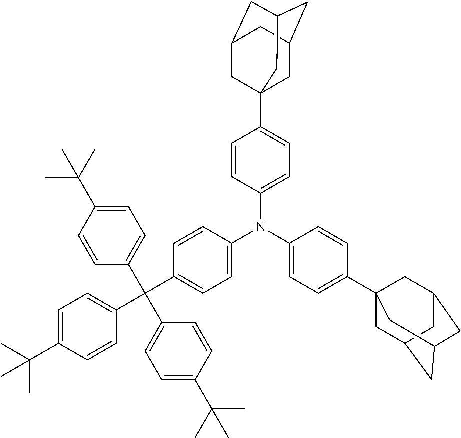 [SMILES: CC(C)(C)c1ccc(C(c2ccc(N(c3ccc(C45CC6CC(CC(C6)C4)C5)cc3)c3ccc(C45CC6CC(CC(C6)C4)C5)cc3)cc2)(c2ccc(C(C)(C)C)cc2)c2ccc(C(C)(C)C)cc2)cc1] | 13.5 g (73%) | 923.64 |
| 16 | 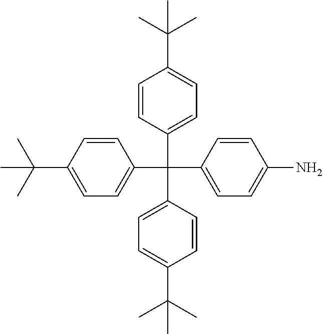 [SMILES: CC(C)(C)c1ccc(C(c2ccc(N)cc2)(c2ccc(C(C)(C)C)cc2)c2ccc(C(C)(C)C)cc2)cc1] | 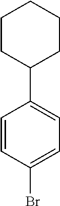 [SMILES: Brc1ccc(C2CCCCC2)cc1] | 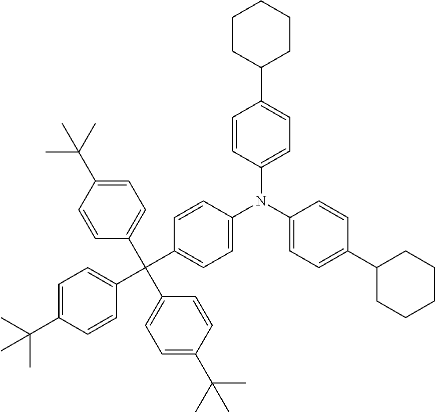 [SMILES: CC(C)(C)c1ccc(C(c2ccc(N(c3ccc(C4CCCCC4)cc3)c3ccc(C4CCCCC4)cc3)cc2)(c2ccc(C(C)(C)C)cc2)c2ccc(C(C)(C)C)cc2)cc1] | 13.3 g (81%) | 819.57 |
| 40 | 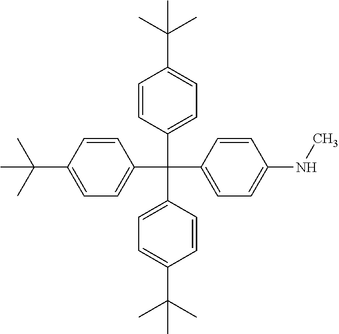 [SMILES: CNc1ccc(C(c2ccc(C(C)(C)C)cc2)(c2ccc(C(C)(C)C)cc2)c2ccc(C(C)(C)C)cc2)cc1] | 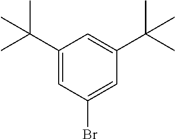 | 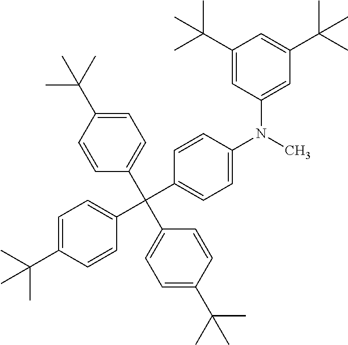 [SMILES: CN(c1ccc(C(c2ccc(C(C)(C)C)cc2)(c2ccc(C(C)(C)C)cc2)c2ccc(C(C)(C)C)cc2)cc1)c1cc(C(C)(C)C)cc(C(C)(C)C)c1] | 11.3 g (80%) | 705.53 |
| 41 | 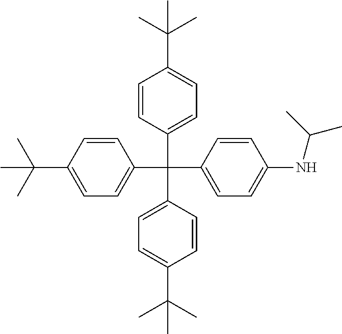 [SMILES: CC(C)Nc1ccc(C(c2ccc(C(C)(C)C)cc2)(c2ccc(C(C)(C)C)cc2)c2ccc(C(C)(C)C)cc2)cc1] | 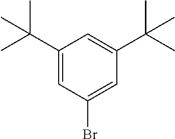 | 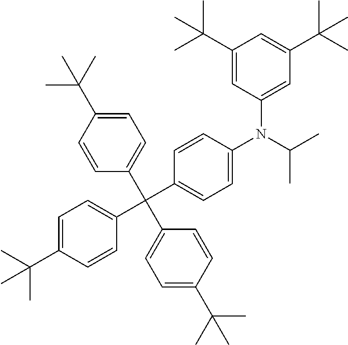 [SMILES: CC(C)N(c1ccc(C(c2ccc(C(C)(C)C)cc2)(c2ccc(C(C)(C)C)cc2)c2ccc(C(C)(C)C)cc2)cc1)c1cc(C(C)(C)C)cc(C(C)(C)C)c1] | 11.5 g (78%) | 733.56 |
| 66 | 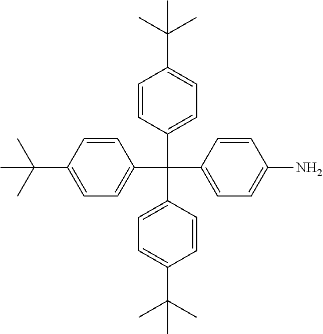 [SMILES: CC(C)(C)c1ccc(C(c2ccc(N)cc2)(c2ccc(C(C)(C)C)cc2)c2ccc(C(C)(C)C)cc2)cc1] | 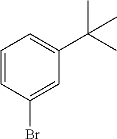 | 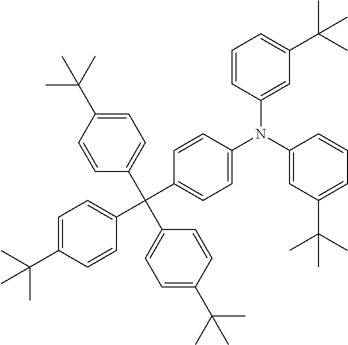 [SMILES: CC(C)(C)c1ccc(C(c2ccc(N(c3cccc(C(C)(C)C)c3)c3cccc(C(C)(C)C)c3)cc2)(c2ccc(C(C)(C)C)cc2)c2ccc(C(C)(C)C)cc2)cc1] | 10.9 g (71%) | 767.54 |
| 111 | 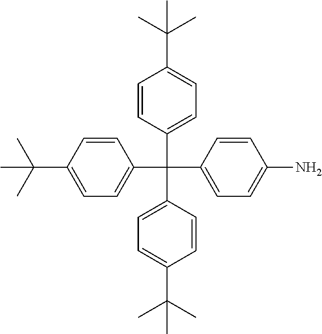 [SMILES: CC(C)(C)c1ccc(C(c2ccc(N)cc2)(c2ccc(C(C)(C)C)cc2)c2ccc(C(C)(C)C)cc2)cc1] | 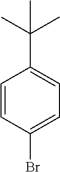 | 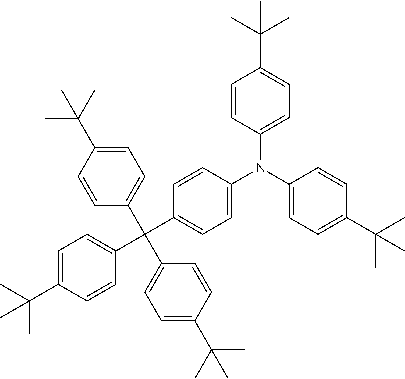 [SMILES: CC(C)(C)c1ccc(N(c2ccc(C(C)(C)C)cc2)c2ccc(C(c3ccc(C(C)(C)C)cc3)(c3ccc(C(C)(C)C)cc3)c3ccc(C(C)(C)C)cc3)cc2)cc1] | 13.5 g (88%) | 767.54 |
| 127 | 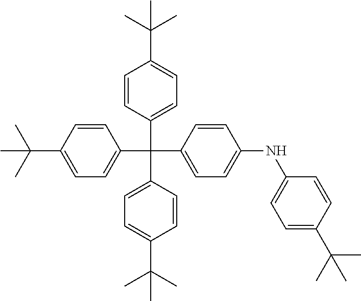 [SMILES: CC(C)(C)c1ccc(Nc2ccc(C(c3ccc(C(C)(C)C)cc3)(c3ccc(C(C)(C)C)cc3)c3ccc(C(C)(C)C)cc3)cc2)cc1] | 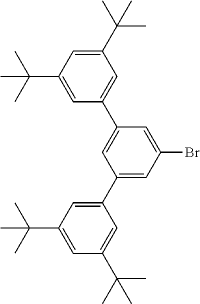 [SMILES: CC(C)(C)c1cc(-c2cc(Br)cc(-c3cc(C(C)(C)C)cc(C(C)(C)C)c3)c2)cc(C(C)(C)C)c1] | 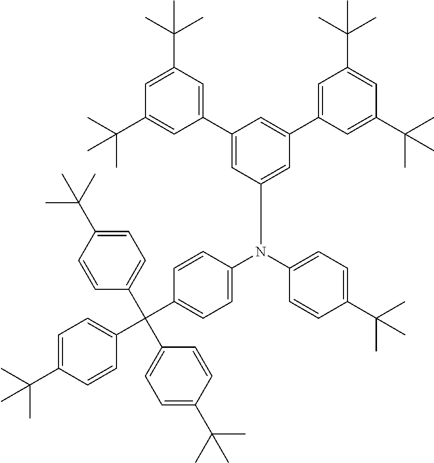 [SMILES: CC(C)(C)c1ccc(N(c2ccc(C(c3ccc(C(C)(C)C)cc3)(c3ccc(C(C)(C)C)cc3)c3ccc(C(C)(C)C)cc3)cc2)c2cc(-c3cc(C(C)(C)C)cc(C(C)(C)C)c3)cc(-c3cc(C(C)(C)C)cc(C(C)(C)C)c3)c2)cc1] | 15.4 g (71%) | 1087.79 |
| 255 | 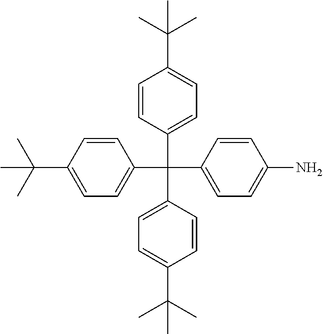 [SMILES: CC(C)(C)c1ccc(C(c2ccc(N)cc2)(c2ccc(C(C)(C)C)cc2)c2ccc(C(C)(C)C)cc2)cc1] | 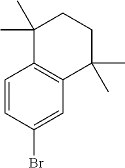 | 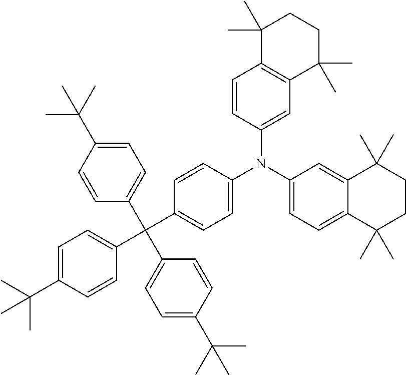 [SMILES: CC(C)(C)c1ccc(C(c2ccc(N(c3ccc4c(c3)C(C)(C)CCC4(C)C)c3ccc4c(c3)C(C)(C)CCC4(C)C)cc2)(c2ccc(C(C)(C)C)cc2)c2ccc(C(C)(C)C)cc2)cc1] | 12.3 g (70%) | 875.64 |
| 267 | 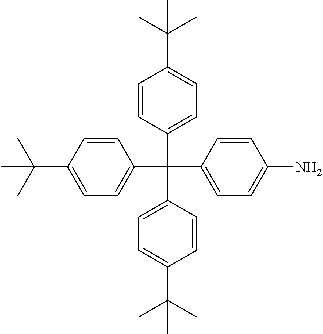 [SMILES: CC(C)(C)c1ccc(C(c2ccc(N)cc2)(c2ccc(C(C)(C)C)cc2)c2ccc(C(C)(C)C)cc2)cc1] | 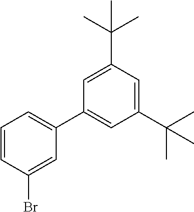 [SMILES: CC(C)(C)c1cc(-c2cccc(Br)c2)cc(C(C)(C)C)c1] | 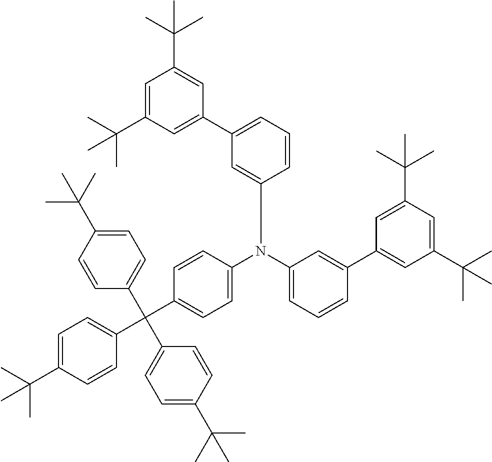 [SMILES: CC(C)(C)c1ccc(C(c2ccc(N(c3cccc(-c4cc(C(C)(C)C)cc(C(C)(C)C)c4)c3)c3cccc(-c4cc(C(C)(C)C)cc(C(C)(C)C)c4)c3)cc2)(c2ccc(C(C)(C)C)cc2)c2ccc(C(C)(C)C)cc2)cc1] | 14.2 g (69%) | 1031.73 |
| 291 | 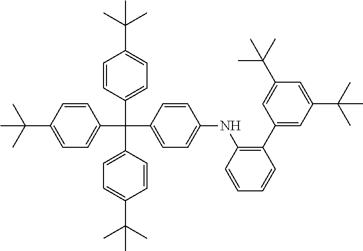 [SMILES: CC(C)(C)c1ccc(C(c2ccc(Nc3ccccc3-c3cc(C(C)(C)C)cc(C(C)(C)C)c3)cc2)(c2ccc(C(C)(C)C)cc2)c2ccc(C(C)(C)C)cc2)cc1] | 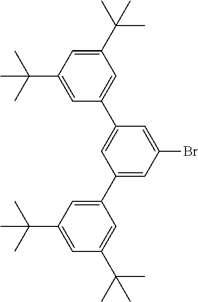 [SMILES: CC(C)(C)c1cc(-c2cc(Br)cc(-c3cc(C(C)(C)C)cc(C(C)(C)C)c3)c2)cc(C(C)(C)C)c1] | 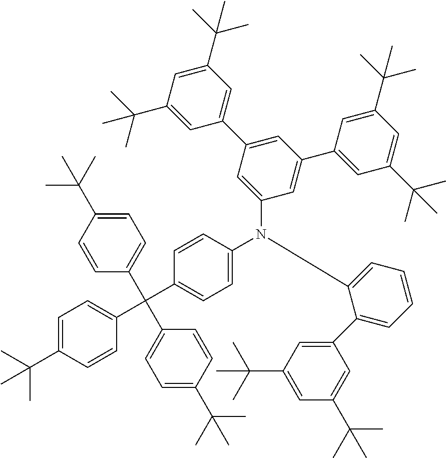 [SMILES: CC(C)(C)c1ccc(C(c2ccc(N(c3cc(-c4cc(C(C)(C)C)cc(C(C)(C)C)c4)cc(-c4cc(C(C)(C)C)cc(C(C)(C)C)c4)c3)c3ccccc3-c3cc(C(C)(C)C)cc(C(C)(C)C)c3)cc2)(c2ccc(C(C)(C)C)cc2)c2ccc(C(C)(C)C)cc2)cc1] | 18.5 g (76%) | 1219.89 |
| 296 | 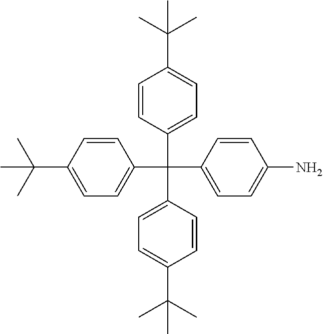 [SMILES: CC(C)(C)c1ccc(C(c2ccc(N)cc2)(c2ccc(C(C)(C)C)cc2)c2ccc(C(C)(C)C)cc2)cc1] | 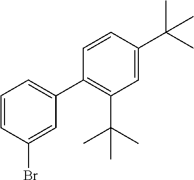 [SMILES: CC(C)(C)c1ccc(-c2cccc(Br)c2)c(C(C)(C)C)c1] | 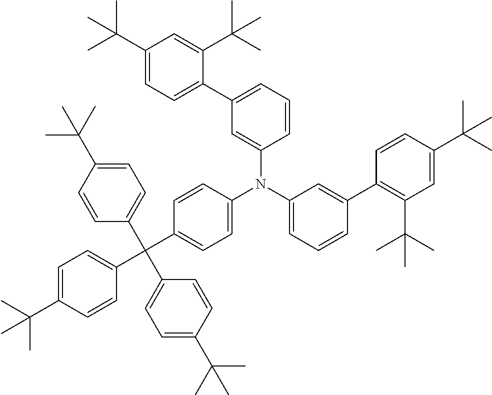 [SMILES: CC(C)(C)c1ccc(C(c2ccc(N(c3cccc(-c4ccc(C(C)(C)C)cc4C(C)(C)C)c3)c3cccc(-c4ccc(C(C)(C)C)cc4C(C)(C)C)c3)cc2)(c2ccc(C(C)(C)C)cc2)c2ccc(C(C)(C)C)cc2)cc1] | 15.5 g (75%) | 1031.73 |
| 304 | 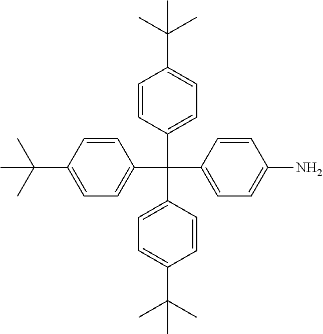 [SMILES: CC(C)(C)c1ccc(C(c2ccc(N)cc2)(c2ccc(C(C)(C)C)cc2)c2ccc(C(C)(C)C)cc2)cc1] | 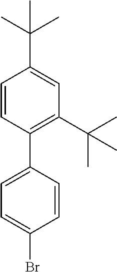 [SMILES: CC(C)(C)c1ccc(-c2ccc(Br)cc2)c(C(C)(C)C)c1] | 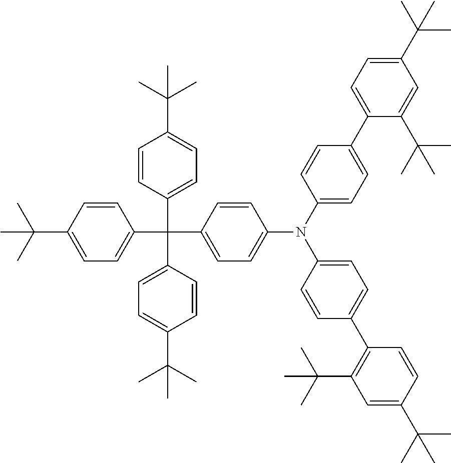 [SMILES: CC(C)(C)c1ccc(C(c2ccc(N(c3ccc(-c4ccc(C(C)(C)C)cc4C(C)(C)C)cc3)c3ccc(-c4ccc(C(C)(C)C)cc4C(C)(C)C)cc3)cc2)(c2ccc(C(C)(C)C)cc2)c2ccc(C(C)(C)C)cc2)cc1] | 14.4 g (70%) | 1031.73 |
| 317 | 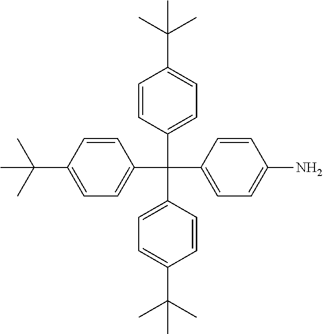 [SMILES: CC(C)(C)c1ccc(C(c2ccc(N)cc2)(c2ccc(C(C)(C)C)cc2)c2ccc(C(C)(C)C)cc2)cc1] | 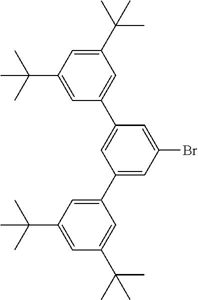 [SMILES: CC(C)(C)c1cc(-c2cc(Br)cc(-c3cc(C(C)(C)C)cc(C(C)(C)C)c3)c2)cc(C(C)(C)C)c1] | 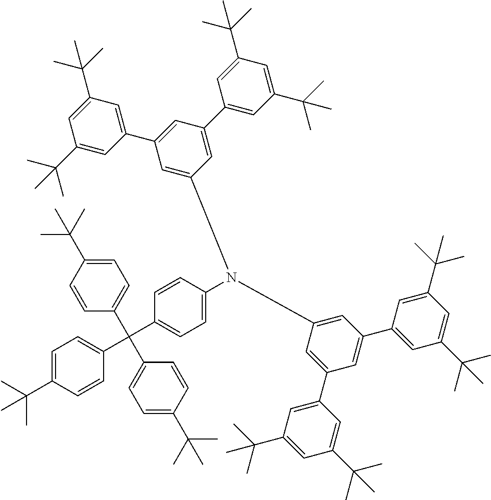 [SMILES: CC(C)(C)c1ccc(C(c2ccc(N(c3cc(-c4cc(C(C)(C)C)cc(C(C)(C)C)c4)cc(-c4cc(C(C)(C)C)cc(C(C)(C)C)c4)c3)c3cc(-c4cc(C(C)(C)C)cc(C(C)(C)C)c4)cc(-c4cc(C(C)(C)C)cc(C(C)(C)C)c4)c3)cc2)(c2ccc(C(C)(C)C)cc2)c2ccc(C(C)(C)C)cc2)cc1] | 24.8 g (88%) | 1408.04 |
| 338 | 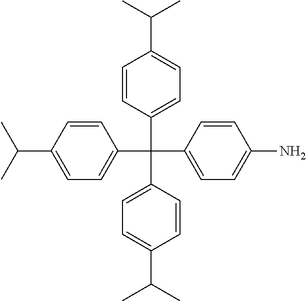 [SMILES: CC(C)c1ccc(C(c2ccc(N)cc2)(c2ccc(C(C)C)cc2)c2ccc(C(C)C)cc2)cc1] | 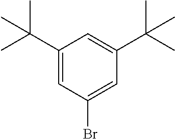 | 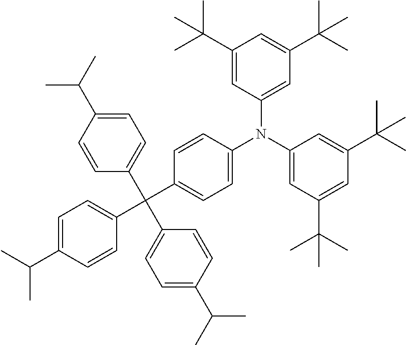 [SMILES: CC(C)c1ccc(C(c2ccc(C(C)C)cc2)(c2ccc(C(C)C)cc2)c2ccc(N(c3cc(C(C)(C)C)cc(C(C)(C)C)c3)c3cc(C(C)(C)C)cc(C(C)(C)C)c3)cc2)cc1] | 13.7 g (82%) | 837.62 |
| 387 | 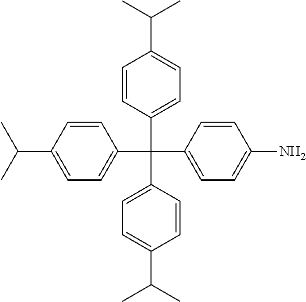 [SMILES: CC(C)c1ccc(C(c2ccc(N)cc2)(c2ccc(C(C)C)cc2)c2ccc(C(C)C)cc2)cc1] | 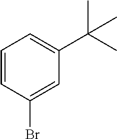 | 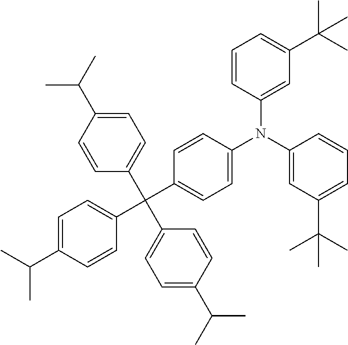 [SMILES: CC(C)c1ccc(C(c2ccc(C(C)C)cc2)(c2ccc(C(C)C)cc2)c2ccc(N(c3cccc(C(C)(C)C)c3)c3cccc(C(C)(C)C)c3)cc2)cc1] | 11.8 g (81%) | 725.50 |
| 432 | 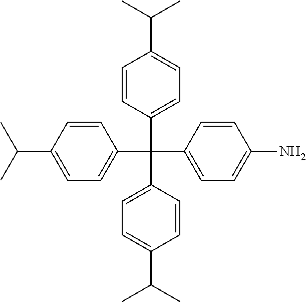 [SMILES: CC(C)c1ccc(C(c2ccc(N)cc2)(c2ccc(C(C)C)cc2)c2ccc(C(C)C)cc2)cc1] | 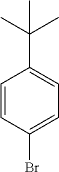 | 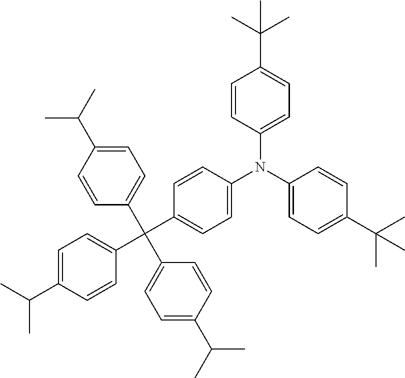 [SMILES: CC(C)c1ccc(C(c2ccc(C(C)C)cc2)(c2ccc(C(C)C)cc2)c2ccc(N(c3ccc(C(C)(C)C)cc3)c3ccc(C(C)(C)C)cc3)cc2)cc1] | 11.6 g (80%) | 725.50 |
| 448 | 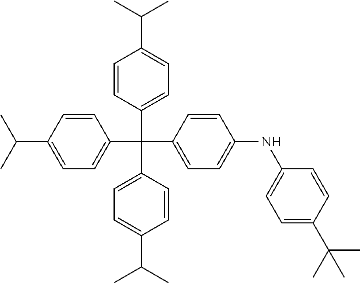 [SMILES: CC(C)c1ccc(C(c2ccc(Nc3ccc(C(C)(C)C)cc3)cc2)(c2ccc(C(C)C)cc2)c2ccc(C(C)C)cc2)cc1] | 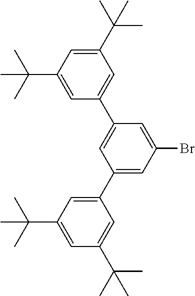 [SMILES: CC(C)(C)c1cc(-c2cc(Br)cc(-c3cc(C(C)(C)C)cc(C(C)(C)C)c3)c2)cc(C(C)(C)C)c1] | 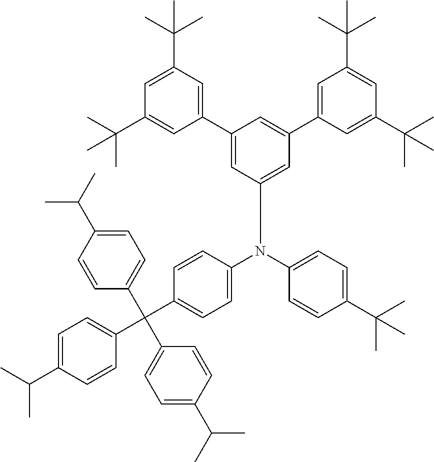 [SMILES: CC(C)c1ccc(C(c2ccc(C(C)C)cc2)(c2ccc(C(C)C)cc2)c2ccc(N(c3ccc(C(C)(C)C)cc3)c3cc(-c4cc(C(C)(C)C)cc(C(C)(C)C)c4)cc(-c4cc(C(C)(C)C)cc(C(C)(C)C)c4)c3)cc2)cc1] | 15.9 g (76%) | 1045.75 |
| 492 | 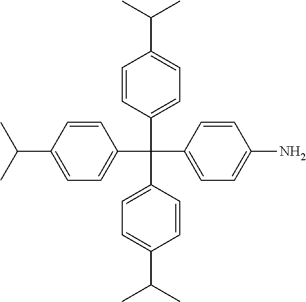 [SMILES: CC(C)c1ccc(C(c2ccc(N)cc2)(c2ccc(C(C)C)cc2)c2ccc(C(C)C)cc2)cc1] | 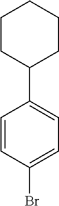 [SMILES: Brc1ccc(C2CCCCC2)cc1] | 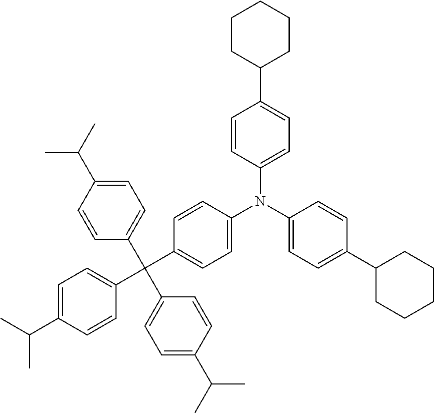 [SMILES: CC(C)c1ccc(C(c2ccc(C(C)C)cc2)(c2ccc(C(C)C)cc2)c2ccc(N(c3ccc(C4CCCCC4)cc3)c3ccc(C4CCCCC4)cc3)cc2)cc1] | 12.8 g (82%) | 777.53 |
| 543 | 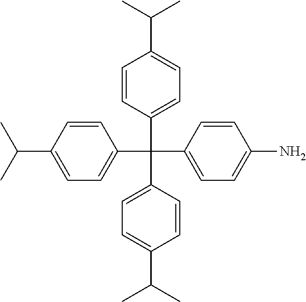 [SMILES: CC(C)c1ccc(C(c2ccc(N)cc2)(c2ccc(C(C)C)cc2)c2ccc(C(C)C)cc2)cc1] | 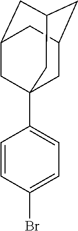 [SMILES: Brc1ccc(C23CC4CC(CC(C4)C2)C3)cc1] | 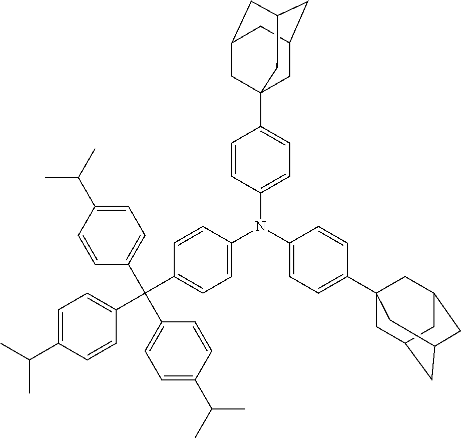 [SMILES: CC(C)c1ccc(C(c2ccc(C(C)C)cc2)(c2ccc(C(C)C)cc2)c2ccc(N(c3ccc(C45CC6CC(CC(C6)C4)C5)cc3)c3ccc(C45CC6CC(CC(C6)C4)C5)cc3)cc2)cc1] | 14.5 g (82%) | 881.59 |
| 585 | 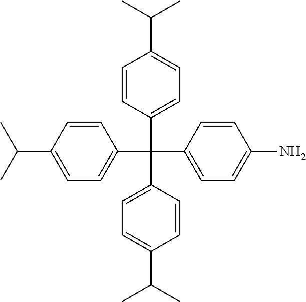 [SMILES: CC(C)c1ccc(C(c2ccc(N)cc2)(c2ccc(C(C)C)cc2)c2ccc(C(C)C)cc2)cc1] | 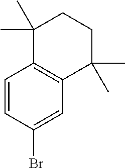 | 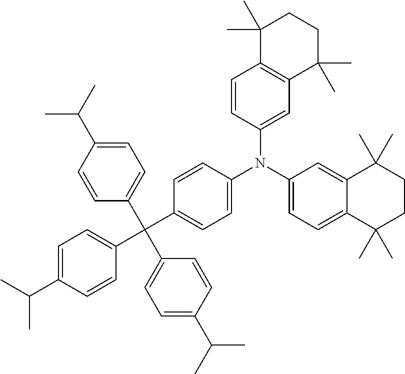 [SMILES: CC(C)c1ccc(C(c2ccc(C(C)C)cc2)(c2ccc(C(C)C)cc2)c2ccc(N(c3ccc4c(c3)C(C)(C)CCC4(C)C)c3ccc4c(c3)C(C)(C)CCC4(C)C)cc2)cc1] | 12.7 g (76%) | 833.59 |
| 597 | 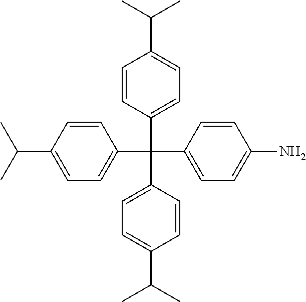 [SMILES: CC(C)c1ccc(C(c2ccc(N)cc2)(c2ccc(C(C)C)cc2)c2ccc(C(C)C)cc2)cc1] | 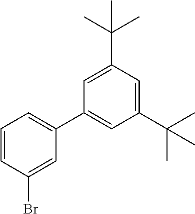 [SMILES: CC(C)(C)c1cc(-c2cccc(Br)c2)cc(C(C)(C)C)c1] | 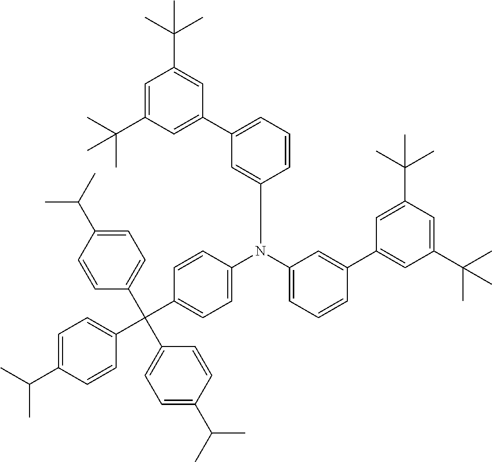 [SMILES: CC(C)c1ccc(C(c2ccc(C(C)C)cc2)(c2ccc(C(C)C)cc2)c2ccc(N(c3cccc(-c4cc(C(C)(C)C)cc(C(C)(C)C)c4)c3)c3cccc(-c4cc(C(C)(C)C)cc(C(C)(C)C)c4)c3)cc2)cc1] | 14.3 g (72%) | 989.68 |
| 599 | 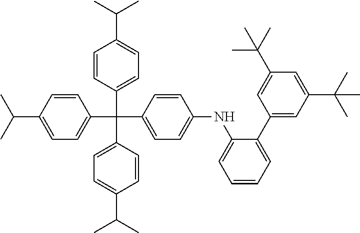 [SMILES: CC(C)c1ccc(C(c2ccc(Nc3ccccc3-c3cc(C(C)(C)C)cc(C(C)(C)C)c3)cc2)(c2ccc(C(C)C)cc2)c2ccc(C(C)C)cc2)cc1] | 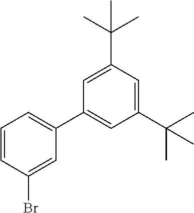 [SMILES: CC(C)(C)c1cc(-c2cccc(Br)c2)cc(C(C)(C)C)c1] | 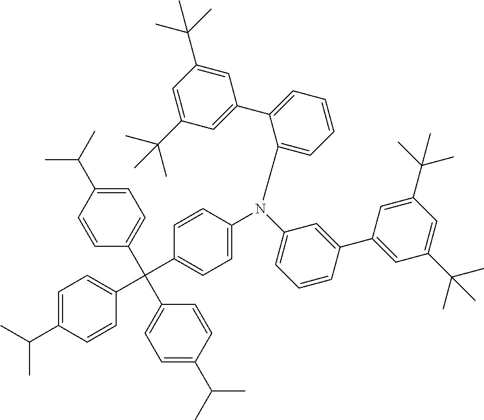 [SMILES: CC(C)c1ccc(C(c2ccc(C(C)C)cc2)(c2ccc(C(C)C)cc2)c2ccc(N(c3cccc(-c4cc(C(C)(C)C)cc(C(C)(C)C)c4)c3)c3ccccc3-c3cc(C(C)(C)C)cc(C(C)(C)C)c3)cc2)cc1] | 14.6 g (74%) | 989.68 |
| 627 | 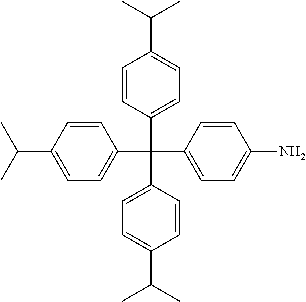 [SMILES: CC(C)c1ccc(C(c2ccc(N)cc2)(c2ccc(C(C)C)cc2)c2ccc(C(C)C)cc2)cc1] | 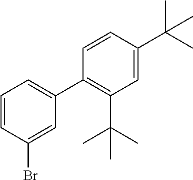 [SMILES: CC(C)(C)c1ccc(-c2cccc(Br)c2)c(C(C)(C)C)c1] | 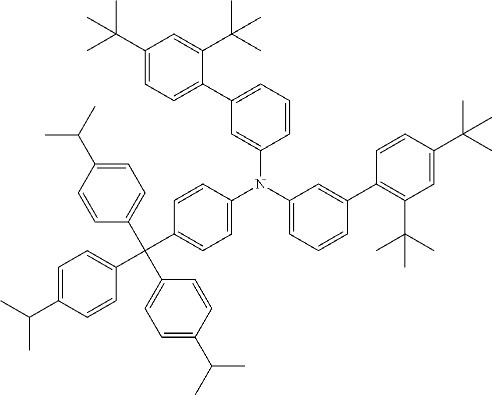 [SMILES: CC(C)c1ccc(C(c2ccc(C(C)C)cc2)(c2ccc(C(C)C)cc2)c2ccc(N(c3cccc(-c4ccc(C(C)(C)C)cc4C(C)(C)C)c3)c3cccc(-c4ccc(C(C)(C)C)cc4C(C)(C)C)c3)cc2)cc1] | 17.4 g (88%) | 989.68 |
| 630 | 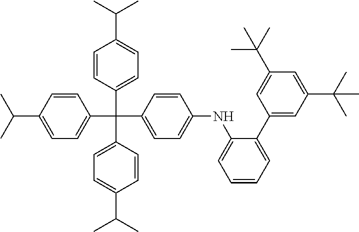 [SMILES: CC(C)c1ccc(C(c2ccc(Nc3ccccc3-c3cc(C(C)(C)C)cc(C(C)(C)C)c3)cc2)(c2ccc(C(C)C)cc2)c2ccc(C(C)C)cc2)cc1] | 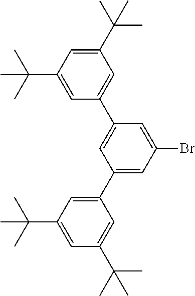 [SMILES: CC(C)(C)c1cc(-c2cc(Br)cc(-c3cc(C(C)(C)C)cc(C(C)(C)C)c3)c2)cc(C(C)(C)C)c1] | 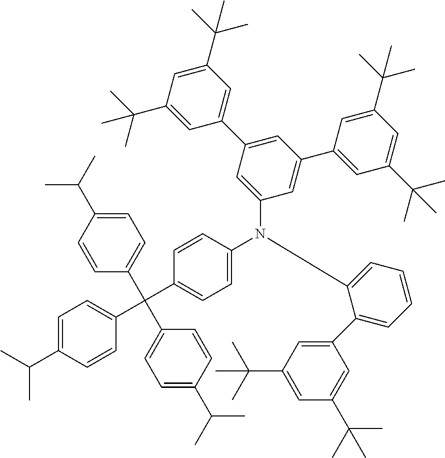 [SMILES: CC(C)c1ccc(C(c2ccc(C(C)C)cc2)(c2ccc(C(C)C)cc2)c2ccc(N(c3cc(-c4cc(C(C)(C)C)cc(C(C)(C)C)c4)cc(-c4cc(C(C)(C)C)cc(C(C)(C)C)c4)c3)c3ccccc3-c3cc(C(C)(C)C)cc(C(C)(C)C)c3)cc2)cc1] | 16.5 g (70%) | 1177.84 |
| 635 | 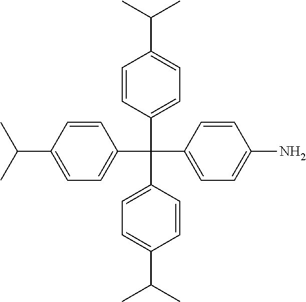 [SMILES: CC(C)c1ccc(C(c2ccc(N)cc2)(c2ccc(C(C)C)cc2)c2ccc(C(C)C)cc2)cc1] | 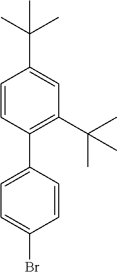 [SMILES: CC(C)(C)c1ccc(-c2ccc(Br)cc2)c(C(C)(C)C)c1] | 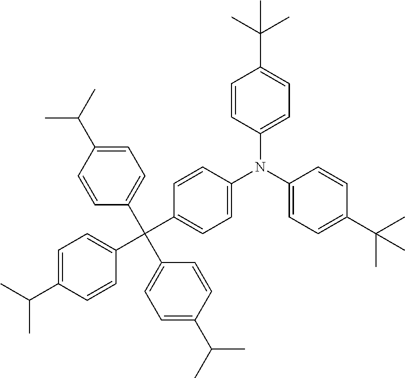 [SMILES: CC(C)c1ccc(C(c2ccc(C(C)C)cc2)(c2ccc(C(C)C)cc2)c2ccc(N(c3ccc(C(C)(C)C)cc3)c3ccc(C(C)(C)C)cc3)cc2)cc1] | 14.3 g (72%) | 989.68 |
| 648 | 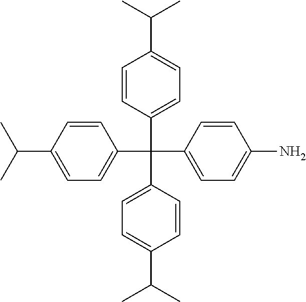 [SMILES: CC(C)c1ccc(C(c2ccc(N)cc2)(c2ccc(C(C)C)cc2)c2ccc(C(C)C)cc2)cc1] | 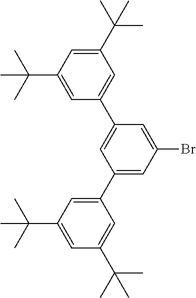 [SMILES: CC(C)(C)c1cc(-c2cc(Br)cc(-c3cc(C(C)(C)C)cc(C(C)(C)C)c3)c2)cc(C(C)(C)C)c1] | 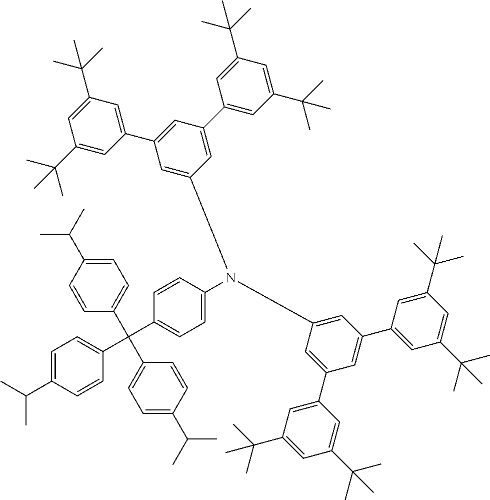 [SMILES: CC(C)c1ccc(C(c2ccc(C(C)C)cc2)(c2ccc(C(C)C)cc2)c2ccc(N(c3cc(-c4cc(C(C)(C)C)cc(C(C)(C)C)c4)cc(-c4cc(C(C)(C)C)cc(C(C)(C)C)c4)c3)c3cc(-c4cc(C(C)(C)C)cc(C(C)(C)C)c4)cc(-c4cc(C(C)(C)C)cc(C(C)(C)C)c4)c3)cc2)cc1] | 19.9 g (73%) | 1366.00 |
| 670 | 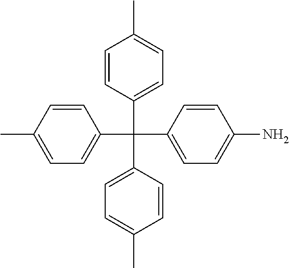 [SMILES: Cc1ccc(C(c2ccc(C)cc2)(c2ccc(C)cc2)c2ccc(N)cc2)cc1] | 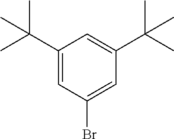 | 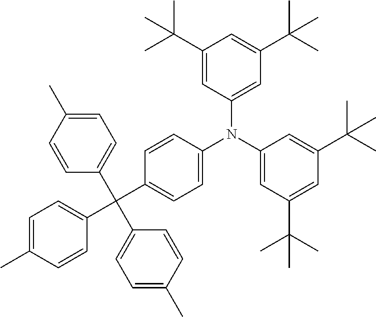 [SMILES: Cc1ccc(C(c2ccc(C)cc2)(c2ccc(C)cc2)c2ccc(N(c3cc(C(C)(C)C)cc(C(C)(C)C)c3)c3cc(C(C)(C)C)cc(C(C)(C)C)c3)cc2)cc1] | 12.1 g (80%) | 753.53 |
| 719 | 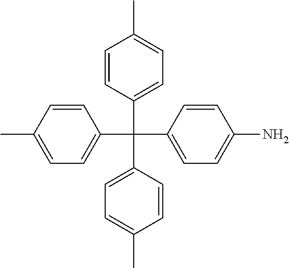 [SMILES: Cc1ccc(C(c2ccc(C)cc2)(c2ccc(C)cc2)c2ccc(N)cc2)cc1] | 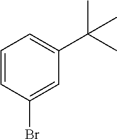 | 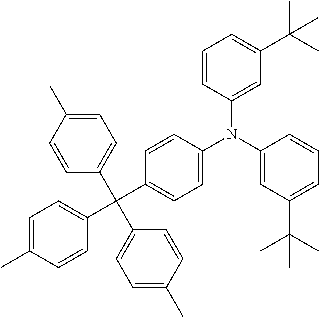 [SMILES: Cc1ccc(C(c2ccc(C)cc2)(c2ccc(C)cc2)c2ccc(N(c3cccc(C(C)(C)C)c3)c3cccc(C(C)(C)C)c3)cc2)cc1] | 10.0 g (78%) | 641.40 |
| 764 | 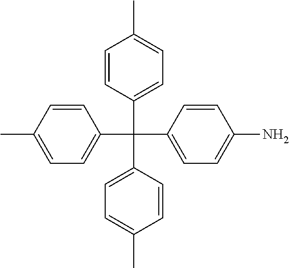 [SMILES: Cc1ccc(C(c2ccc(C)cc2)(c2ccc(C)cc2)c2ccc(N)cc2)cc1] | 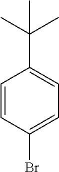 | 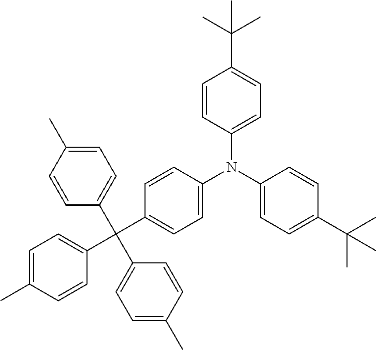 [SMILES: Cc1ccc(C(c2ccc(C)cc2)(c2ccc(C)cc2)c2ccc(N(c3ccc(C(C)(C)C)cc3)c3ccc(C(C)(C)C)cc3)cc2)cc1] | 11.4 g (89%) | 641.40 |
| 780 | 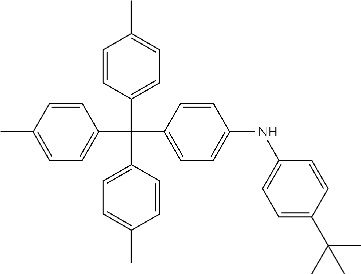 [SMILES: Cc1ccc(C(c2ccc(C)cc2)(c2ccc(C)cc2)c2ccc(Nc3ccc(C(C)(C)C)cc3)cc2)cc1] | 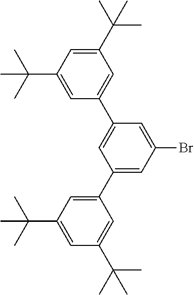 [SMILES: CC(C)(C)c1cc(-c2cc(Br)cc(-c3cc(C(C)(C)C)cc(C(C)(C)C)c3)c2)cc(C(C)(C)C)c1] | 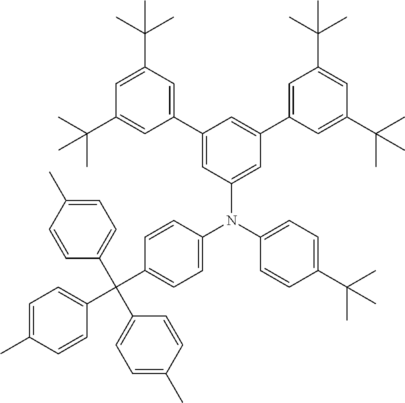 [SMILES: Cc1ccc(C(c2ccc(C)cc2)(c2ccc(C)cc2)c2ccc(N(c3ccc(C(C)(C)C)cc3)c3cc(-c4cc(C(C)(C)C)cc(C(C)(C)C)c4)cc(-c4cc(C(C)(C)C)cc(C(C)(C)C)c4)c3)cc2)cc1] | 11.7 g (82%) | 961.65 |
| 824 | 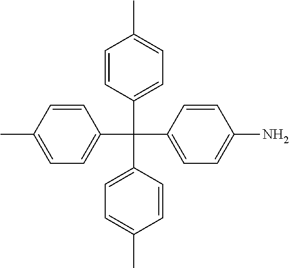 [SMILES: Cc1ccc(C(c2ccc(C)cc2)(c2ccc(C)cc2)c2ccc(N)cc2)cc1] | 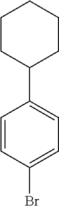 [SMILES: Brc1ccc(C2CCCCC2)cc1] | 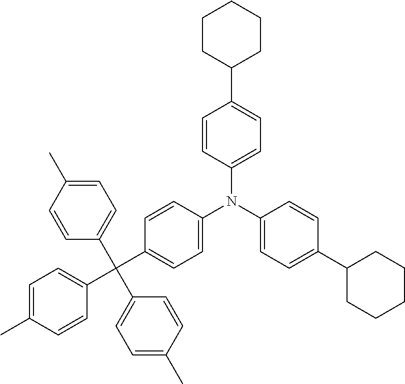 [SMILES: Cc1ccc(C(c2ccc(C)cc2)(c2ccc(C)cc2)c2ccc(N(c3ccc(C4CCCCC4)cc3)c3ccc(C4CCCCC4)cc3)cc2)cc1] | 12.2 g (88%) | 693.43 |
| 875 | 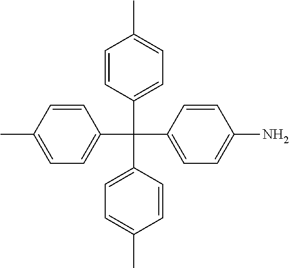 [SMILES: Cc1ccc(C(c2ccc(C)cc2)(c2ccc(C)cc2)c2ccc(N)cc2)cc1] | 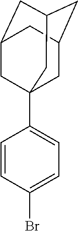 [SMILES: Brc1ccc(C23CC4CC(CC(C4)C2)C3)cc1] | 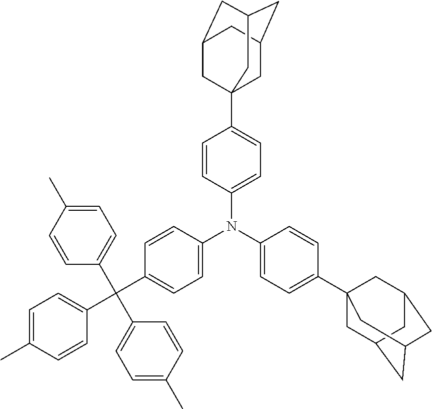 [SMILES: Cc1ccc(C(c2ccc(C)cc2)(c2ccc(C)cc2)c2ccc(N(c3ccc(C45CC6CC(CC(C6)C4)C5)cc3)c3ccc(C45CC6CC(CC(C6)C4)C5)cc3)cc2)cc1] | 13.6 g (85%) | 797.50 |
| 917 | 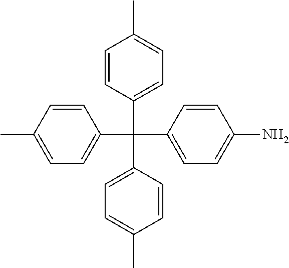 [SMILES: Cc1ccc(C(c2ccc(C)cc2)(c2ccc(C)cc2)c2ccc(N)cc2)cc1] | 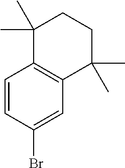 | 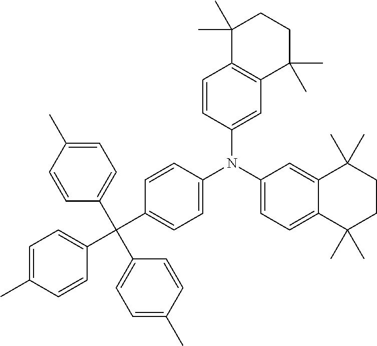 [SMILES: Cc1ccc(C(c2ccc(C)cc2)(c2ccc(C)cc2)c2ccc(N(c3ccc4c(c3)C(C)(C)CCC4(C)C)c3ccc4c(c3)C(C)(C)CCC4(C)C)cc2)cc1] | 12.0 g (80%) | 749.50 |
| 929 | 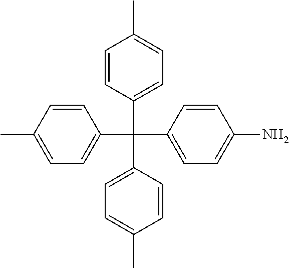 [SMILES: Cc1ccc(C(c2ccc(C)cc2)(c2ccc(C)cc2)c2ccc(N)cc2)cc1] | 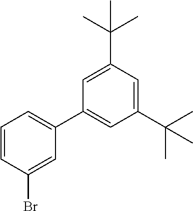 [SMILES: CC(C)(C)c1cc(-c2cccc(Br)c2)cc(C(C)(C)C)c1] | 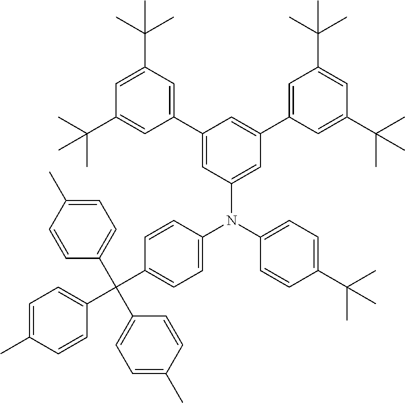 [SMILES: Cc1ccc(C(c2ccc(C)cc2)(c2ccc(C)cc2)c2ccc(N(c3ccc(C(C)(C)C)cc3)c3cc(-c4cc(C(C)(C)C)cc(C(C)(C)C)c4)cc(-c4cc(C(C)(C)C)cc(C(C)(C)C)c4)c3)cc2)cc1] | 15.6 g (86%) | 905.59 |
| 931 | 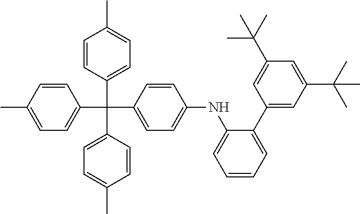 [SMILES: Cc1ccc(C(c2ccc(C)cc2)(c2ccc(C)cc2)c2ccc(Nc3ccccc3-c3cc(C(C)(C)C)cc(C(C)(C)C)c3)cc2)cc1] | 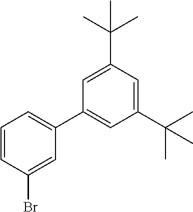 [SMILES: CC(C)(C)c1cc(-c2cccc(Br)c2)cc(C(C)(C)C)c1] | 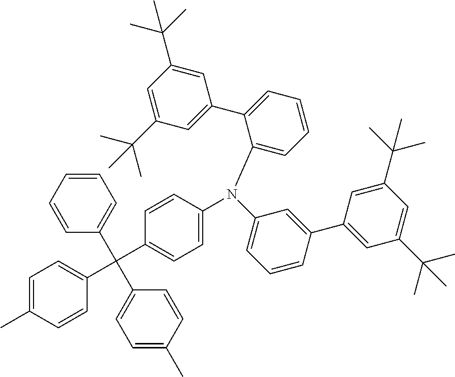 [SMILES: Cc1ccc(C(c2ccccc2)(c2ccc(C)cc2)c2ccc(N(c3cccc(-c4cc(C(C)(C)C)cc(C(C)(C)C)c4)c3)c3ccccc3-c3cc(C(C)(C)C)cc(C(C)(C)C)c3)cc2)cc1] | 12.5 g (88%) | 905.56 |
| 959 | 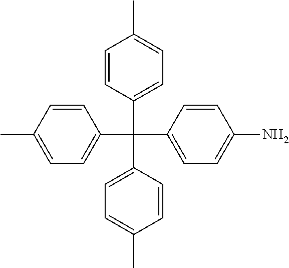 [SMILES: Cc1ccc(C(c2ccc(C)cc2)(c2ccc(C)cc2)c2ccc(N)cc2)cc1] | 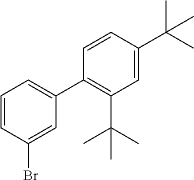 [SMILES: CC(C)(C)c1ccc(-c2cccc(Br)c2)c(C(C)(C)C)c1] | 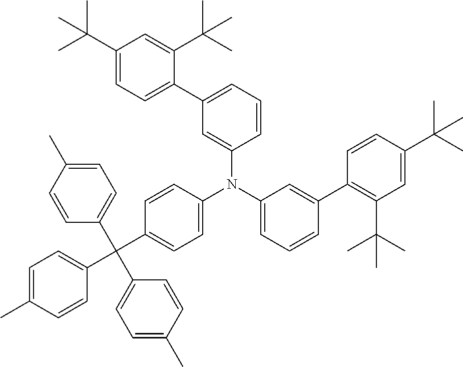 [SMILES: Cc1ccc(C(c2ccc(C)cc2)(c2ccc(C)cc2)c2ccc(N(c3cccc(-c4ccc(C(C)(C)C)cc4C(C)(C)C)c3)c3cccc(-c4ccc(C(C)(C)C)cc4C(C)(C)C)c3)cc2)cc1] | 16.1 g (89%) | 905.59 |
| 962 | 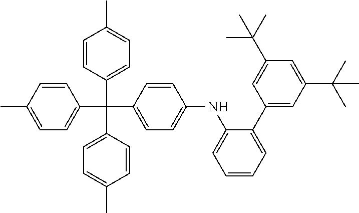 [SMILES: Cc1ccc(C(c2ccc(C)cc2)(c2ccc(C)cc2)c2ccc(Nc3ccccc3-c3cc(C(C)(C)C)cc(C(C)(C)C)c3)cc2)cc1] | 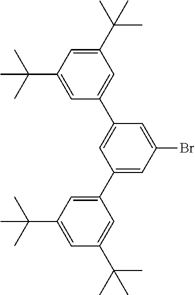 [SMILES: CC(C)(C)c1cc(-c2cc(Br)cc(-c3cc(C(C)(C)C)cc(C(C)(C)C)c3)c2)cc(C(C)(C)C)c1] | 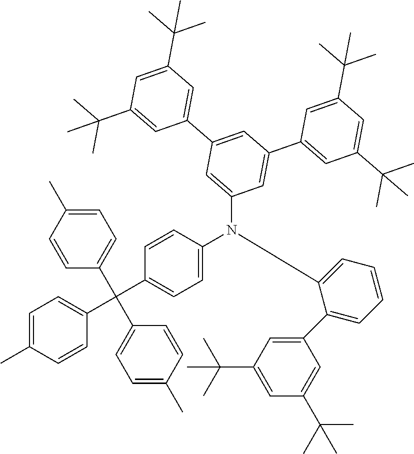 [SMILES: Cc1ccc(C(c2ccc(C)cc2)(c2ccc(C)cc2)c2ccc(N(c3cc(-c4cc(C(C)(C)C)cc(C(C)(C)C)c4)cc(-c4cc(C(C)(C)C)cc(C(C)(C)C)c4)c3)c3ccccc3-c3cc(C(C)(C)C)cc(C(C)(C)C)c3)cc2)cc1] | 12.4 g (87%) | 1093.75 |
| 967 | 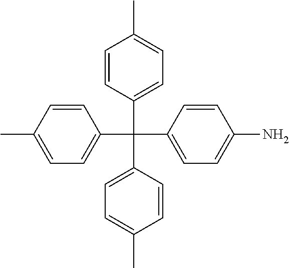 [SMILES: Cc1ccc(C(c2ccc(C)cc2)(c2ccc(C)cc2)c2ccc(N)cc2)cc1] | 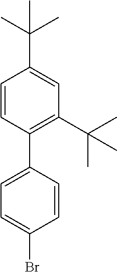 [SMILES: CC(C)(C)c1ccc(-c2ccc(Br)cc2)c(C(C)(C)C)c1] | 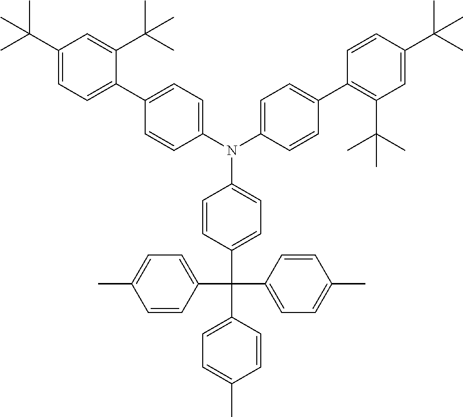 [SMILES: Cc1ccc(C(c2ccc(C)cc2)(c2ccc(C)cc2)c2ccc(N(c3ccc(-c4ccc(C(C)(C)C)cc4C(C)(C)C)cc3)c3ccc(-c4ccc(C(C)(C)C)cc4C(C)(C)C)cc3)cc2)cc1] | 14.5 g (80%) | 905.59 |
| 980 | 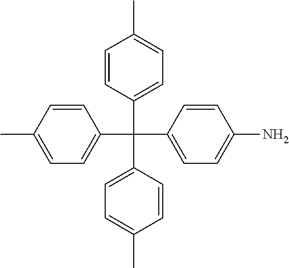 [SMILES: Cc1ccc(C(c2ccc(C)cc2)(c2ccc(C)cc2)c2ccc(N)cc2)cc1] | 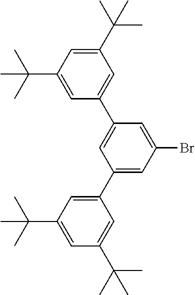 [SMILES: CC(C)(C)c1cc(-c2cc(Br)cc(-c3cc(C(C)(C)C)cc(C(C)(C)C)c3)c2)cc(C(C)(C)C)c1] | 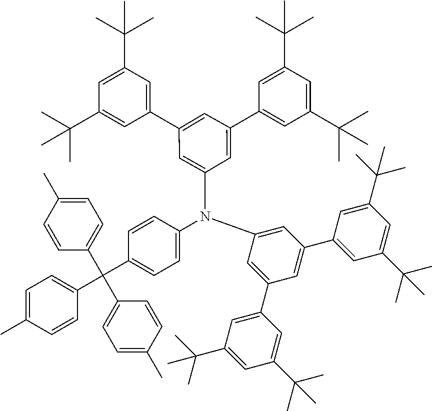 [SMILES: Cc1ccc(C(c2ccc(C)cc2)(c2ccc(C)cc2)c2ccc(N(c3cc(-c4cc(C(C)(C)C)cc(C(C)(C)C)c4)cc(-c4cc(C(C)(C)C)cc(C(C)(C)C)c4)c3)c3cc(-c4cc(C(C)(C)C)cc(C(C)(C)C)c4)cc(-c4cc(C(C)(C)C)cc(C(C)(C)C)c4)c3)cc2)cc1] | 20.0 g (78%) | 1281.90 |

### EXPERIMENTAL EXAMPLES

The compound of the present disclosure was confirmed to have an effect through the following experiments, which are only typical examples, and the experimental examples are not limited thereto.

As a typical example, an experiment to confirm the single film properties (refractive index and transmittance) of Compound 1 is described, and the compounds represented by Chemical Formula 1 of the present disclosure include the same structure as Compound 1 and may have a similar degree of effect.

### Experimental Example 1—Confirmation of Single Film Properties (Refractive Index and Transmittance)

In order to measure optical properties (refractive index and transmittance), 1,000 Å of each of Compound 1, Compound 338 and Compound 670 among the compounds in Table 14, and Comparative Compounds 1 to 3 below were deposited on a glass substrate (0.7T) at a vacuum degree of 9×10⁻⁷ Torr at a rate of 1 Å/sec to form a single film.

As shown in Table 15 below, the refractive index and transmittance (%) of the single film for evaluating optical properties were measured for each single film-forming material compound using an Ellipsometer from J.A. WOOLLAM.

### Comparative Compounds 1 to 3

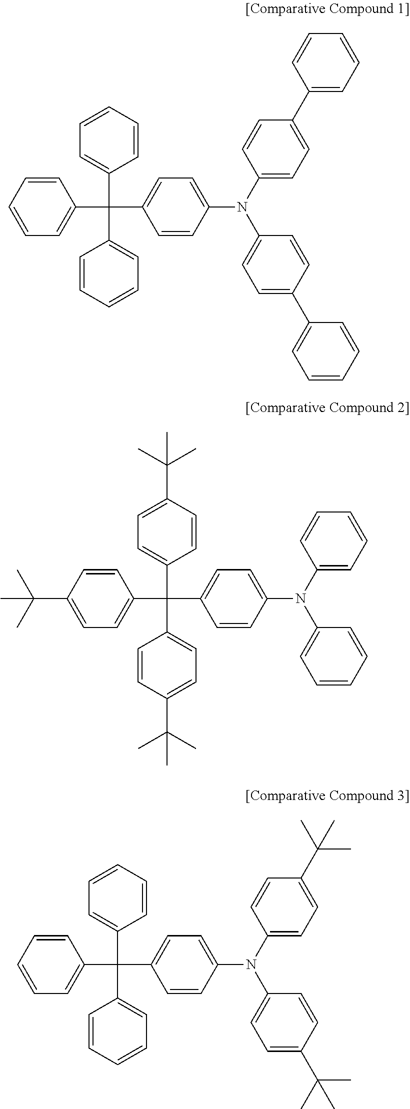 [SMILES: c1ccc(-c2ccc(N(c3ccc(-c4ccccc4)cc3)c3ccc(C(c4ccccc4)(c4ccccc4)c4ccccc4)cc3)cc2)cc1 ; CC(C)(C)c1ccc(C(c2ccc(N(c3ccccc3)c3ccccc3)cc2)(c2ccc(C(C)(C)C)cc2)c2ccc(C(C)(C)C)cc2)cc1 ; CC(C)(C)c1ccc(N(c2ccc(C(C)(C)C)cc2)c2ccc(C(c3ccccc3)(c3ccccc3)c3ccccc3)cc2)cc1]

TABLE 15

| Single film- | 400-410 nm | @460 nm | | @520 nm | | @620 nm | |
|---|---|---|---|---|---|---|---|
| forming | Transmittance | Refractive | Transmittance | Refractive | Transmittance | Refractive | Transmittance |
| material | (%) | index | (%) | index | (%) | index | (%) |
| Compound 1 | 97 | 1.57 | 100 | 1.58 | 100 | 1.59 | 100 |
| Compound | 96 | 1.57 | 100 | 1.57 | 100 | 1.56 | 100 |
| 338 | | | | | | | |
| Compound | 95 | 1.58 | 100 | 1.58 | 100 | 1.56 | 100 |
| 670 | | | | | | | |
| Comparative | 78 | 1.88 | 99 | 1.88 | 100 | 1.89 | 100 |
| Compound 1 | | | | | | | |
| Comparative | 84 | 1.76 | 100 | 1.75 | 100 | 1.75 | 100 |
| Compound 2 | | | | | | | |
| Comparative | 82 | 1.76 | 99 | 1.76 | 100 | 1.77 | 100 |
| Compound 3 | | | | | | | |

Referring to Table 15 above, examining the optical properties, it can be confirmed that Compound 1, Compound 338 and Compound 670 all have low refractive indices of less than 1.6 at wavelengths of 460 nm, 520 nm and 620 nm. On the other hand, Comparative Compounds 1 to 3 all have high refractive indices of 1.75 or higher at wavelengths of 460 nm, 520 nm and 620 nm.

When examining the transmittance of Compound 1, Compound 338 and Compound 670 at wavelengths of 460 nm, 520 nm and 620 nm, it can be confirmed that all have high transmittance of 100%.

In addition, Compound 1, Compound 338 and Compound 670 all have transmittances of 90% or higher at wavelengths of 400 nm or more and 410 nm or less, minimizing the loss of light generated from the diode and realizing high efficiency. On the other hand, for Comparative Compounds 1 to 3, it can be confirmed that the light transmittance is relatively low compared to Compound 1, Compound 338 and Compound 670.

### Experimental Example 2—Confirmation of Diode Properties

In order to confirm the diode properties of the compounds, present examples and comparative examples were made as follows.

### Present Example 1

A substrate on which an ITO anode (100 nm) of an organic light-emitting diode was stacked was patterned, while distinguishing cathode and anode areas and an insulating layer, through a photolithography process, and then, subjected to UV-ozone treatment and surface treatment using O₂:N₂ plasma for increasing the work-function of the anode (ITO) and cleaning.

Then, on the anode, 1,4,5,8,9,11-hexaazatriphenylen-hexacarbonitrile (HAT-CN) was formed into a thickness of 10 nm as a hole injection layer (HIL).

On the hole injection layer, N4,N4,N4′,N4′-tetra([1,1′-biphenyl]-4-yl)-[1,1′-biphenyl]-4,4′-diamine was vacuum deposited into a thickness of 90 nm as a hole transport layer (HTL), and on the hole transport layer, N-phenyl-N-(4-(spiro[benzo[d,e]anthracene-7,9′-fluorene]-2′-yl)phenyl)dibenzo[b,d]furan-4-amine was formed into a thickness of 15 nm as an electron blocking layer (EBL).

On the electron blocking layer (EBL), 9,10-bis(2-naphthyl)anthracene (ADN) was deposited to 25 nm as a host, and about 3 wt % of 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB) was doped as a dopant.

Then, 2-(4-(9,10-di(naphthalen-2-yl)anthracen-2-yl)phenyl)-1-phenyl-1H-benzo[d]imidazole and LiQ were mixed in a weight ratio of 1:1 and deposited into 25 nm thereon as an electron transport layer (ETL), and on the electron transport layer, an electron injection layer (LiQ) was deposited into 1 nm, and aluminum (Al) was deposited into a thickness of 100 nm as a cathode.

On the cathode, a compound N4,N4′-bis(4-(benzo[d]oxazol-2-yl)phenyl)-N4,N4′-diphenyl-[1,1′-biphenyl]-4,4′-diamine was deposited into a thickness of 1,000 Å as a high-refractive capping layer, and then Compound 1 of Synthesis Example 1 was deposited into a thickness of 400 Å as a low-refractive capping layer.

Then, on the capping layer (CPL), a seal cap was attached using a UV curable adhesive so as to protect an organic light-emitting diode from oxygen (O₂) or moisture in the air to manufacture an organic light-emitting diode according to Example 1.

### Present Examples 2 to 41

Organic light-emitting diodes according to Examples 2 to 41 were manufactured by the same manner as Example 1 except for using the compounds of Table 14 instead of Compound 1 in the low-refractive capping layer of Example 1.

### Comparative Examples 1 to 6

Organic light-emitting diodes according to Comparative Examples 1 to 6 were manufactured by the same manner as Example 1 except for using Comparative Compounds 1 to 3 above and Comparative Compounds 4 to 6 below instead of Compound 1 in the low-refractive capping layer of Example 1.

### Comparative Compounds 4 to 6

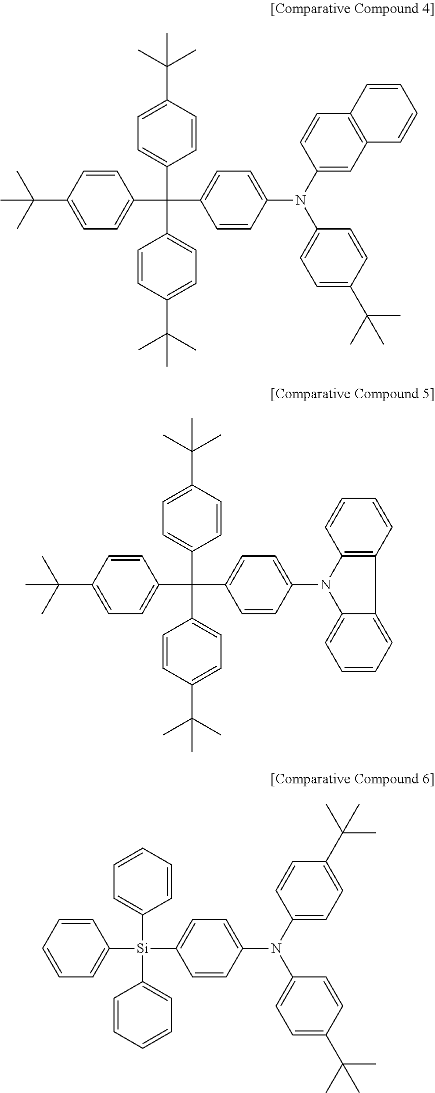 [SMILES: CC(C)(C)c1ccc(N(c2ccc(C(c3ccc(C(C)(C)C)cc3)(c3ccc(C(C)(C)C)cc3)c3ccc(C(C)(C)C)cc3)cc2)c2ccc3ccccc3c2)cc1 ; CC(C)(C)c1ccc(C(c2ccc(-n3c4ccccc4c4ccccc43)cc2)(c2ccc(C(C)(C)C)cc2)c2ccc(C(C)(C)C)cc2)cc1 ; CC(C)(C)c1ccc(N(c2ccc(C(C)(C)C)cc2)c2ccc([Si](c3ccccc3)(c3ccccc3)c3ccccc3)cc2)cc1]

For the organic light-emitting diodes of Examples 1 to 41 and Comparative Examples 1 to 6, the efficiency (Cd/A) was measured by applying a current of 10 mA/cm² with a CS-2000 from KONICA MINOLTA, and the lifetime (LT95) was measured by confirming the time for the luminance to decrease from an initial luminance to 95% with a constant current drive of 10 mA/cm² with a M6000 from McScience.

The measurement results are shown in Table 16 below.

TABLE 16

| Present Example/ | Material for | Efficiency | Lifetime |
|---|---|---|---|
| Comparative Example | capping layer | (Cd/A) | (LT95) |
| Present Example 1 | Compound 1 | 8.73 | 304 |
| Present Example 2 | Compound 66 | 8.70 | 298 |
| Present Example 3 | Compound 111 | 8.67 | 299 |
| Present Example 4 | Compound 127 | 8.72 | 301 |
| Present Example 5 | Compound 16 | 8.70 | 300 |
| Present Example 6 | Compound 15 | 8.73 | 302 |
| Present Example 7 | Compound 255 | 8.66 | 297 |
| Present Example 8 | Compound 267 | 8.59 | 299 |
| Present Example 9 | Compound 5 | 8.60 | 296 |
| Present Example 10 | Compound 291 | 8.58 | 303 |
| Present Example 11 | Compound 296 | 8.59 | 296 |
| Present Example 12 | Compound 304 | 8.60 | 294 |
| Present Example 13 | Compound 317 | 8.55 | 293 |
| Present Example 14 | Compound 338 | 8.77 | 308 |
| Present Example 15 | Compound 387 | 8.69 | 303 |
| Present Example 16 | Compound 432 | 8.71 | 304 |
| Present Example 17 | Compound 448 | 8.69 | 306 |
| Present Example 18 | Compound 492 | 8.72 | 299 |
| Present Example 19 | Compound 543 | 8.75 | 295 |
| Present Example 20 | Compound 585 | 8.74 | 300 |
| Present Example 21 | Compound 597 | 8.69 | 293 |
| Present Example 22 | Compound 599 | 8.66 | 301 |
| Present Example 23 | Compound 627 | 8.63 | 302 |
| Present Example 24 | Compound 630 | 8.70 | 300 |
| Present Example 25 | Compound 635 | 8.65 | 299 |
| Present Example 26 | Compound 648 | 8.63 | 295 |
| Present Example 27 | Compound 670 | 8.71 | 310 |
| Present Example 28 | Compound 719 | 8.59 | 305 |
| Present Example 29 | Compound 764 | 8.66 | 302 |
| Present Example 30 | Compound 780 | 8.61 | 299 |
| Present Example 31 | Compound 824 | 8.59 | 296 |
| Present Example 32 | Compound 875 | 8.65 | 303 |
| Present Example 33 | Compound 917 | 8.63 | 301 |
| Present Example 34 | Compound 929 | 8.60 | 297 |
| Present Example 35 | Compound 931 | 8.59 | 293 |
| Present Example 36 | Compound 959 | 8.57 | 295 |
| Present Example 37 | Compound 962 | 8.56 | 296 |
| Present Example 38 | Compound 967 | 8.59 | 299 |
| Present Example 39 | Compound 980 | 8.55 | 301 |
| Present Example 40 | Compound 40 | 8.52 | 299 |
| Present Example 41 | Compound 41 | 8.61 | 304 |
| Comparative | Comparative | 6.16 | 281 |
| Example 1 | Compound 1 | | |
| Comparative | Comparative | 6.89 | 291 |
| Example 2 | Compound 2 | | |
| Comparative | Comparative | 6.88 | 285 |
| Example 3 | Compound 3 | | |
| Comparative | Comparative | 6.54 | 284 |
| Example 4 | Compound 4 | | |
| Comparative | Comparative | 6.02 | 288 |
| Example 5 | Compound 5 | | |
| Comparative | Comparative | 6.06 | 285 |
| Example 6 | Compound 6 | | |

Referring to Table 16 above, it can be confirmed that the efficiency of the organic light-emitting diodes to which the compounds according to the Examples are applied is greater than about 8.5 Cd/A, while the efficiency of the organic light-emitting diodes to which the compounds according to the Comparative Examples are applied is less than about 7 Cd/A, confirming that the efficiency of the organic light-emitting diodes according to the Present Examples is significantly superior to the efficiency of the organic light-emitting diodes according to the Comparative Examples.

In addition, in the case of the lifetime, it can be confirmed that the lifetime of the organic light-emitting diodes according to the Present Examples is relatively longer than the lifetime of the organic light-emitting diodes according to the Comparative Examples.

### Experimental Examples 3—Confirmation of Diode Properties

The shape of the low-refractive capping layer of the organic light-emitting diode according to Example 1 was observed through a JEM-ARM200F model scanning electron microscope (SEM) of JEOL, and the results are shown in FIG. 1.

Referring to FIG. 1, it can be confirmed that the thin film arrangement of the molecules of the low-refractive capping layer to which the compounds of the present disclosure are applied is excellent, and it can be confirmed that an amorphous thin film may be formed, thereby forming a transparent and smooth cross-section.

In addition, referring to FIG. 2, it can be confirmed that a transparent thin film is formed in the deposition process.

Through the properties, it can be confirmed that the organic light-emitting diode including the capping layer to which the compound according to the Present Example is applied has high efficiency as shown in Table 16 above.

Although embodiments of the present disclosure have been described in detail above, the scope of the present disclosure is not limited to the above embodiments, but various modifications and improvements made by those skilled in the art using the basic concept of the present disclosure defined in the following claims also fall within the scope of the present disclosure.

Additional Examples of Organic Compound (fluorine substitution) of the invention.

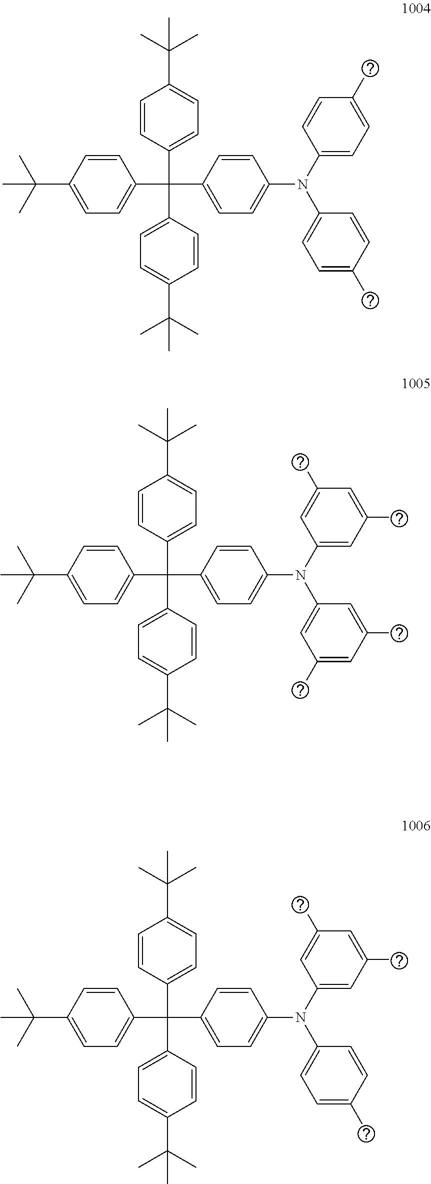

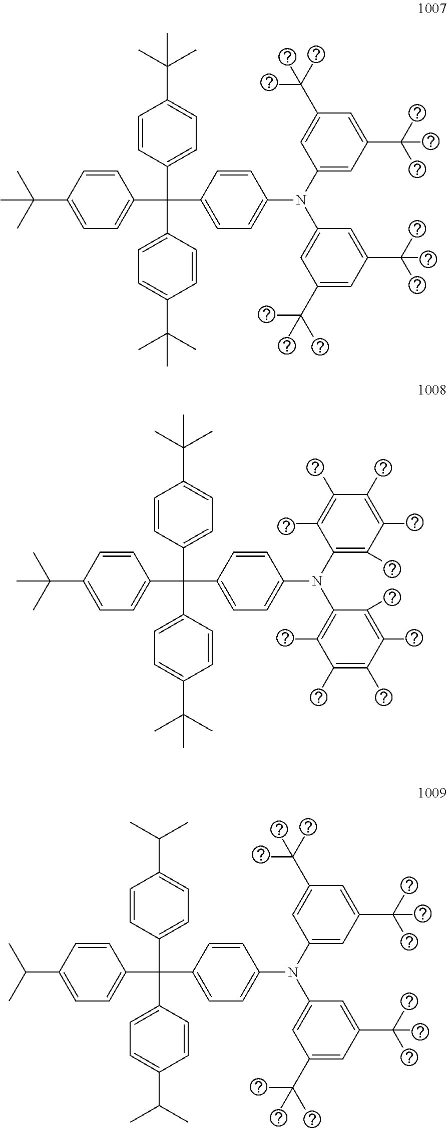

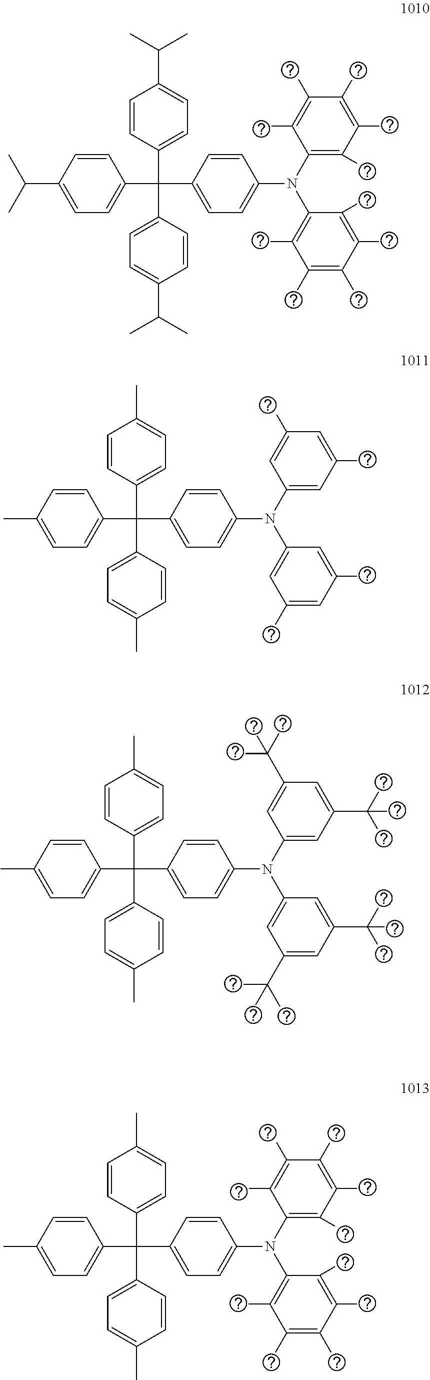

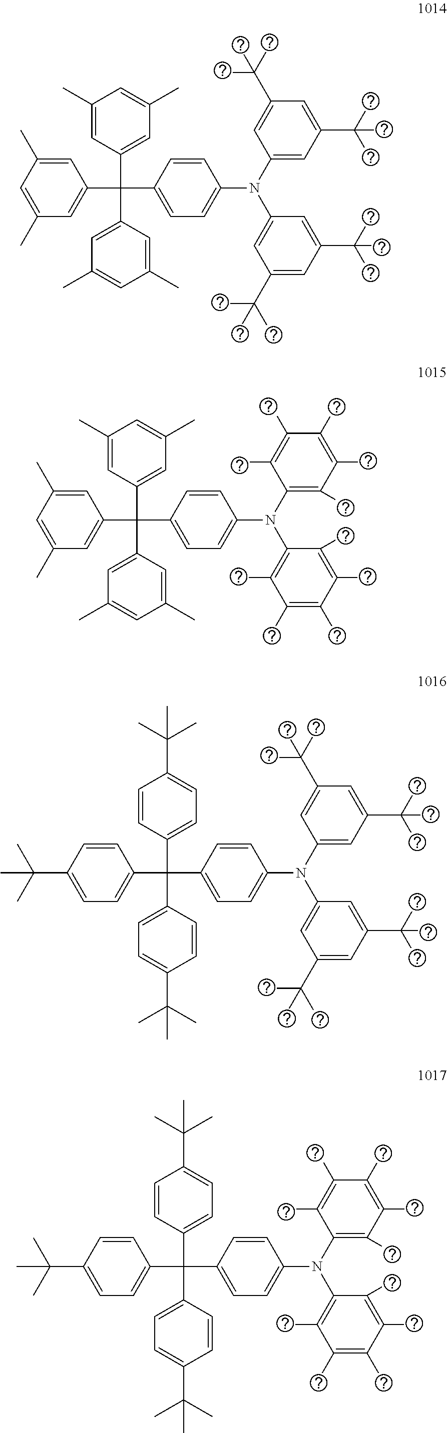

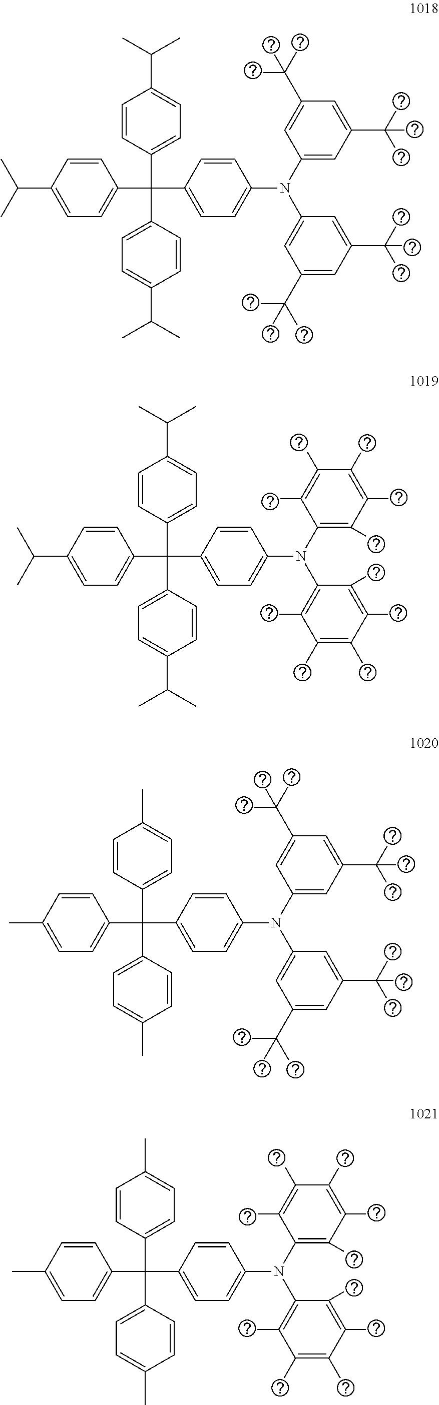

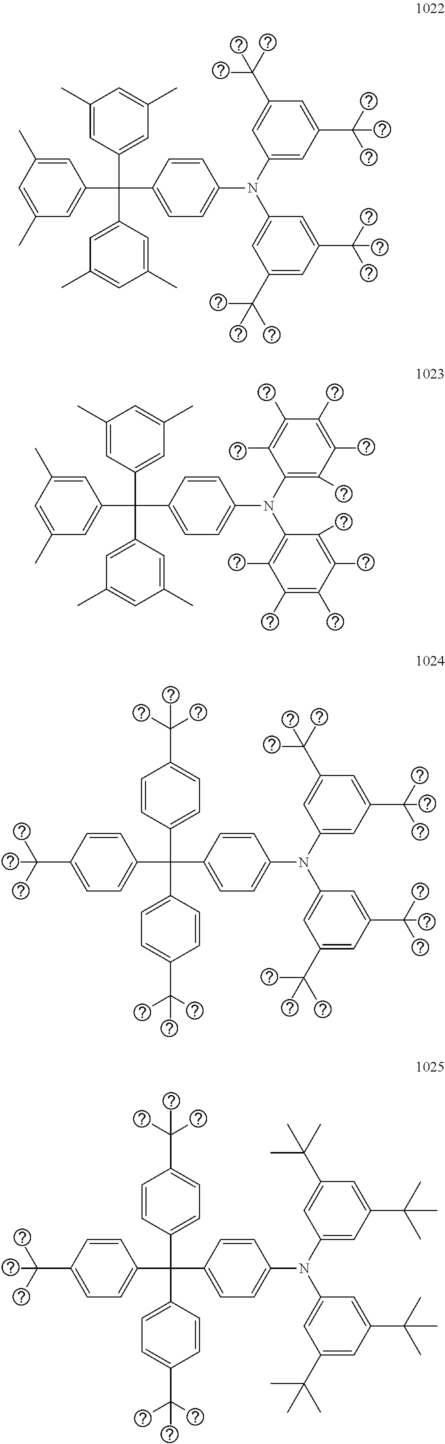

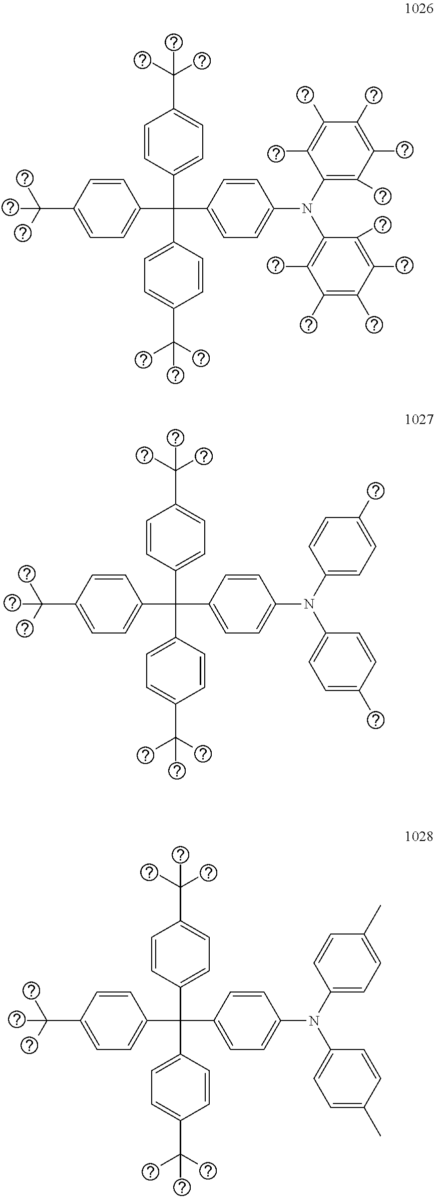

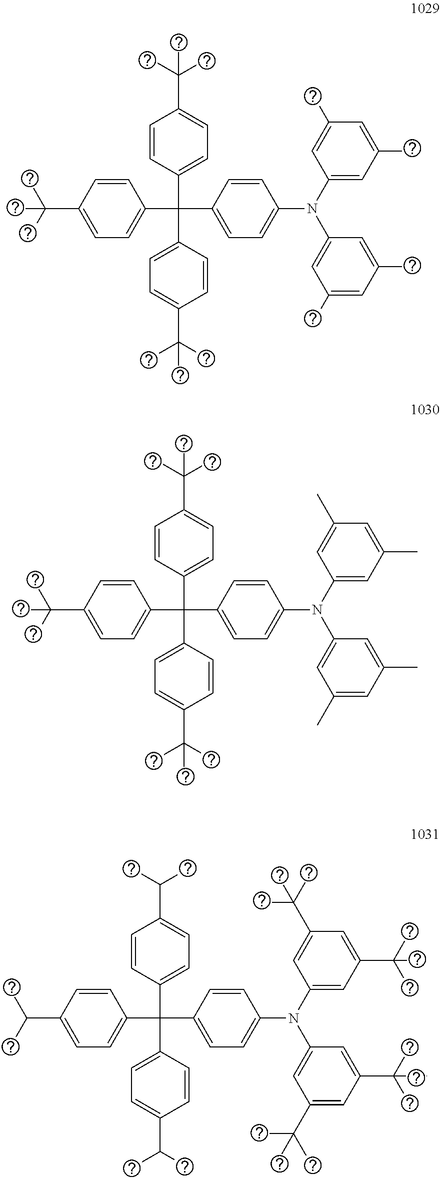

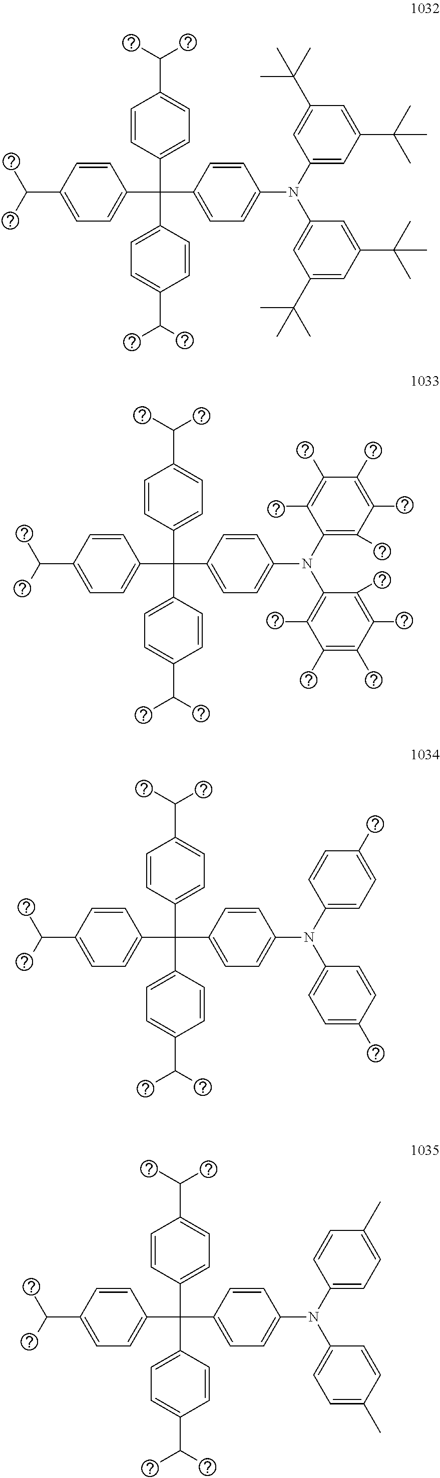

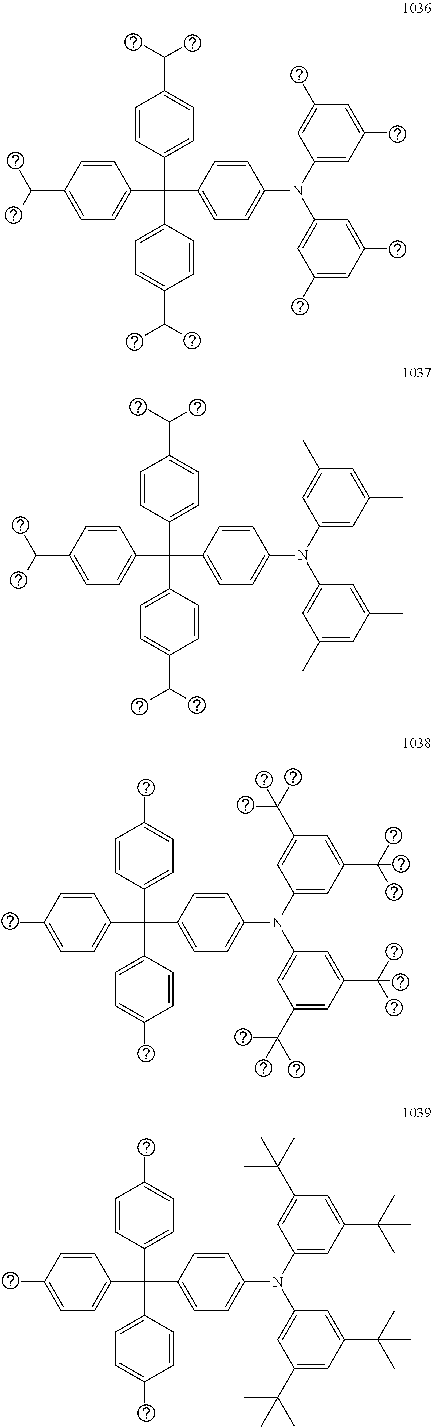

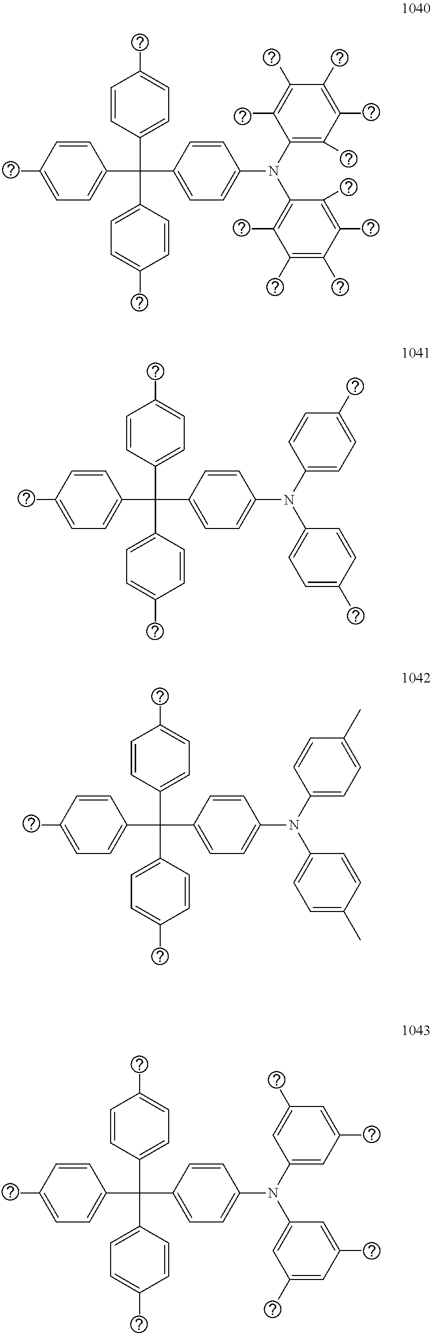

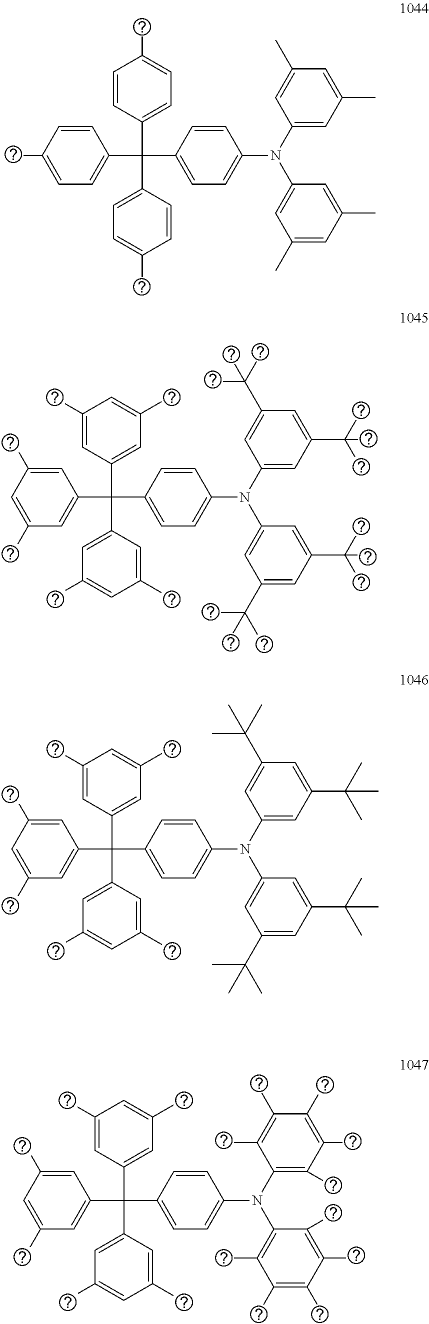

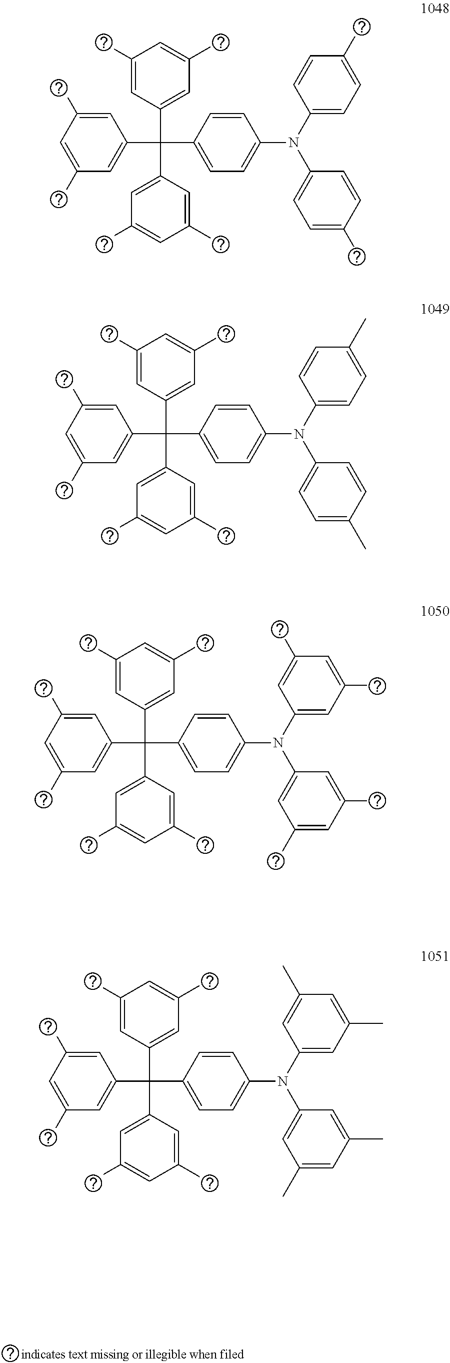 [SMILES: Cc1ccc(N(c2ccc(C)cc2)c2ccc(C(c3cc(C)cc(C)c3)(c3cc(C)cc(C)c3)c3cc(C)cc(C)c3)cc2)cc1 ; Cc1ccc(N(c2ccc(C)cc2)c2ccc(C(c3cc(C)cc(C)c3)(c3cc(C)cc(C)c3)c3cc(C)cc(C)c3)cc2)cc1 ; Cc1cc(C)cc(N(c2ccc(C(c3cc(C)cc(C)c3)(c3cc(C)cc(C)c3)c3cc(C)cc(C)c3)cc2)c2cc(C)cc(C)c2)c1 ; Cc1cc(C)cc(N(c2ccc(C(c3cc(C)cc(C)c3)(c3cc(C)cc(C)c3)c3cc(C)cc(C)c3)cc2)c2cc(C)cc(C)c2)c1]

Through the above synthetic example of the present invention, the results of the synthesis of additional organic compound (fluorine substitution) 1004 is shown in Table 17 below.

TABLE 17

| | | | | Obtained | |
|---|---|---|---|---|---|
| | | | | amount | |
| Compound | Reactant 1 | Reactant 2 | Product | (yield)) | [M + H]⁺ |
| 1004 | 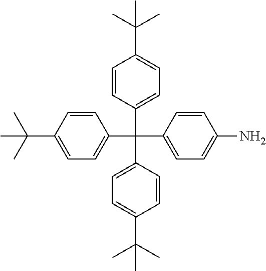 [SMILES: CC(C)(C)c1ccc(C(c2ccc(N)cc2)(c2ccc(C(C)(C)C)cc2)c2ccc(C(C)(C)C)cc2)cc1] | 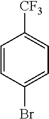 [SMILES: FC(F)(F)c1ccc(Br)cc1] | 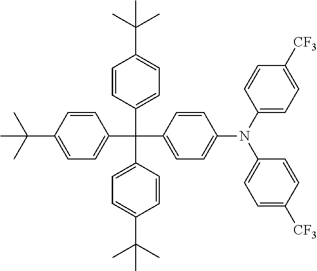 [SMILES: CC(C)(C)c1ccc(C(c2ccc(N(c3ccc(C(F)(F)F)cc3)c3ccc(C(F)(F)F)cc3)cc2)(c2ccc(C(C)(C)C)cc2)c2ccc(C(C)(C)C)cc2)cc1] | 13.2 g (80%) | 828.64 |

Through the above Experimental Example 1 of the present invention, the results of the refractive index and transmittance (%) of additional organic compound (fluorine substitution) 1004 is shown in Table 18 below.

TABLE 18

| Single film- | 400-410 nm | @460 nm | | @520 nm | | @620 nm | |
|---|---|---|---|---|---|---|---|
| forming | Transmittance | Refractive | Transmittance | Refractive | Transmittance | Refractive | Transmittance |
| material | (%) | index | (%) | index | (%) | index | (%) |
| Compound | 94 | 1.59 | 100 | 1.59 | 100 | 1.60 | 100 |
| 1004 | | | | | | | |

Referring to Table 8 above, examining the optical properties, it can be confirmed that additional organic compound (fluorine substitution) 1004 has low refractive indices of less than 1.6 and high transmittance of 100% at wavelengths of 460 nm, 520 nm and 620 nm.

In addition, compound (fluorine substitution) has transmittances of 90% or higher at wavelengths of 400 nm or more and 410 nm or less, minimizing the loss of light generated from the diode and realizing high efficiency.

Through these results, it can be confirmed that structure of chemical formula 1 of the present invention substituted fluorine, it has a low refractive index and a high transmittance of 100%. In addition, through this, high efficiency can be achieved in diode experiments.

What is claimed is:

1. A compound represented by the following Chemical Formula 1: 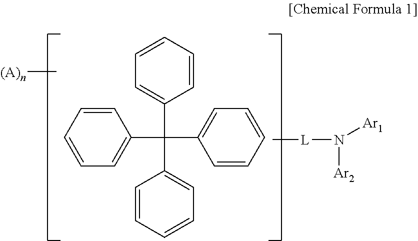
wherein in the Chemical Formula 1,
n is an integer of 1 to 20,
A is an alkyl group having 1 to 30 carbon atoms, or a cycloalkyl group having 3 to 20 carbon atoms,
L is selected from the group consisting of a single bond, a substituted or unsubstituted arylene group having 6 to 30 carbon atoms, a substituted or unsubstituted arylalkyl group having 7 to 30 carbon atoms, a substituted or unsubstituted heteroarylene group having 5 to 60 carbon atoms and a substituted or unsubstituted heteroarylalkylene group having 6 to 60 carbon atoms,
Ar₁ and Ar₂ are identical with or different from each other, and are each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms and a substituted or unsubstituted aryloxy group having 6 to 30 carbon atoms, or combined with an adjacent group from each other to form a substituted or unsubstituted ring, and
the substituents of A, Ar₁, and Ar₂ are each independently at least one selected from the group consisting of deuterium, a cyano group, a nitro group, a halogen group, a hydroxyl group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an arylalkyl group having 6 to 30 carbon atoms, a cycloalkyl group having 3 to 20 carbon atoms, a heterocycloalkyl group having 3 to 20 carbon atoms, an aryl group having 6 to 30 carbon atoms, a heteroaryl group having 2 to 30 carbon atoms, a heteroarylalkyl group having 3 to 30 carbon atoms, an alkoxy group having 1 to 30 carbon atoms, an alkylsilyl group having 1 to 30 carbon atoms, an arylsilyl group having 6 to 30 carbon atoms, and an aryloxy group having 6 to 30 carbon atoms, where when a plurality of substituents are introduced, the substituents are identical with or different from each other and combined with an adjacent group from each other to form a substituted or unsubstituted ring.

2. The compound of claim 1, wherein Chemical Formula 1 is selected from the group consisting of the compounds represented by the following Chemical Formulas 1-1 to 1-6:

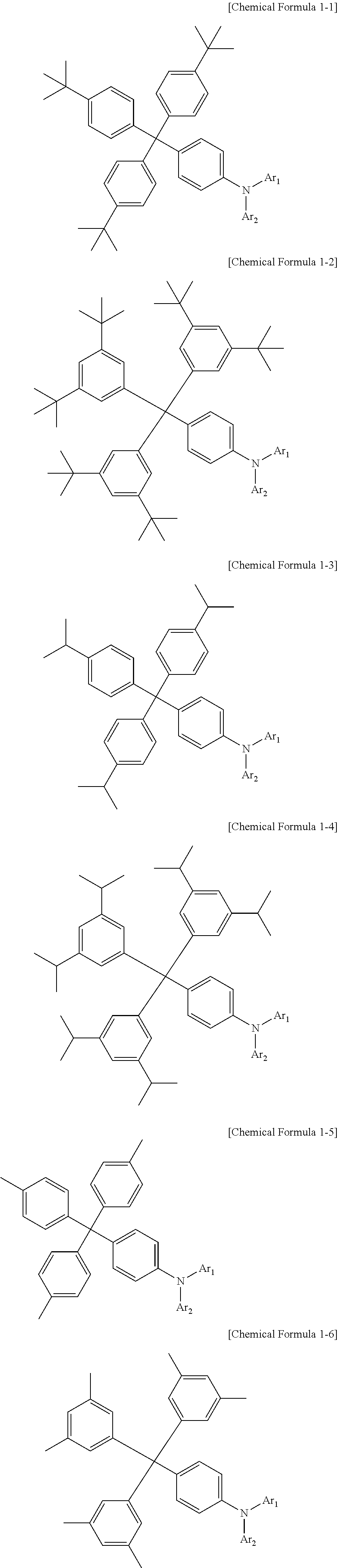

3. The compound of claim 1, wherein Ar₁ and Ar₂ are identical with or different from each other and are each independently selected from the group consisting of a substituted or unsubstituted alkyl group having 1 to 30 carbon atoms, a substituted or unsubstituted cycloalkyl group having 3 to 20 carbon atoms, a substituted or unsubstituted aryl group having 6 to 30 carbon atoms and a substituted or unsubstituted arylalkyl group having 7 to 30 carbon atoms.

4. The compound of claim 1, wherein at least one of Ar₁ and Ar₂ is selected from the group consisting of an unsubstituted alkyl group having 1 to 30 carbon atoms, a substituted or unsubstituted cycloalkyl group having 3 to 20 carbon atoms, and a substituted or unsubstituted arylalkyl group having 7 to 30 carbon atoms.

5. The compound of claim 1, wherein Ar₁ and Ar₂ do not comprise a fused aryl structure.

6. The compound of claim 1, wherein Ar₁ and Ar₂ are each independently selected from the group consisting of the substituents represented by the following F1 to F57: 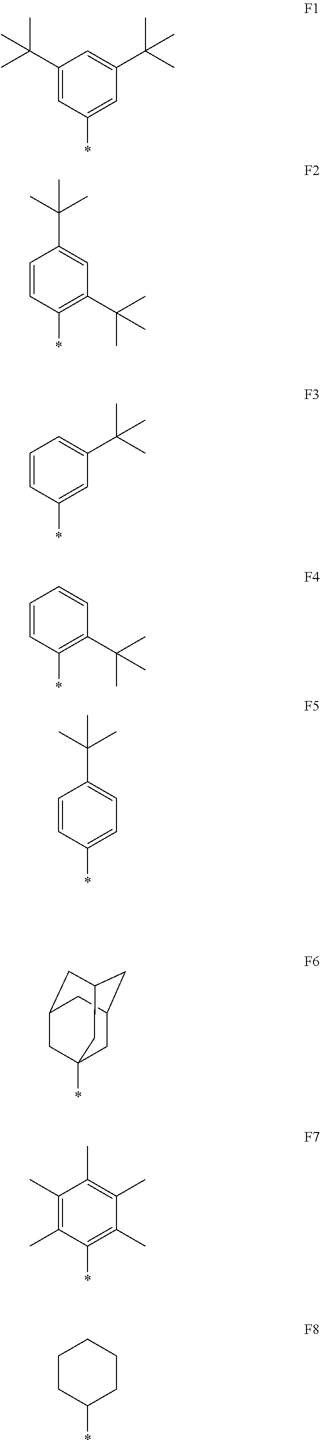 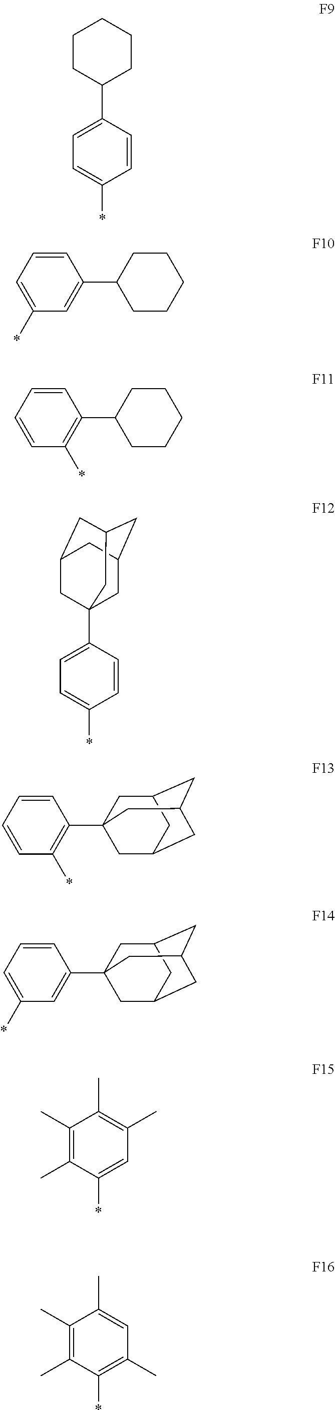 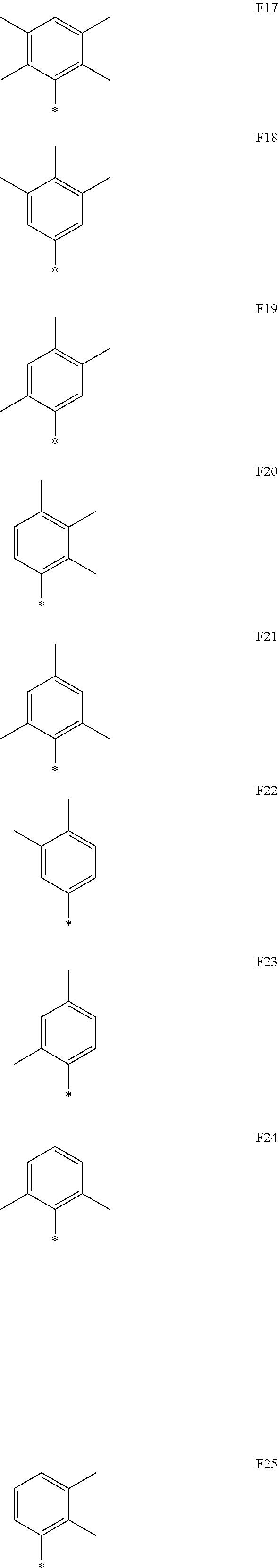 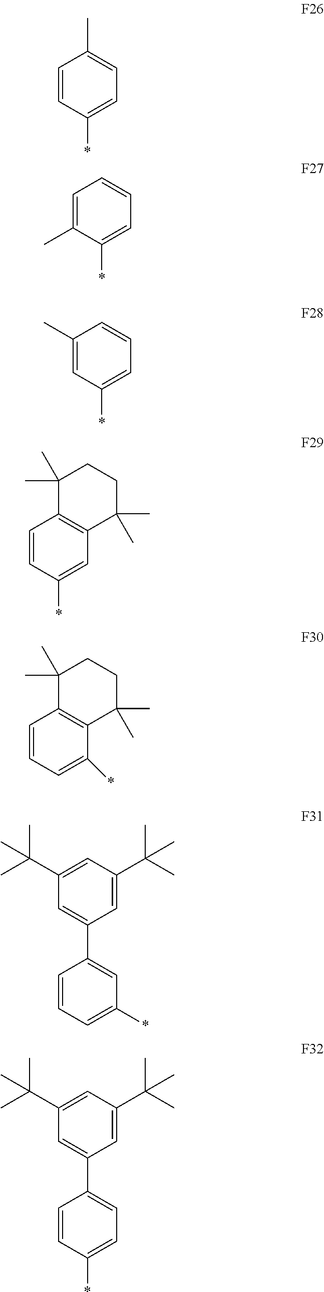 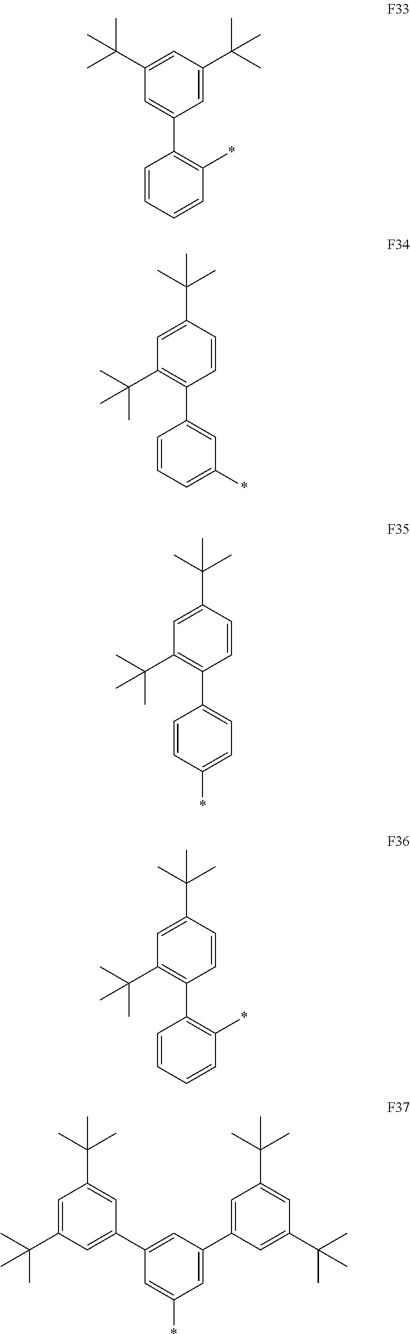 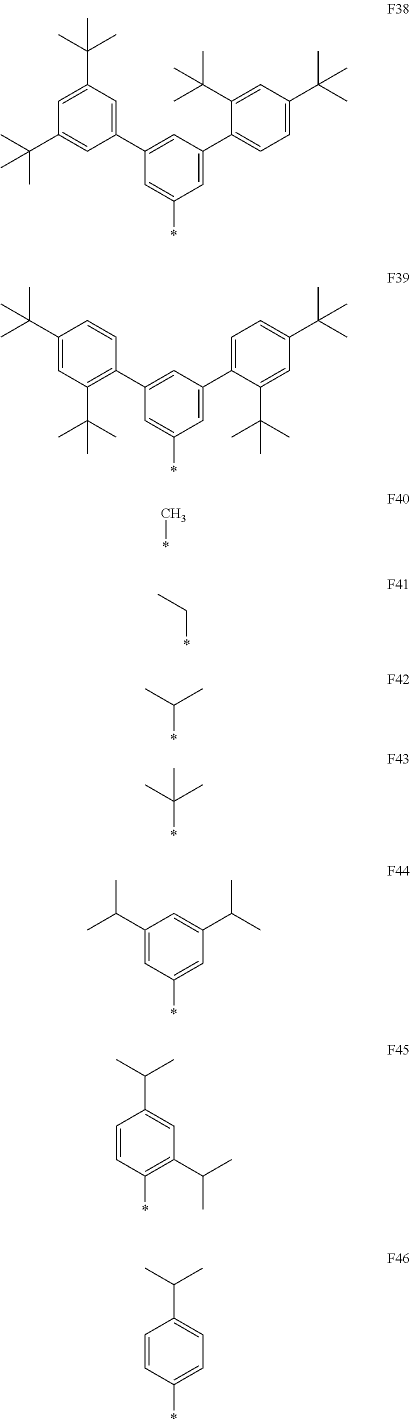 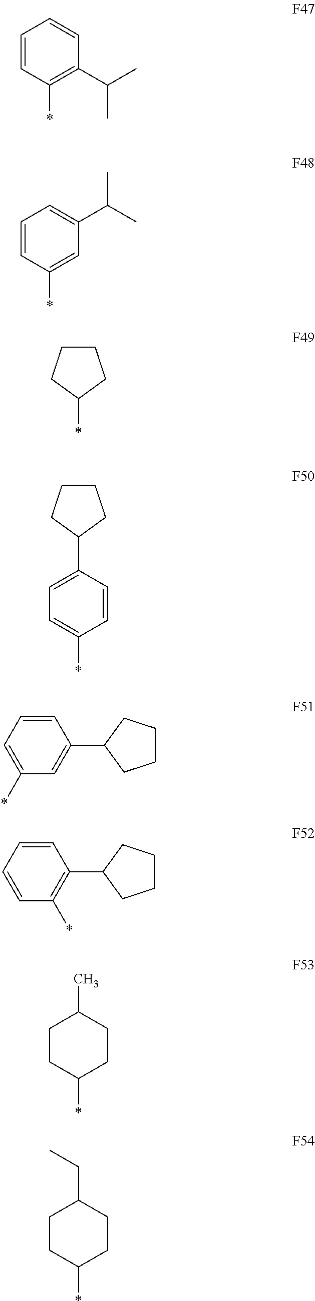 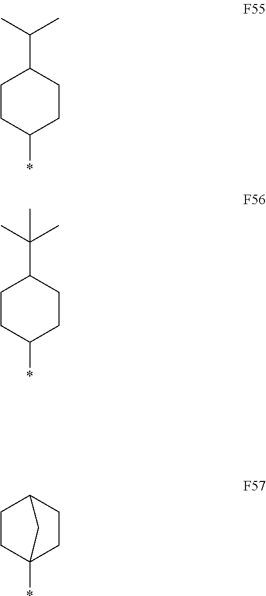
where
* indicates a part being combined.

7. An organic light-emitting diode, comprising:
a first electrode;
a second electrode facing the first electrode;
at least one organic material layer positioned on the inner side of the first electrode and the second electrode; and
a capping layer positioned on the outer side of at least one of the first electrode and the second electrode, wherein
the capping layer comprises the compound of claim 1.

8. The organic light-emitting diode of claim 7, wherein the organic material layer comprises at least one layer of a hole injection layer, a hole transport layer, an electron blocking layer, a light-emitting layer, a hole blocking layer, an electron transport layer, or an electron injection layer.

9. A display device comprising the organic light-emitting diode of claim 7.
